(19) United States
(12) Patent Application Publication
(10) Pub. No.: US 2005/0119210 A1
(43) Pub. Date: Jun. 2, 2005

(54) Compositions and methods for diagnosing and treating cancers

(76) Inventors: Xiaobing Be, Medford, MA (US); Wei Liu, Sudbury, MA (US); Donna K. Slonim, North Andover, MA (US); Steve Howes, Cambridge, MA (US)

Correspondence Address: NIXON PEABODY, LLP, 401 9TH STREET, NW, SUITE 900, WASHINGTON, DC, 20004-2128, US

(21) Appl. No.: 10/847,918

(22) Filed: May 19, 2004

Related U.S. Application Data
(60) Provisional application No. 60/471,729, filed on May 20, 2003.

(51) Int. Cl. A61K 48/00; C12Q 1/68; A61K 39/395; A61K 38/17; G01N 33/574

(52) U.S. Cl. 514/44; 514/12; 424/155.1; 435/6; 435/7.23

(57) ABSTRACT

Compositions and methods for diagnosing, monitoring, or treating cancers. Genes encoding transmembrane proteins that are over-expressed in colon, lung, breast, prostate, liver, stomach, esophagus, or kidney cancer tissues are identified. These transmembrane genes can be used as biological markers for the detection or diagnosis of cancers. These genes can also serve as targets for cancer therapeutics. In addition, these genes can also be used for screening for anti-cancer drugs.

### CROSS REFERENCE TO RELATED APPLICATIONS

This application claims priority from and incorporates by reference the entire disclosures of U.S. Provisional Application Ser. No. 60/471,729, filed May 20, 2003.

All materials recorded in compact discs labeled “Copy 1” and “Copy 2” are incorporated herein by reference in their entireties. Each of the compact discs includes a file entitled “Sequence Listing.ST25.txt” (2,406 KB, created on May 18, 2004).

### TECHNICAL FIELD

The present invention relates generally to the diagnosis and treatment of cancers. In particular, the present invention relates to genes encoding cancer-related transmembrane proteins that are overexpressed in cancer tissues and methods of using these genes for detecting, monitoring and treating cancers.

### BACKGROUND OF THE INVENTION

Cancer is a significant health problem throughout the world. The most frequently diagnosed cancers include colon cancer, lung cancer, breast cancer, prostate cancer, liver cancer, stomach cancer, esophagus cancer, and kidney cancer. These cancers represent a majority of cancers diagnosed in the U.S. population and account for over 90% of the cancer-related death in the U.S.

The five-year survival rate for patients with colon cancer detected in an early localized stage is 92%; unfortunately, only 37% of colon cancer is diagnosed at this stage. The survival rate drops to 64% if the cancer is allowed to spread to adjacent organs or lymph nodes, and to 7% in patients with distant metastases. Therefore, there is a need for early detection and diagnosis of cancers. Current therapies, such as chemotherapy, surgery or radiation, are inadequate for many cancer patients. Accordingly, there is another need for improved methods to treat cancers.

### SUMMARY OF THE INVENTION

The present invention provides compositions and methods for diagnosing and treating cancers. Cancers amenable to the present invention include colon, lung, breast, prostate, liver, stomach, esophagus, and kidney cancers. The present invention is based on the identification of transmembrane proteins that are over-expressed in at least one cancer tissue. These proteins are hereinafter referred to as “cancer-related transmembrane proteins” or “CRTPs.” The genes that encode these proteins are referred to as “cancer-related transmembrane protein genes” or “CRTPGs,” and the polynucleotides transcribed from or encoded by the CRTPGs are referred to as “cancer-related transmembrane protein nucleotides” or “CRTPNs.”

In one aspect, the present invention provides pharmaceutical compositions that are useful for treating or preventing cancers. In many embodiments, the pharmaceutical compositions of the present invention include a pharmaceutically acceptable carrier and an active component selected from the group consisting of (1) an agent capable of modulating the expression or protein activity of a CRTPG; (2) an antibody specific for a polypeptide encoded by a CRTPG; and (3) a T cell activated by a polypeptide encoded by a CRTPG. Example CRTPGs that are suitable for the present invention are depicted in Table 1a. In one embodiment, the CRTPG(s) is selected from the group consisting of ABCC4, C20orf103, CACNA1D, CDH6, CST, ENPP3, FLJ11856, GPR54, HAVCR1, SLC6A3, SLC30A4, TRG@, and TRPM4.

In another embodiment, the pharmaceutical compositions of the present invention include a polynucleotide capable of inhibiting or decreasing the expression of a CRTPG by RNA interference or an antisense mechanism. Example RNAi sequences include, but are not limited to, the siRNAs depicted in Table 3. In yet another embodiment, the pharmaceutical compositions of the present invention include an antibody which is conjugated with a toxic moiety. The antibody targets and kills cancer cells that overexpress a protein recognizable by the antibody. In still another embodiment, the pharmaceutical compositions of the present invention include a small molecule or drug that inhibits or reduces a protein activity of a CRTPG. In many examples, the inhibition is mediated by direct or indirect binding of the small molecule or drug to a protein encoded by the CRTPG.

In many other embodiments, the pharmaceutical compositions of the present invention are vaccine formulations. In one embodiment, the vaccines include a polypeptide containing an immunogenic fragment encoded by a CRTPG, or an expression vector encoding the polypeptide.

The present invention also features methods for treating or preventing cancers. These methods include the administration of a therapeutically or prophylactically effective amount of a pharmaceutical composition of the present invention to a patient in need thereof. In many embodiments, the patient has at least one cancer selected from colon cancer, lung cancer, breast cancer, prostate cancer, liver cancer, kidney cancer, stomach cancer, and esophageal cancer, and the pharmaceutical composition is prepared based on the CRTPG(s) selected according to Table 1b. In one embodiment, a pharmaceutical composition of the present invention is administered to the patient to inhibit or reduce the expression or protein activity of a CRTPG in cancer cells. In another embodiment, a pharmaceutical composition of the present invention is administered to the patient to elicit an immune response against cancer cells that overexpress a CRTPG.

In another aspect, the present invention provides methods that are useful for detecting, evaluating, or screening for compounds or other molecules that modulate the expression or protein activity of a CRTPG. The methods include contacting an agent of interest with cells expressing the CRTPG, and comparing the expression or protein activity of the CRTPG before and after the contact to determine if the agent is a modulator of the CRTPG. In one embodiment, the cells employed in the present invention are infected by at least one cancer selected from colon cancer, lung cancer, breast cancer, prostate cancer, liver cancer, kidney cancer, stomach cancer, and esophageal cancer, and the CRTPG is overexpressed in the cancer cells as compared to the cancer-free counterparts.

In still another aspect, the present invention provides methods for detecting or diagnosing cancers or monitoring the efficacy or effectiveness of a cancer treatment. These methods include detecting an expression profile of at least one CRTPG in a biological sample of a subject of interest, and comparing the expression profile to a reference expression profile of the CRTPG. In one embodiment, the subject of interest is a cancer patient, and the biological sample is a cancer tissue sample. In another embodiment, the cancer patient is treated by a therapy, and the effectiveness of the therapy is evaluated by monitoring the changes in the expression of CRTPGs that are overexpressed in untreated cancer cells. In still another embodiment, the reference expression profile is an average expression profile of the CRTPG in biological samples of cancer-free humans. In many embodiments, the expression profiles being compared are prepared from the same type of tissue by using the same or comparable methodology. Methods suitable for detecting expression profiles include, but are not limited to, RT-PCR, nucleic acid arrays, and immunoassays.

In addition, the present invention features cancer diagnostic kits or devices. In many embodiments, a diagnostic kit or device of the present invention includes a polynucleotide probe capable of hybridizing under stringent conditions to a gene selected from the group consisting of ABCC4, C20orf103, CACNA1D, CDH6, CST, ENPP3, FLJ11856, GPR54, HAVCR1, SLC6A3, SLC03A4, TRG@, and TRPM4, or an antibody specific for a polypeptide encoded by the gene. The polypeptide probes or antibodies in a diagnostic kit of the present invention can be stably attached to one or more substrate supports to make a nucleic acid or protein array.

Other features, objects, and advantages of the present invention are apparent in the detailed description that follows. It should be understood, however, that the detailed description, while indicating preferred embodiments of the present invention, is given by way of illustration only, not limitation. Various changes and modifications within the scope of the invention will become apparent to those skilled in the art from the detailed description.

### BRIEF DESCRIPTION OF THE DRAWINGS

The patent or application file contains at least one drawing executed in color. Copies of this patent or patent application publication with color drawings will be provided by the U.S. Patent and Trademark Office upon request and payment of the necessary fee. The drawings are provided for illustration, not limitation.

FIG. 1A shows the e-northern result of ABCC4 expression in cancerous versus cancer-free tissues. Like all other e-northern results, the ABCC4 e-northern result is composed of three parts, left, middle, and right. The left part lists the sample sets used to obtain the plot. It includes the sample set name and number of samples (in parenthesis) in the sample set. The middle part uses bars to describe the “present” percentage of individual samples in the sample set. The present percentage is proportional to the filled length of the bar, which is also labeled by the number below the bar. The right part is composed of numerous plots, each of which corresponds to one respective sample set. The vertical bar is positioned along the x-axis according to the expression value of each individual sample. The vertical bars are color coded as follows: red means absent, blue means present, and yellow means marginal. There are five numbers below the color-coded bars. The middle one is the median of expression values, the number to the left and next to the median is the 25% quantile, and the number to the right and next to the median is the 75% quantile. The leftmost number represents the position that is 1.5-fold interquantile distance away from the 25% quantile. The rightmost number represents the position that is 1.5-fold interquantile distance away from the 75% quantile.

FIG. 1B is the global analysis plot of ABCC4 expression in cancerous tissues. Like other global analysis plots, the x-axis in the ABCC4 plot describes the sample number, and the y-axis describes the expression value. Each light black dot represents the expression value of a single sample. The mean expression value for an individual tissue type is represented by a horizontal colored bar. The position of each colored bar indicates the mean expression value. The existence of blue dots below the mean bars means that the expression of those samples is called “present,” as described in Example 1.

FIG. 1C depicts the global analysis plot of ABCC4 expression in cancer-free tissues.

FIG. 1D shows the TMHMM profile of a human ABCC4 polypeptide consisting of the amino acid sequence recited in SEC ID NO: 16. A typical TMHMM plot contains two parts. The first part of the TMHMM profile gives statistics and locations of the predicted transmembrane helices and intervening loop regions. The second part of the TMHMM profile is a plot of probabilities, which shows the posterior probabilities of inside the membrane, outside the membrane, and transmembrane helix. At the top of the plot (between 1 and 1.2) is the N-best prediction.

FIG. 2A demonstrates the e-northern result of C20orf103 expression in cancerous versus cancer-free tissues.

FIG. 2B illustrates the global analysis of C20orf103 expression in cancerous tissues.

FIG. 2C shows the global analysis of C20orf103 expression in cancer-free tissues.

FIG. 2D indicates the TMHMM profile of a human C20orf103 polypeptide consisting of the amino acid sequence recited in SEC ID NO:17.

FIG. 3A demonstrates the e-northern result of CACNA1D expression in cancerous versus cancer-free tissues.

FIG. 3B illustrates the global analysis of CACNA1D expression in cancerous tissues.

FIG. 3C shows the global analysis of CACNA1D expression in cancer-free tissues.

FIG. 3D is the TMHMM profile of a human CACNA1D polypeptide consisting of the amino acid sequence recited in SEC ID NO:18.

FIG. 4A demonstrates the e-northern result of CDH6 expression in cancerous versus cancer-free tissues.

FIG. 4B illustrates the global analysis of CDH6 expression in cancerous tissues.

FIG. 4C shows the global analysis of CDH6 expression in cancer-free tissues.

FIG. 4D indicates the TMHMM profile of a human CDH6 polypeptide consisting of the amino acid sequence recited in SEC ID NO:19.

FIG. 5A depicts the e-northern result of CST expression in cancerous versus cancer-free tissues.

FIG. 5B illustrates the global analysis of CST expression in cancerous tissues.

FIG. 5C shows the global analysis of CST expression in cancer-free tissues.

FIG. 5D indicates the TMHMM profile of a human CST polypeptide consisting of the amino acid sequence recited in SEC ID NO:20.

FIG. 6A demonstrates the e-northern result of ENPP3 expression in cancerous versus cancer-free tissues.

FIG. 6B illustrates the global analysis of ENPP3 expression in cancerous tissues.

FIG. 6C shows the global analysis of ENPP3 expression in cancer-free tissues.

FIG. 6D indicates the TMHMM profile of a human ENPP3 polypeptide consisting of the amino acid sequence recited in SEC ID NO:21.

FIG. 7A demonstrates the e-northern result of FLJ11856 expression in cancerous versus cancer-free tissues.

FIG. 7B illustrates the global analysis of FLJ11856 expression in cancerous tissues.

FIG. 7C shows the global analysis of FLJ11856 expression in cancer-free tissues.

FIG. 7D depicts the TMHMM profile of a human FLJ11856 polypeptide consisting of the amino acid sequence recited in SEC ID NO:22.

FIG. 8A demonstrates the e-northern result of FOLH1 expression in cancerous versus cancer-free tissues.

FIG. 8B illustrates the global analysis of FOLH1 expression in cancerous tissues.

FIG. 8C shows the global analysis of FOLH1 expression in cancer-free tissues.

FIG. 8D indicates the TMHMM profile of a human FOLH1 polypeptide consisting of the amino acid sequence recited in SEC ID NO:23.

FIG. 9A demonstrates the e-northern result of GPR54 expression in cancerous versus cancer-free tissues.

FIG. 9B illustrates the global analysis of GPR54 expression in cancerous tissues.

FIG. 9C shows the global analysis of GPR54 expression in cancer-free tissues.

FIG. 9D indicates the TMHMM profile of a human GPR54 polypeptide consisting of the amino acid sequence recited in SEC ID NO:24.

FIG. 10A demonstrates the e-northern result of HAVCR1 expression in cancerous versus cancer-free tissues.

FIG. 10B illustrates the global analysis of HAVCR1 expression in cancerous tissues.

FIG. 10C shows the global analysis of HAVCR1 expression in cancer-free tissues.

FIG. 10D is the TMHMM profile of a human HAVCR1 polypeptide consisting of the amino acid sequence recited in SEC ID NO:25.

FIG. 11A demonstrates the e-northern result of OR51E2 expression in cancerous versus cancer-free tissues.

FIG. 11B illustrates the global analysis of OR51E2 expression in cancerous tissues.

FIG. 11C shows the global analysis of OR51E2 expression in cancer-free tissues.

FIG. 11D indicates the TMHMM profile of a human OR51E2 polypeptide consisting of the amino acid sequence recited in SEC ID NO:26.

FIG. 12A demonstrates the e-northern result of SLC6A3 expression in cancerous versus cancer-free tissues.

FIG. 12B illustrates the global analysis of SLC6A3 expression in cancerous tissues.

FIG. 12C shows the global analysis of SLC6A3 expression in cancer-free tissues.

FIG. 12D depicts the TMHMM profile of a human SLC6A3 polypeptide consisting of the amino acid sequence recited in SEC ID NO:27.

FIG. 13A demonstrates the e-northern result of SLC30A4 expression in cancerous versus cancer-free tissues.

FIG. 13B illustrates the global analysis of SLC30A4 expression in cancerous tissues.

FIG. 13C shows the global analysis of SLC30A4 expression in cancer-free tissues.

FIG. 13D indicates the TMHMM profile of a human SLC30A4 polypeptide consisting of the amino acid sequence recited in SEC ID NO:28.

FIG. 14A demonstrates the e-northern result of TRG@ expression in cancerous versus cancer-free tissues.

FIG. 14B illustrates the global analysis of TRG@ expression in cancerous tissues.

FIG. 14C shows the global analysis of TRG@ expression in cancer-free tissues.

FIG. 14D indicates the TMHMM profile of a human TRG@ polypeptide consisting of the amino acid sequence recited in SEC ID NO:29.

FIG. 15A demonstrates the e-northern result of TRPM4 expression in cancerous versus cancer-free tissues.

FIG. 15B illustrates the global analysis of TRPM4 expression in cancerous tissues.

FIG. 15C shows the global analysis of TRPM4 expression in cancer-free tissues.

FIG. 15D is the TMHMM profile of a human TRPM4 polypeptide consisting of the amino acid sequence recited in SEC ID NO:30.

### DETAILED DESCRIPTION OF THE INVENTION

The present invention provides compositions and methods for the diagnosis, treatment, and prevention of cancers. The present invention also provides methods for the identification or evaluation of anti-cancer drugs. Various aspects of the present invention are described in further detail in the following subsections. The use of subsections is not meant to limit the invention; subsections may apply to any aspect of the invention.

Cancer-Related Transmembrane Proteins (CRTPs) and Cancer-Related Transmembrane Protein Genes (CRTPGs)

Gene expression data in eight malignant tumor sample sets (i.e., breast, colon, esophagus, kidney, liver, lung, prostate, and stomach cancer sample sets) was compared to the corresponding normal counterparts in the Gene Logic BioExpress™ database. Fifteen CRTPGs were identified as showing higher average expression levels in specific tumor specimen(s) relative to the corresponding cancer-free tissue sample(s). The expression of each of these genes is increased by more than two-fold in at least one malignant tumor sample set as compared to the cancer-free counterpart, and the average expression level of the gene in the malignant sample set is greater than that in any of the eight cancer-free sample sets. The CRTPGs thus identified are depicted in Table 1a. Exemplary cDNA and amino acid sequences encoded by these genes are also provided.

TABLE 1a
Cancer-related transmembrane protein genes (CRTPGs)

| Gene Symbol | Locuslink. | cDNA Sequence | Amino Acid Sequence |
|---|---|---|---|
| ABCC4 | 10257 | SEQ ID NO: 1 | SEQ ID NO: 16 |
| C20orf103 | 24141 | SEQ ID NO: 2 | SEQ ID NO: 17 |
| CACNA1D | 776 | SEQ ID NO: 3 | SEQ ID NO: 18 |
| CDH6 | 1004 | SEQ ID NO: 4 | SEQ ID NO: 19 |
| CST | 9514 | SEQ ID NO: 5 | SEQ ID NO: 20 |
| ENPP3 | 5169 | SEQ ID NO: 6 | SEQ ID NO: 21 |
| FLJ11856 | 79581 | SEQ ID NO: 7 | SEQ ID NO: 22 |
| FOLH1 | 2346 | SEQ ID NO: 8 | SEQ ID NO: 23 |
| GPR54 | 84634 | SEQ ID NO: 9 | SEQ ID NO: 24 |
| HAVCR1 | 26762 | SEQ ID NO: 10 | SEQ ID NO: 25 |
| OR51E2 | 81285 | SEQ ID NO: 11 | SEQ ID NO: 26 |
| SLC6A3 | 6531 | SEQ ID NO: 12 | SEQ ID NO: 27 |
| SLC30A4 | 7782 | SEQ ID NO: 13 | SEQ ID NO: 28 |
| TRG@ | 6965 | SEQ ID NO: 14 | SEQ ID NO: 29 |
| TRPM4 | 54795 | SEQ ID NO: 15 | SEQ ID NO: 30 |

ABCC4 (ATP-binding cassette, sub-family C (CFTR/MRP), member 4) encodes a protein which is a member of the superfamily of ATP-binding cassette (ABC) transporters. ABC proteins transport various molecules across extra- and intra-cellular membranes. ABC genes are divided into seven distinct subfamilies (ABC1, MDR/TAP, MRP, ALD, OABP, GCN20, White). ABCC4 is a member of the MRP subfamily which is involved in multi-drug resistance. This protein is an apical organic anion transporter and may play a role in cellular detoxification as a pump for its substrate, organic anions. It is also a putative efflux pump for cAMP and cGMP in human kidney proximal tubules, and a common efflux system for both methotrexate and certain nucleotide analogues. It was reported that ABCC4 can mediate the export of glutathione (GSH). Depletion of intracellular GSH adversely affects the export of cAMP by ABCC4. Resistance to nucleoside analogues is also adversely affected by depletion of cellular GSH. It has been suggested that ABCC4 may play a physiological role in the transport of dehydroepiandrosterone-3-sulfate, conjugated steroids, and bile acids.

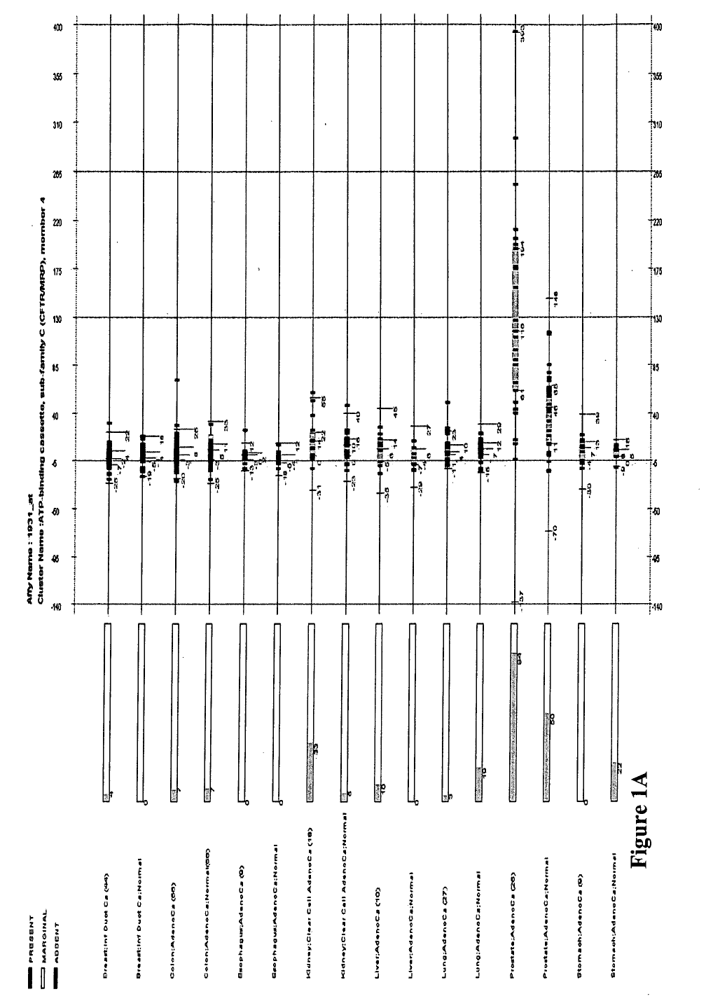

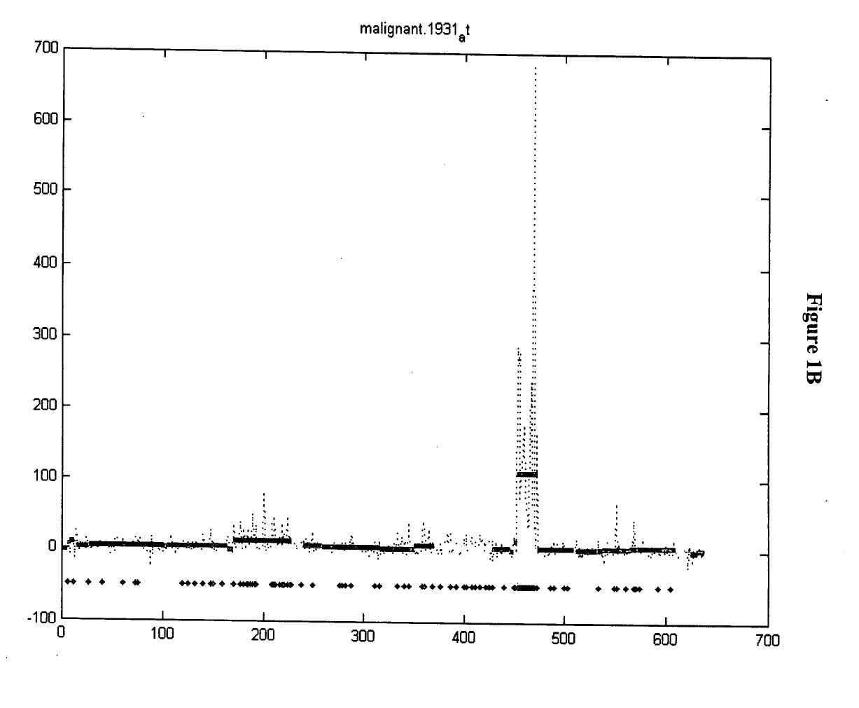

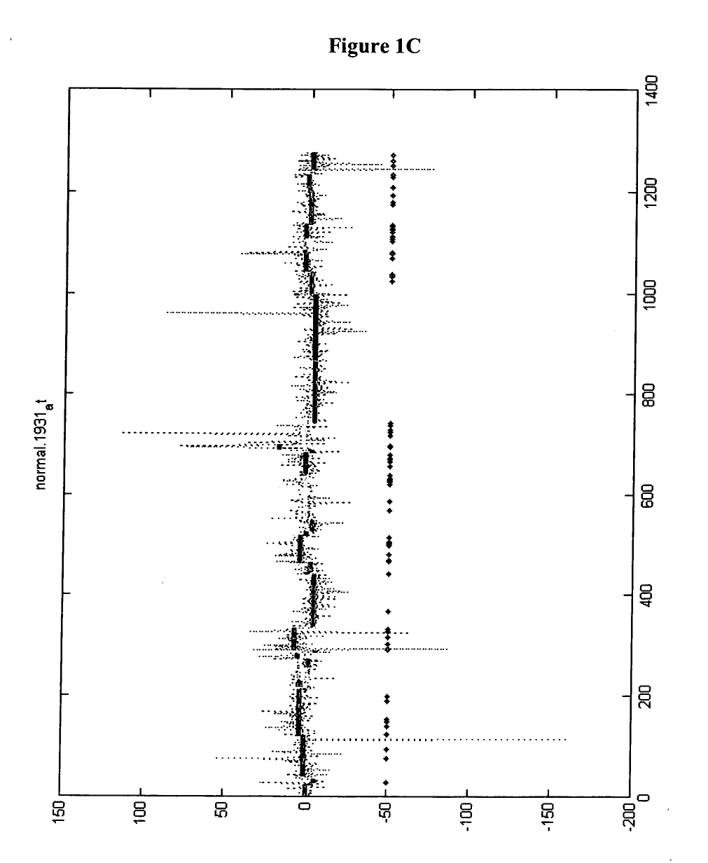

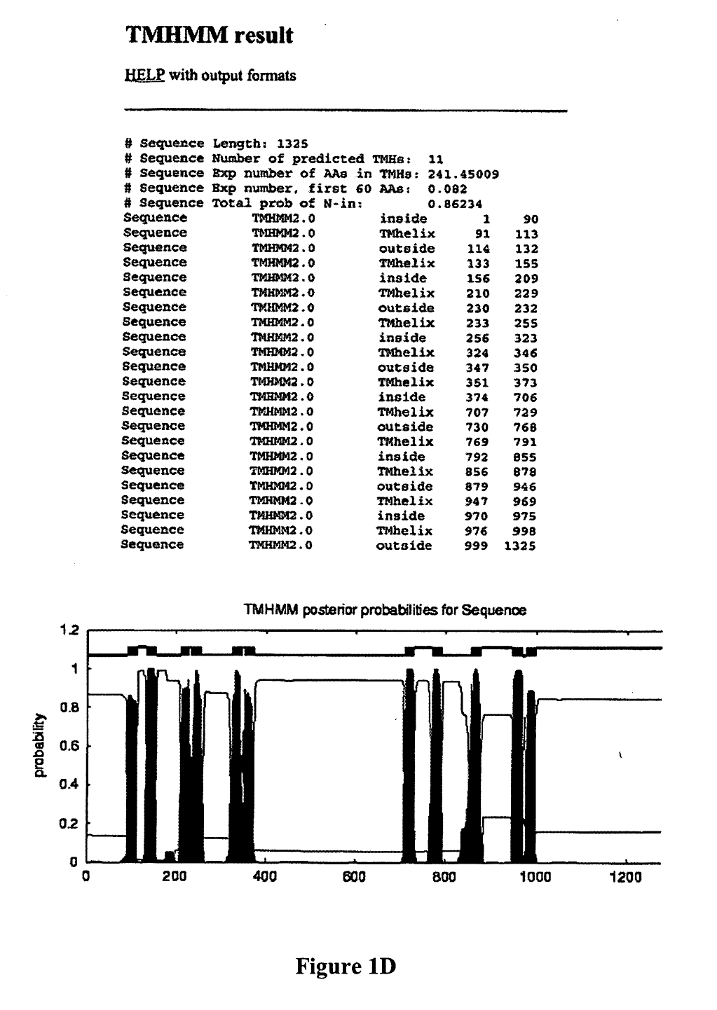

The expression levels of ABCC4 in various tumor and control tissue samples are depicted in FIGS. 1A-1C. The TMHMM profile of an ABCC4 protein is shown in FIG. 1D. As shown in FIG. 1A, the median expression level of ABCC4 in prostate cancer samples is at least 2.5 fold higher than that in the corresponding normal prostate samples. Hence, ABCC4 can be used as a diagnostic marker and drug target for prostate cancer.

C20orf103 (chromosome 20 open reading frame 103) is similar to S68401 (cattle) glucose induced gene. The protein encoded by the C20orf103 gene shares low similarity to a region of murine lysosome-associated membrane glycoprotein (Lamp1). The specific function of this protein has not yet been determined.

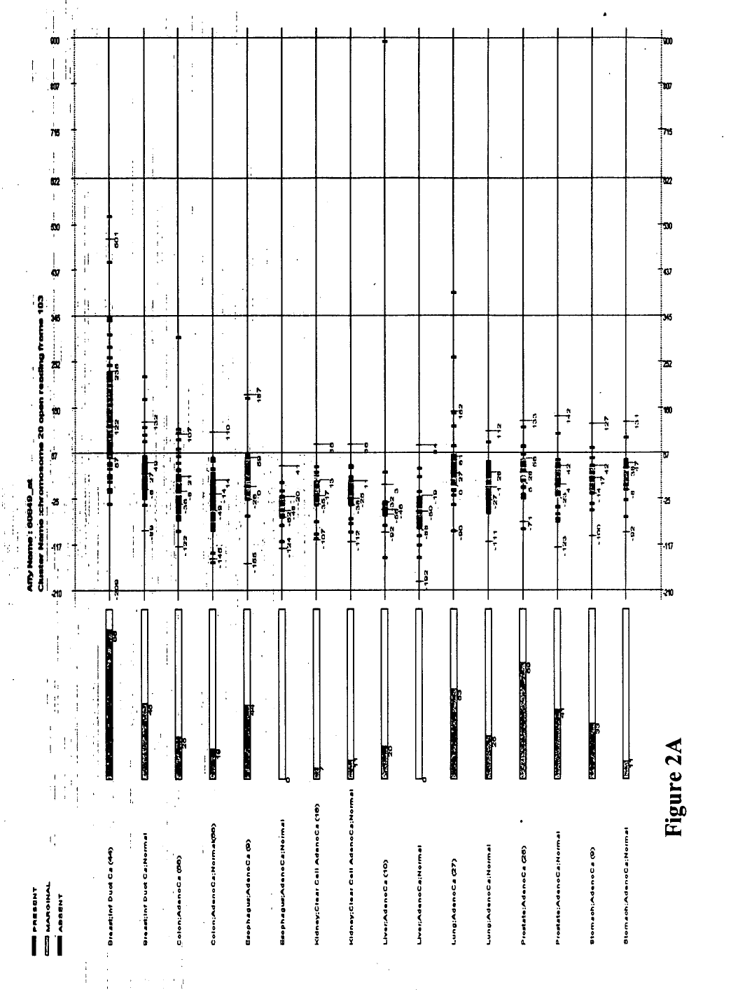

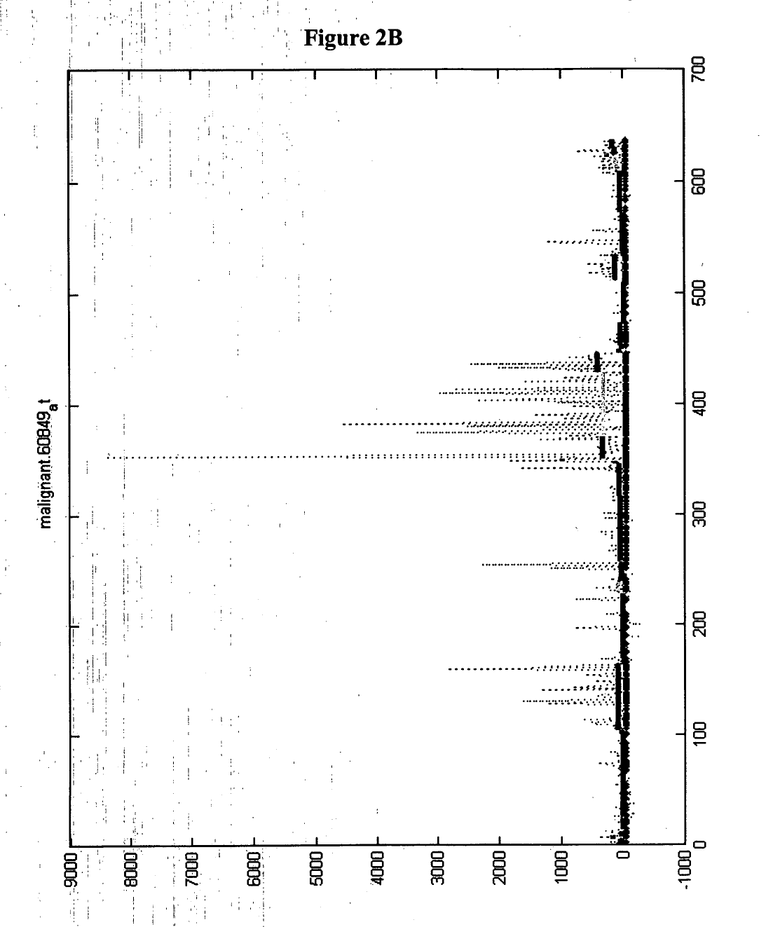

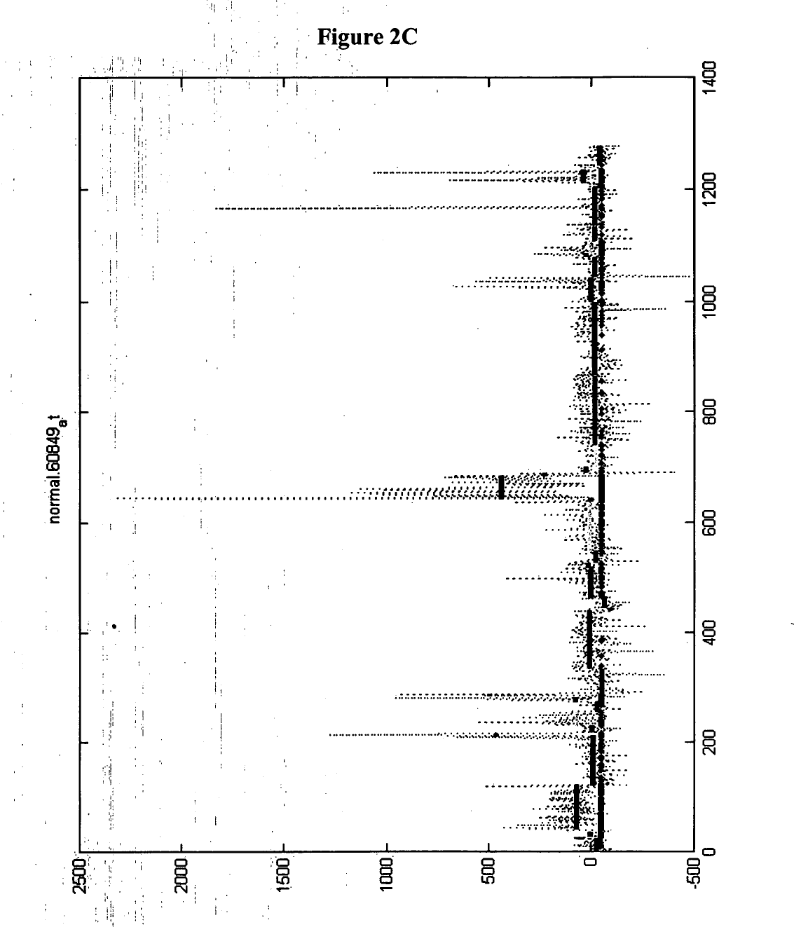

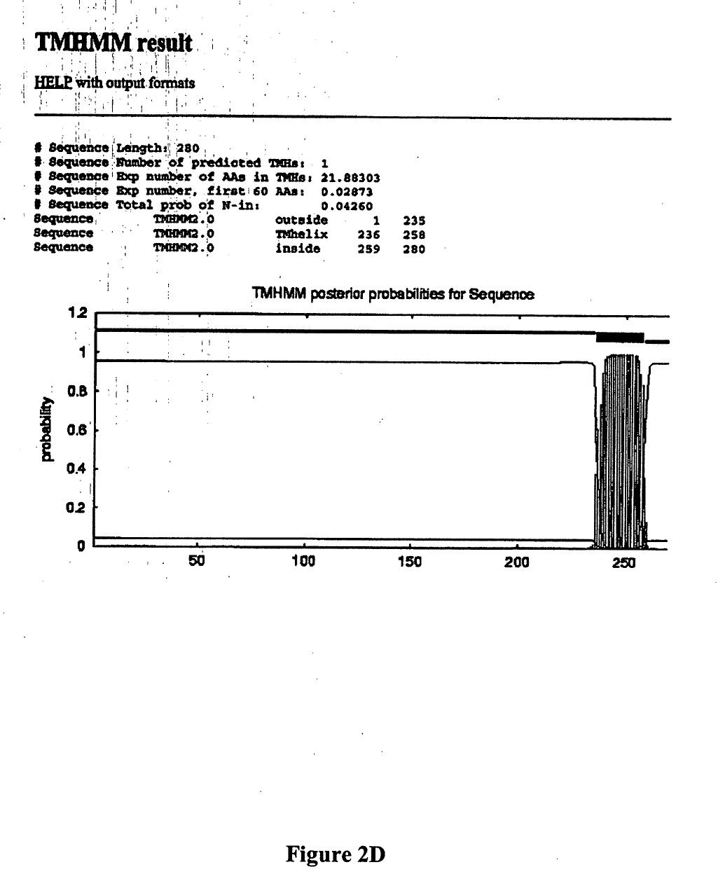

The expression levels of C20orf103 in tumor and control tissue samples are shown in FIGS. 2A-2C. The TMHMM profile of a C20orf103 protein is illustrated in FIG. 2D. The median expression levels of C20orf103 in breast cancer samples, esophageal cancer samples, and lung cancer samples are at least 4.5-, 48-, and 27-fold, respectively, higher than those in the corresponding normal tissue samples. Hence, C20orf103 can be a diagnostic marker and drug target for breast, esophageal, and lung cancers.

CACNA1D encodes calcium channel, voltage-dependent, L type, alpha 1D subunit. Voltage-dependent calcium channels (VDCCs) are multimeric complexes composed of a pore-forming alpha 1 subunit together with several accessory subunits, including alpha 2, delta, beta, and, in some cases, gamma subunits. A family of VDCCs known as the L-type channels are formed specifically from alpha(1S) (skeletal muscle), alpha(1C) (in heart and brain), alpha(1D) (mainly in brain, heart, and endocrine tissue), and alpha(1F) (retina). Neuroendocrine L-type currents have a significant role in the control of neurosecretion and can be inhibited by GTP-binding (G-) proteins.

In cultured rat dorsal root ganglion (DRG) neurons and guinea-pig cardiac myocytes, it was found that the amino acids 1417-1434 of the CACNA1D protein are exposed to the extracellular face of the membrane following depolarization and that the binding of an antibody to these amino acids attenuates calcium channel current.

CACNA1D expression is down-regulated in rat pheochromocytoma (PC12) cells following nerve growth factor treatment, but is up-regulated in the neuropil of the dentate gyrus molecular layer in epilepsy specimens and in cerebral cortical neurons after long term exposure to nicotine. Variations of the CACNA1D may also play a role in the pathogenesis of certain subsets of type 2 diabetes.

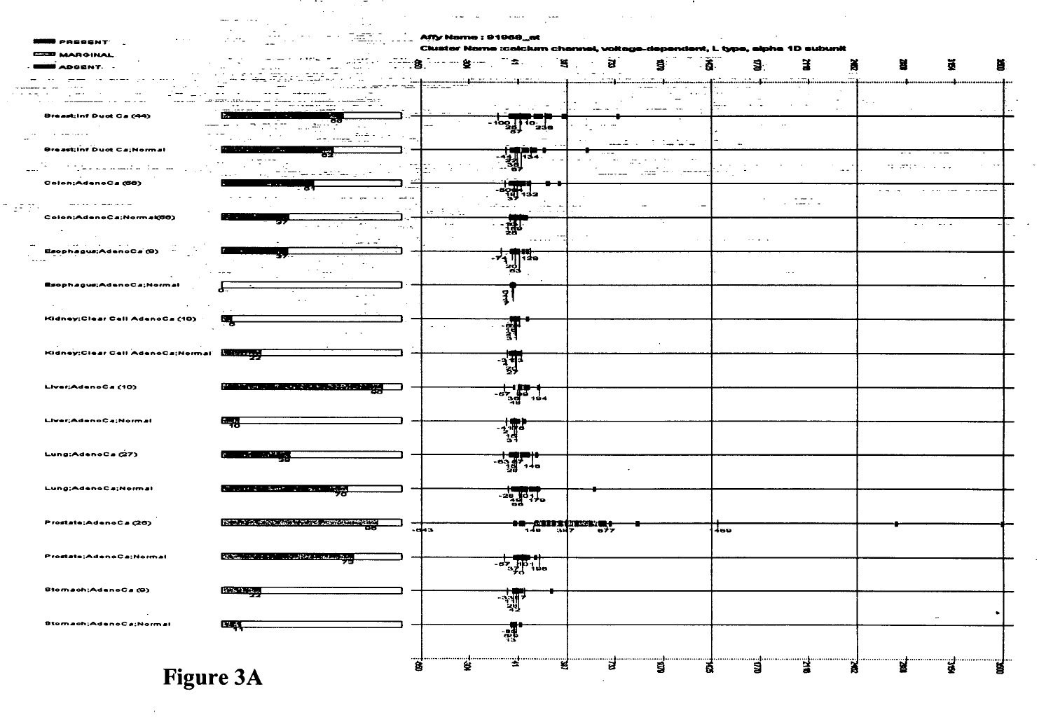

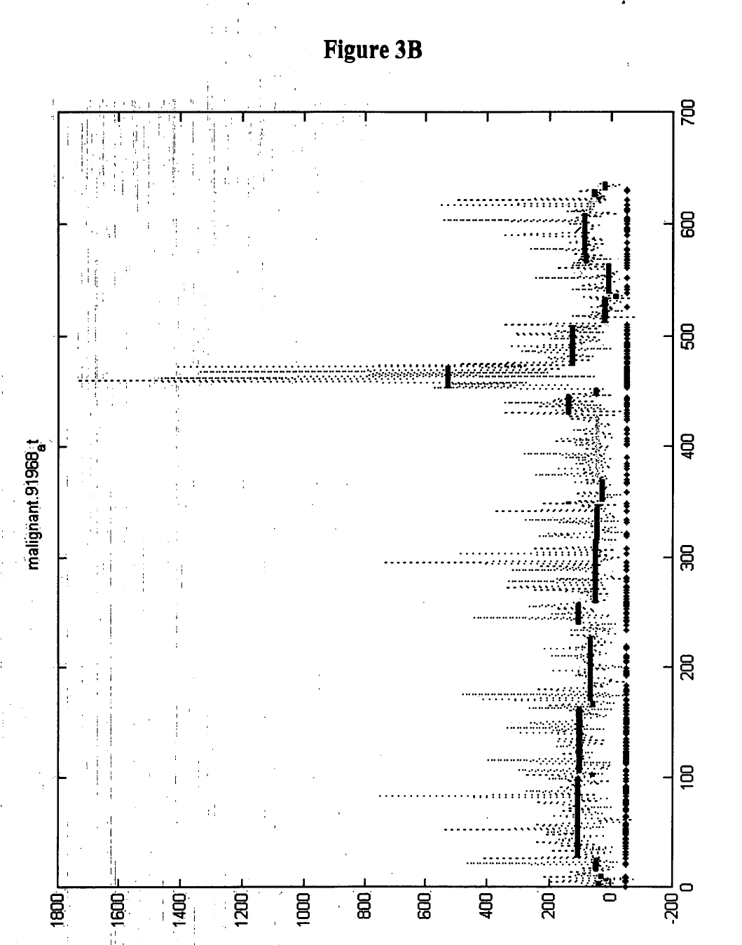

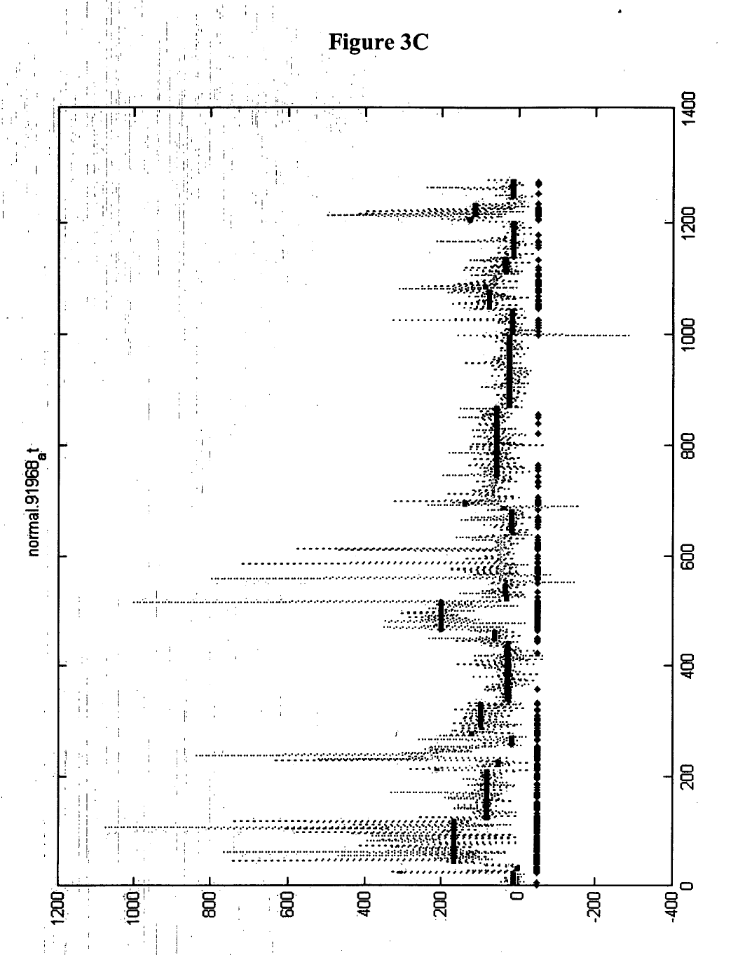

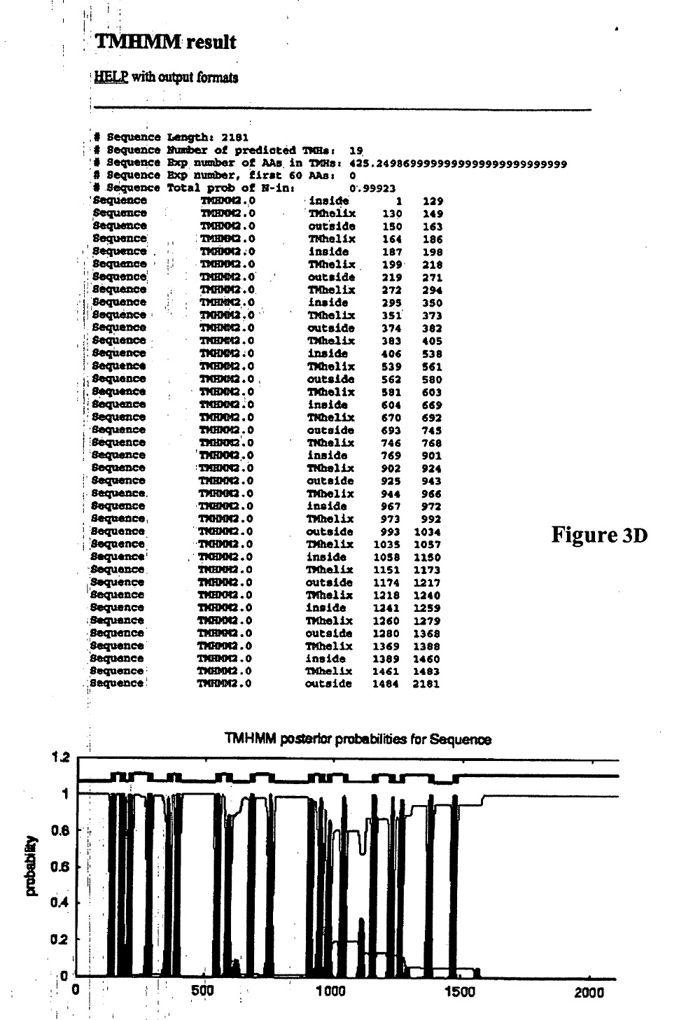

The expression levels of CACNA1D in tumor and control tissue samples are shown in FIGS. 3A-3C. The TMHMM profile of a CACNA1D protein is illustrated in FIG. 3D. The median expression level of CACNA1D in prostate cancer samples is at least 5.5-fold higher than that in its corresponding normal prostate samples. Thus, CACNA1D can be a diagnostic marker and drug target for prostate cancer. CACNA1D can also be a diagnostic marker and drug target for colon, liver, and esophagus cancers.

CDH6 (cadherin 6, type 2, K-cadherin (fetal kidney)) encodes a type II classical cadherin from the cadherin superfamily. The encoded membrane protein is a calcium dependent cell-cell adhesion glycoprotein comprised of five extracellular cadherin repeats, a transmembrane region and a highly conserved cytoplasmic tail. Cadherins mediate cell-cell binding in a homophilic manner, contributing to the sorting of heterogeneous cell types and the maintenance of orderly structures such as epithelium. Strong transcriptional expression of CDH has been observed in hepatocellular carcinoma, small cell lung carcinoma, and renal carcinoma cell lines, suggesting a possible role in metastasis and invasion. Mutational analysis also revealed homozygous deletions of CDH6 on 5p in the SK-LU-1 lung carcinoma cell line (Teng et al., Genomics 74:352-64, 2001).

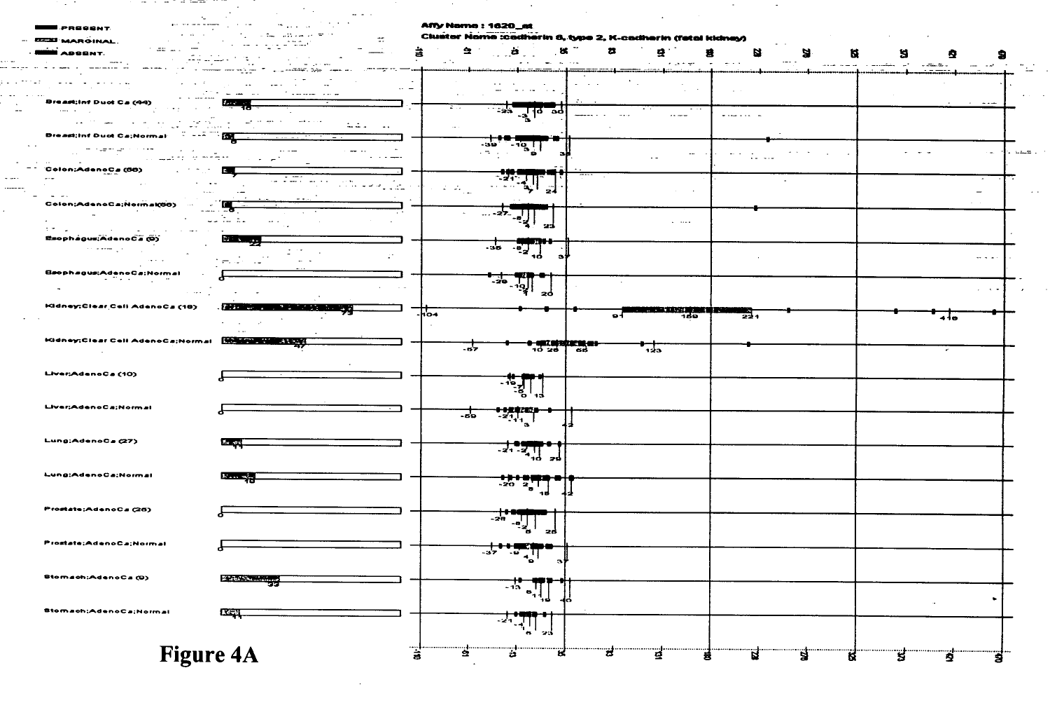

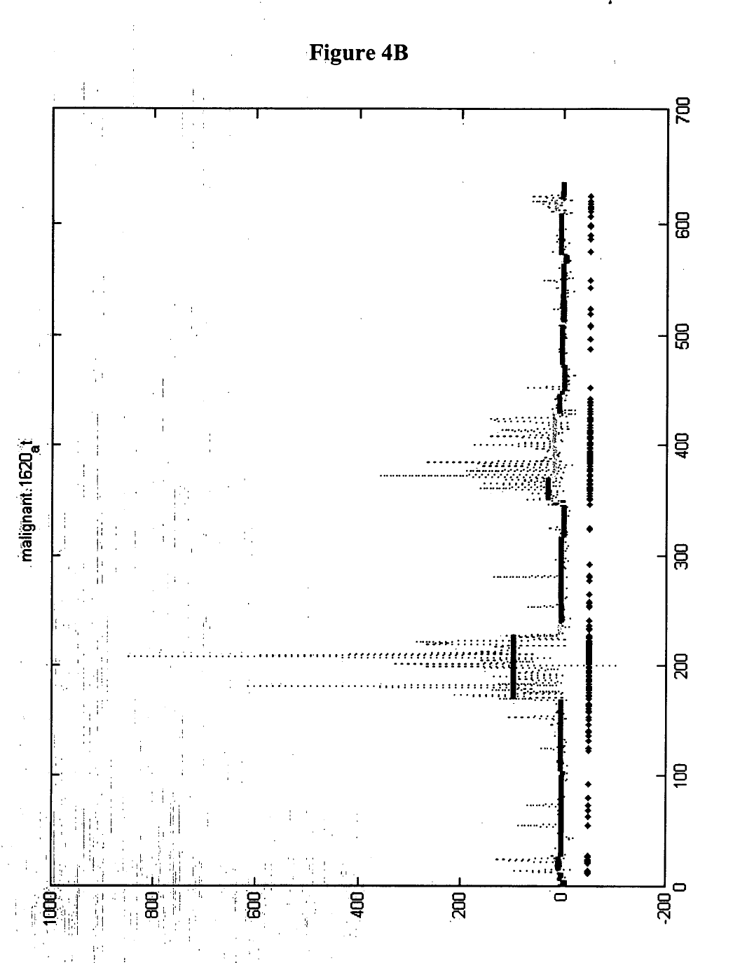

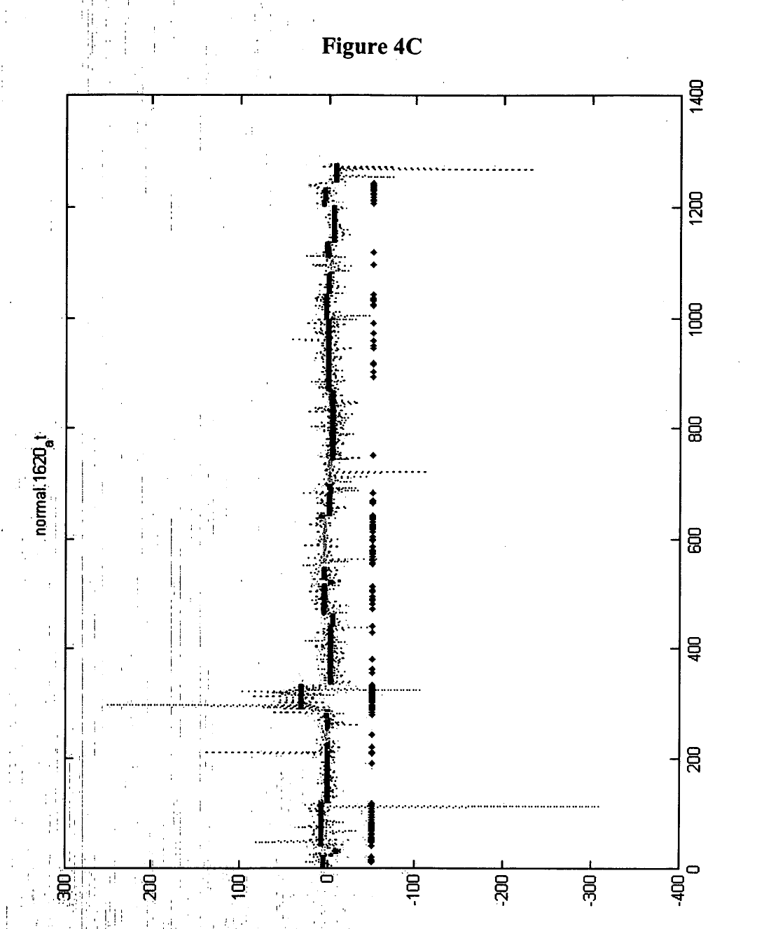

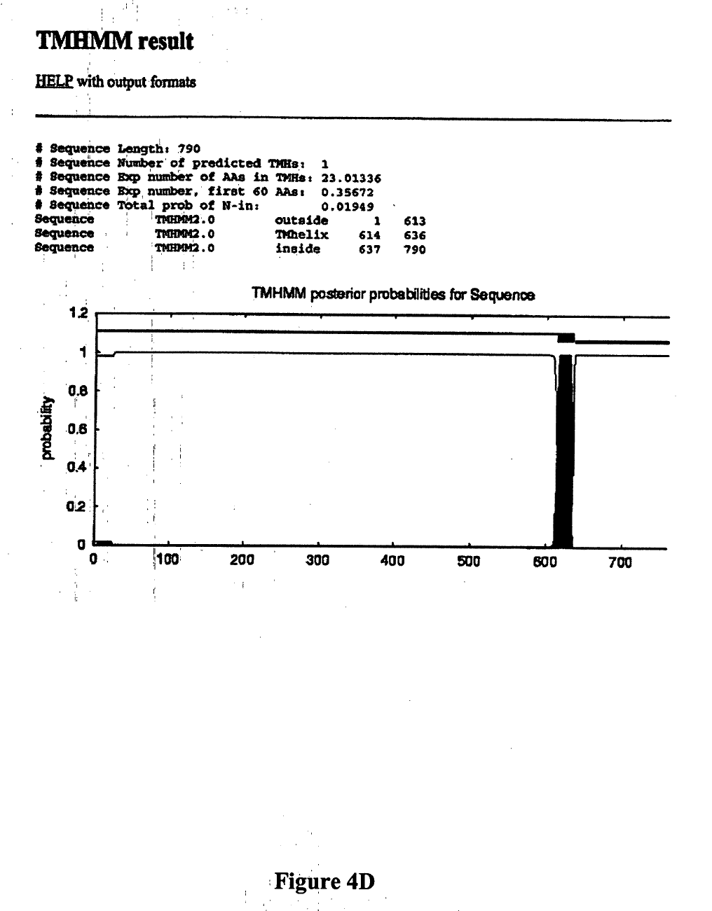

The expression levels of CDH6 in tumor and control tissue samples are shown in FIGS. 4A-4C. The TMHMM profile of a CDH6 protein is illustrated in FIG. 4D. The median expression level of CDH6 in kidney cancer samples is at least 6.1-fold higher than that in its corresponding normal kidney samples. Thus, CDH6 can be a diagnostic marker and drug target for kidney cancer. CDH6 may also be a diagnostic marker and drug target for stomach cancer.

CST encodes cerebroside (3′-phosphoadenylylsulfate:galactosylceramide 3′) sulfotransferase. Sulfonation, an important step in the metabolism of many drugs, xenobiotics, hormones and neurotransmitters, is catalyzed by sulfotransferases. The CST cDNA encodes a 423-amino acid protein with a predicted type II transmembrane topology and 2 potential N-linked glycosylation sites; it is not homologous to either the cytosolic sulfotransferases or the Golgi sulfotransferases. Galactosylceramide sulfotransferase catalyzes the conversion of 3′-phosphoadenylylsulfate and galactosylceramide to adenosine 3′,5′-bisphosphate and galactosylceramide sulfate. CST activity is enhanced in renal cell carcinoma (RCC) by the action of epidermal growth factor, transforming growth factor, and hepatocyte growth factor. It has been suggested that the CST gene is generally over-expressed in human RCE cells via a signaling pathway involving protein kinase-C and tyrosine kinases (Honker et al., Cancer Res., 58:3800-5, 1998). The role of this enzyme in RCE, however, is currently unknown.

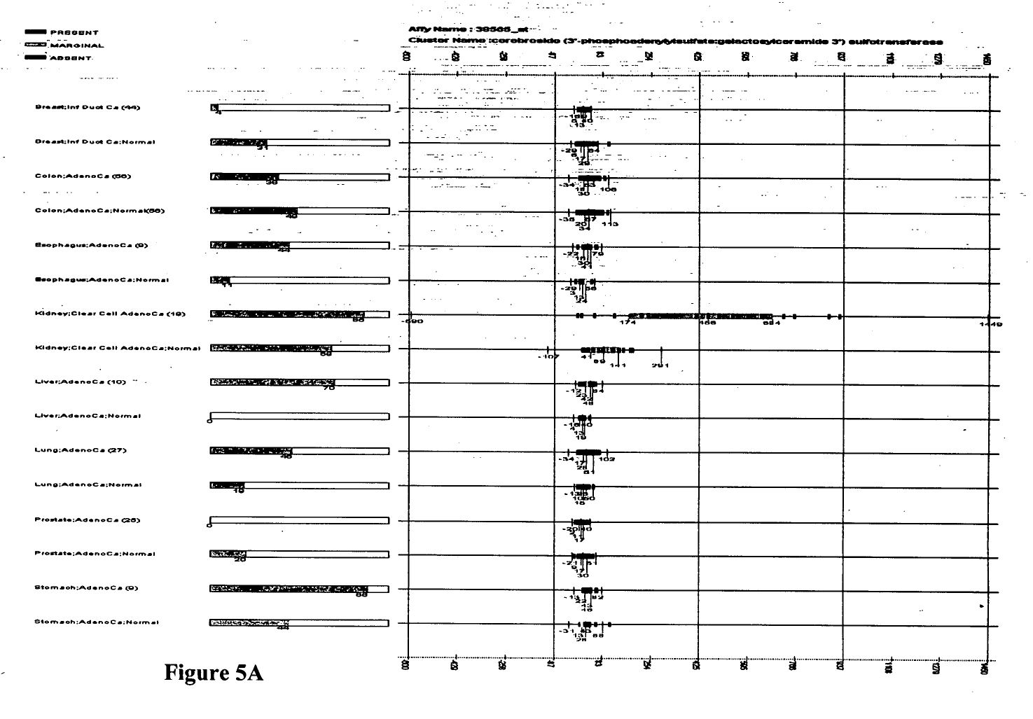

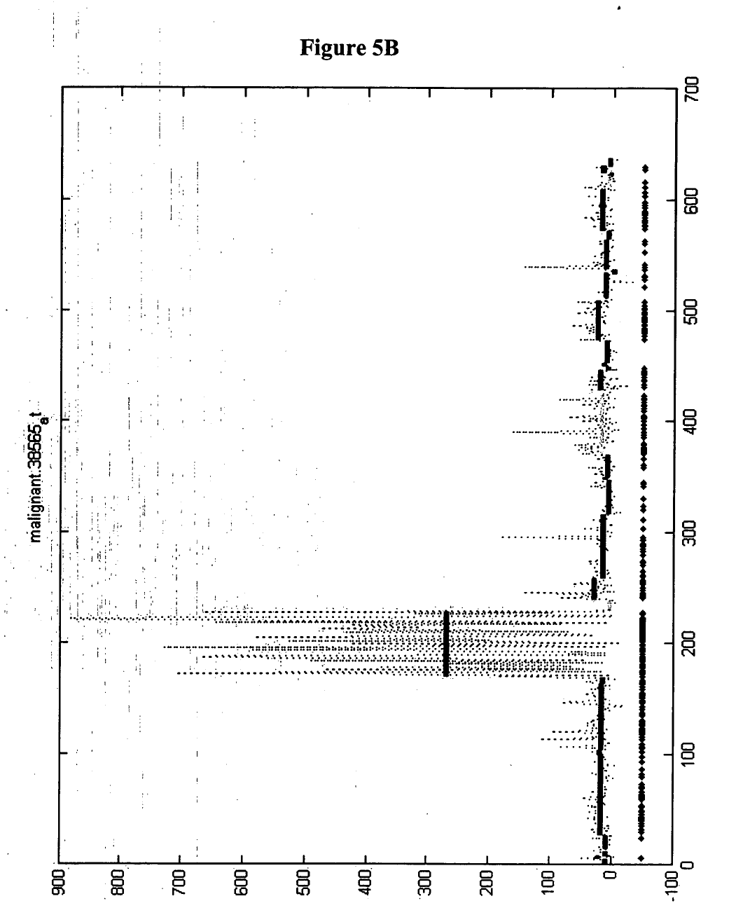

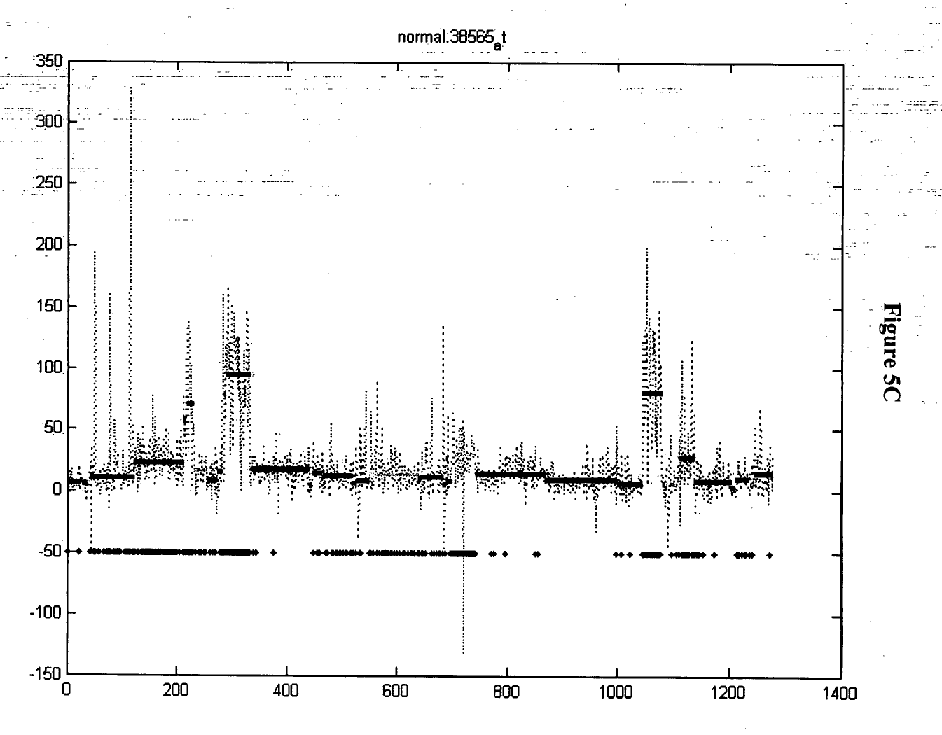

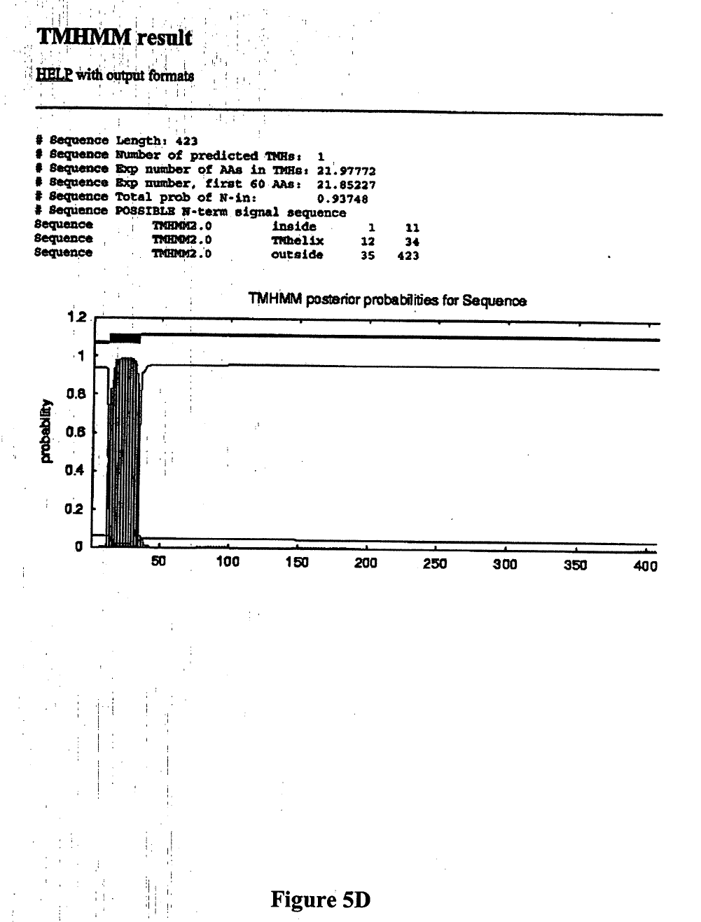

The expression levels of CST in tumor and control tissue samples are shown in FIGS. 5A-5C. The TMHMM profile of a CST protein is illustrated in FIG. 5D. The median expression level of CST in kidney and liver cancer samples is at least 5.1- and 3.2-fold, respectively, higher than those in their corresponding normal kidney and liver samples. Thus, CST can be a diagnostic marker and drug target for both types of cancers. CST may also be a diagnostic marker and drug target for lung and esophagus cancers.

ENPP3 encodes ectonucleotide pyrophosphatase/phosphodiesterase 3. The protein belongs to the family of nucleotide pyrophosphatases/phosphodiesterases (NPPs). NPPs release nucleoside 5′-monophosphates from nucleotides and their derivatives. They exist both as membrane proteins, with an extracellular active site, and as soluble proteins in body fluids. Well-characterized ENPPs include the mammalian ecto-enzymes ENPP1 (PC-1), ENPP2 (autotaxin) and ENPP3 (B10; gp130(RB13-6)). These are modular proteins including a short N-terminal intracellular domain, a single transmembrane domain, two somatomedin-B-like domains, a catalytic domain, and a C-terminal nuclease-like domain. The catalytic domain of ENPPs is conserved from prokaryotes to mammals and shows remarkable structural and catalytic similarities with the catalytic domain of other phospho-/sulfo-coordinating enzymes such as alkaline phosphatases. Hydrolysis of pyrophosphate/phosphodiester bonds by ENPPs occurs via a nucleotidylated threonine. ENPPs are also known to auto(de)phosphorylate this active-site threonine, a process accounted for by an intrinsic phosphatase activity, with the phosphorylated enzyme representing the catalytic intermediate of the phosphatase reaction. ENPP1, ENPP2, and ENPP3 have been implicated in various processes, including bone mineralization, signaling by insulin and by nucleotides, and the differentiation and motility of cells. While it has been established that most of these biological effects of ENPPs require a functional catalytic site, their physiological substrates remain to be identified.

Expression of the related rat ENPP3 mRNA has been found in a subset of immature glial cells and in the alimentary tract. The corresponding rat protein has been detected in the pancreas, small intestine, colon, and liver. Human ENPP3 mRNA is expressed in glioma cells, prostate, and uterus. Expression of the human protein has been detected in uterus, basophils, and mast cells.

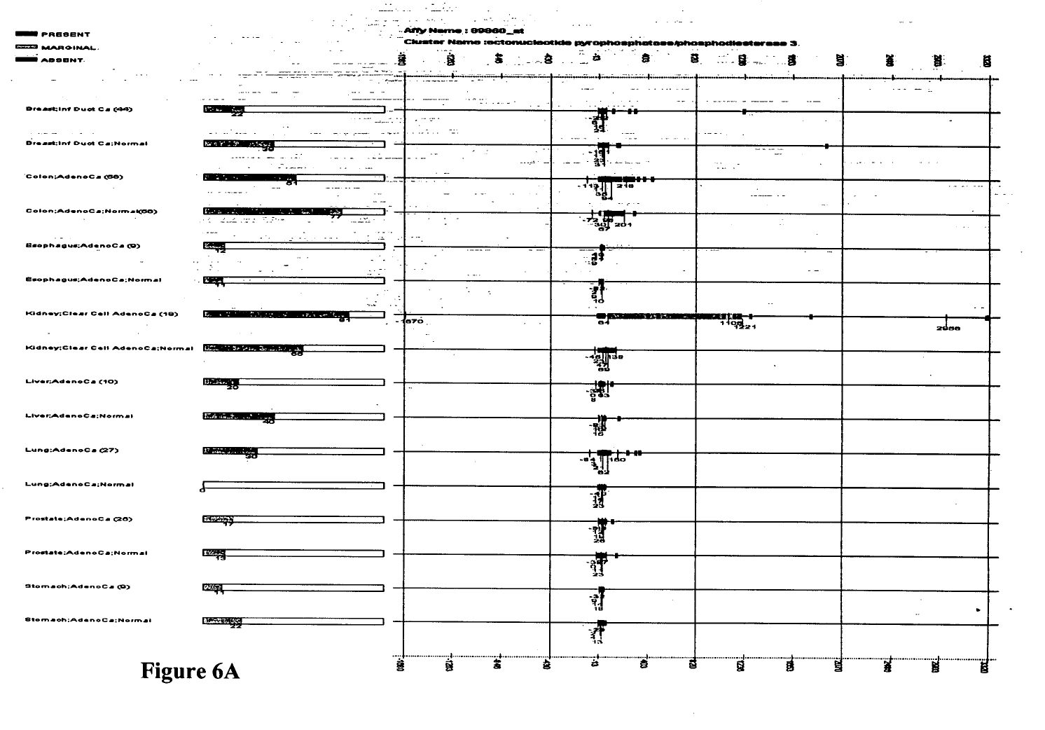

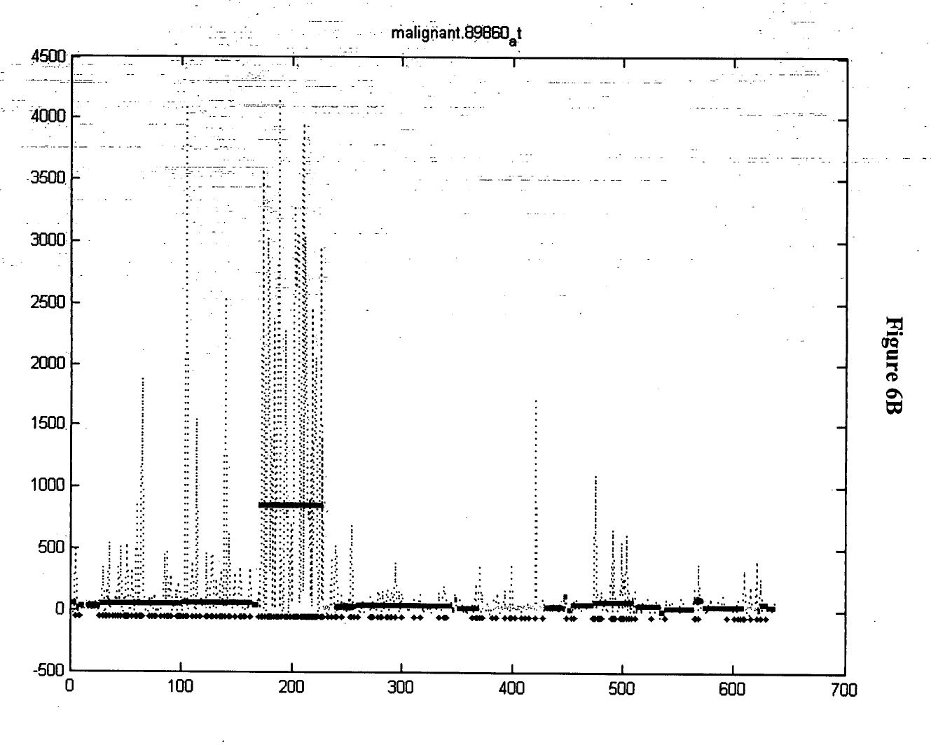

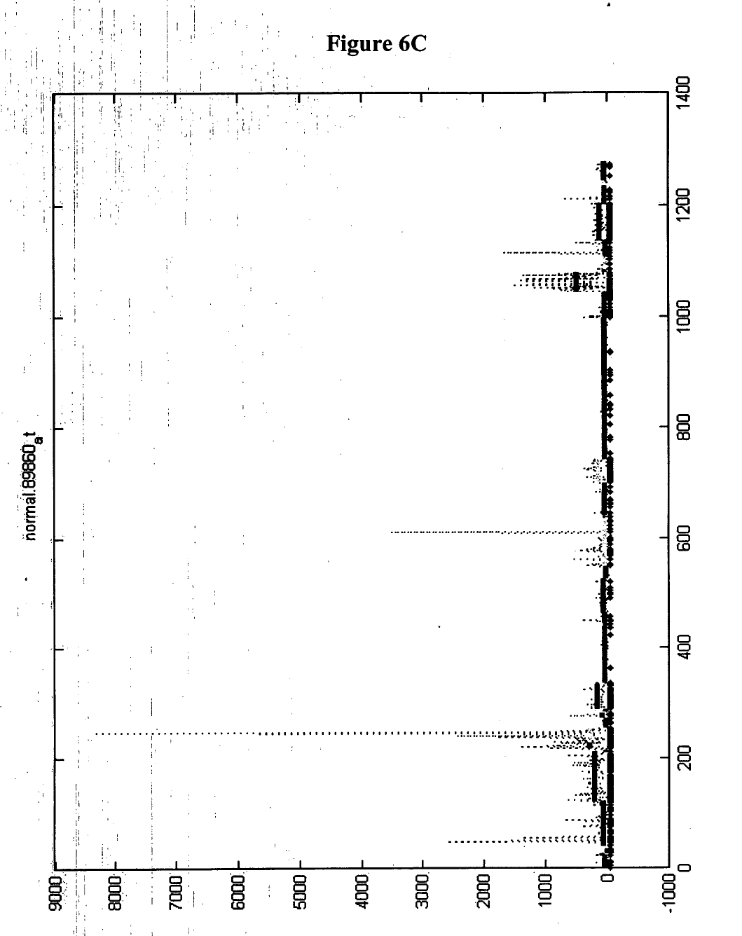

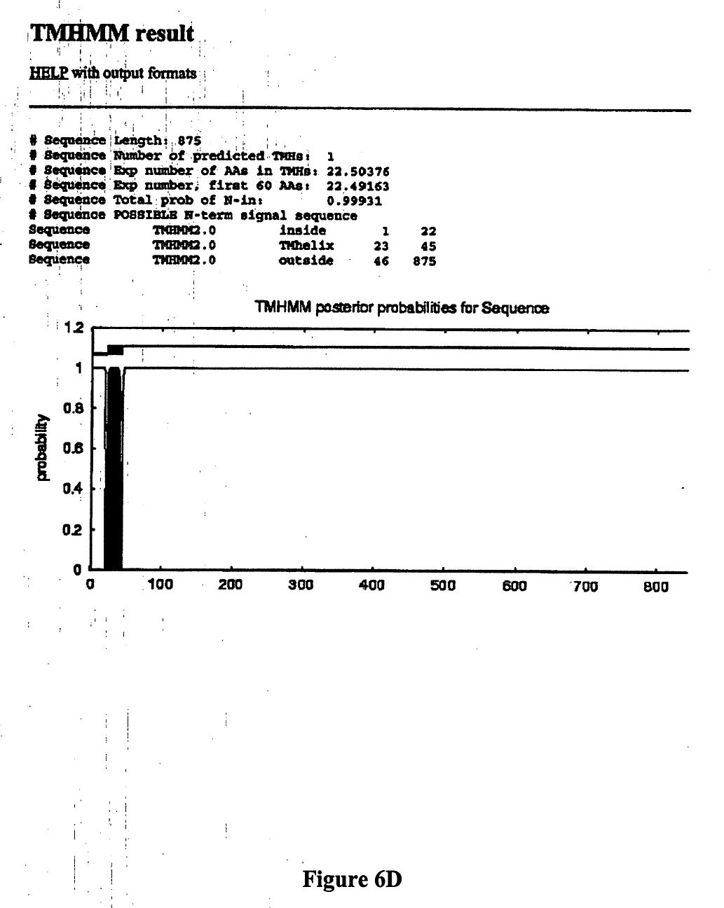

The expression of ENPP3 in tumor and control tissue samples is shown in FIGS. 6A-6C. The TMHMM profile of an ENPP3 protein is illustrated in FIG. 6D. The median expression level of ENPP3 in kidney cancer samples is at least 23-fold higher than that in that the corresponding normal kidney samples. Thus, ENPP3 can be a diagnostic marker and drug target for kidney cancer. ENPP3 may also be a diagnostic marker and drug target for lung cancer.

FLJ11856 (putative G-protein coupled receptor GPCR41), GPR54 (G-protein-coupled receptor 54), and OR51E2 (olfactory receptor, family 51, subfamily E, member 2) all encode G-protein coupled receptors (GPCR). GPCRs, along with G-proteins and effectors (intracellular enzymes and channels which are modulated by G-proteins), are the components of a modular signaling system that connects the state of intracellular second messengers to extracellular inputs. The importance of GPCRs in the etiology of various diseases has been well established. GPCRs and GPCR genes are therefore potential targets for drug action and drug development.

The GPCR protein superfamily now contains over 250 types of paralogues, receptors that represent variants generated by gene duplications (or other processes), as opposed to orthologues, the same receptor from different species. The superfamily can be broken down into five families: Family I, receptors typified by rhodopsin and the beta2-adrenergic receptor and currently represented by over 200 unique members; Family II, the recently characterized parathyroid hormone/calcitonin/secretin receptor family; Family III, the metabotropic glutamate receptor family in mammals; Family IV, the cAMP receptor family, important in the chemotaxis and development of D. discoideum; and Family V, the fungal mating pheromone receptors such as STE2. GPCRs also include receptors for biogenic amines, for lipid mediators of inflammation, peptide hormones, and sensory signal mediators.

GPCR typically has seven transmembrane domains. GPCR becomes activated when the receptor binds its extracellular ligand. Conformational changes in the GPCR, which result from the ligand-receptor interaction, affect the binding affinity of a G protein to the GPCR intracellular domains. This enables GTP to bind with enhanced affinity to the G protein. Activation of the G protein by GTP leads to the interaction of the G protein a subunit with adenylate cyclase or other second messenger molecule generators. This interaction regulates the activity of adenylate cyclase and hence production of a second messenger molecule, cAMP. cAMP regulates phosphorylation and activation of other intracellular proteins. Alternatively, cellular levels of other second messenger molecules, such as cGMP or eicosinoids, may be upregulated or down-regulated by the activity of GPCRs. Activity of GPCR may also be regulated by phosphorylation of the infra- and extracellular domains or loops.

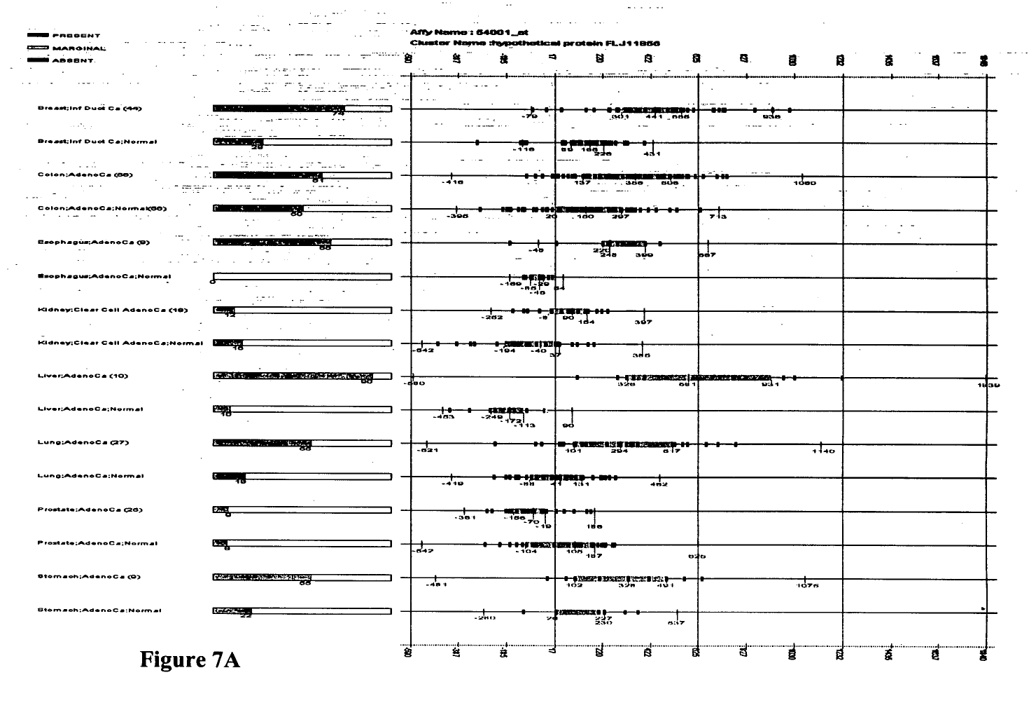

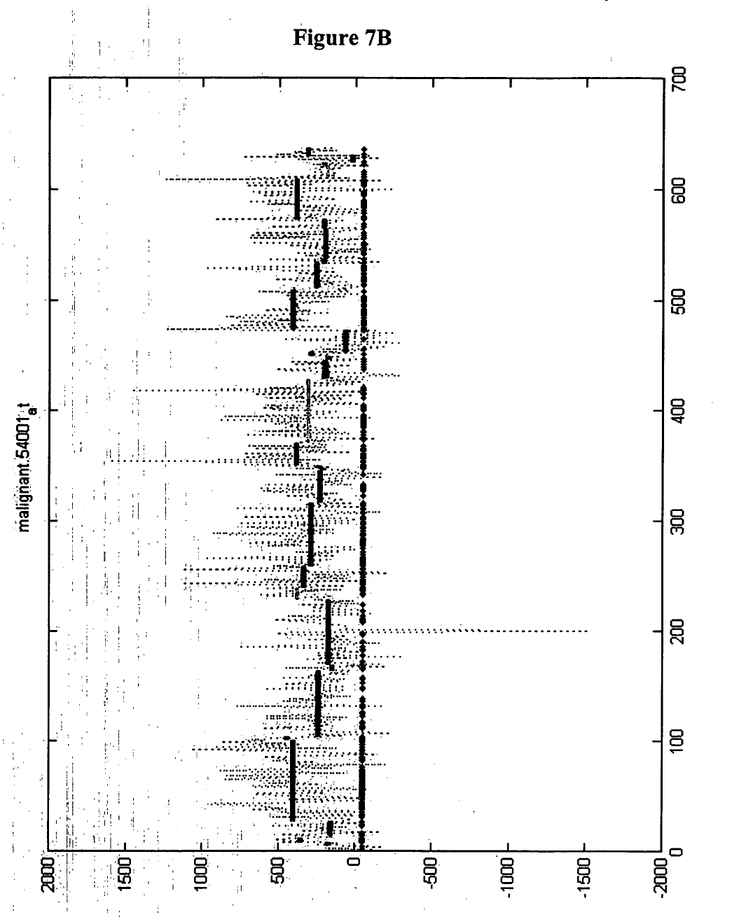

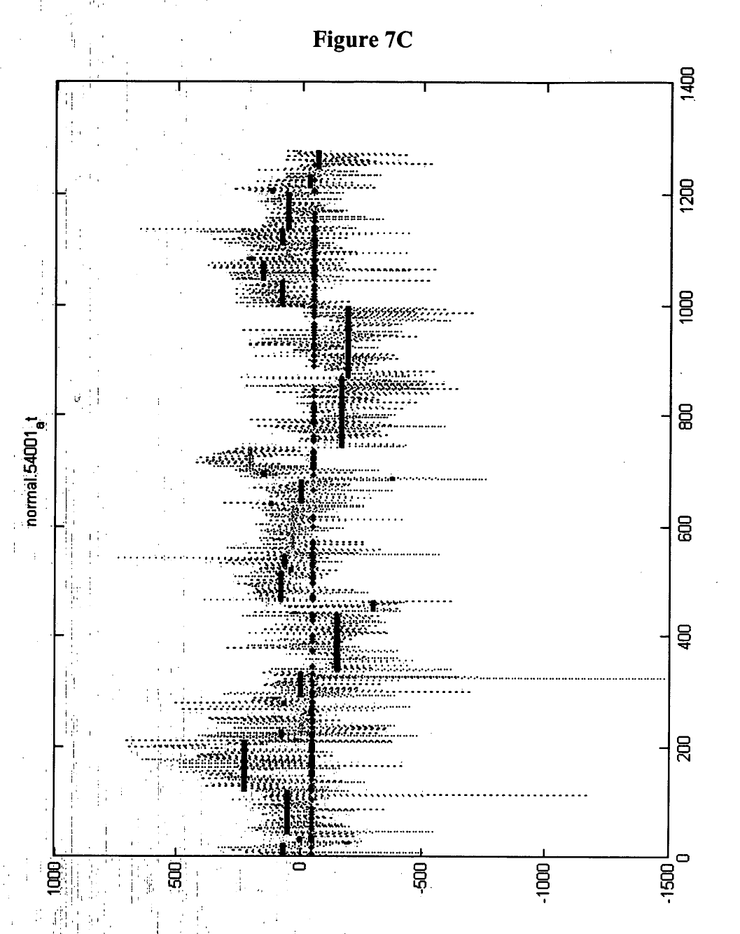

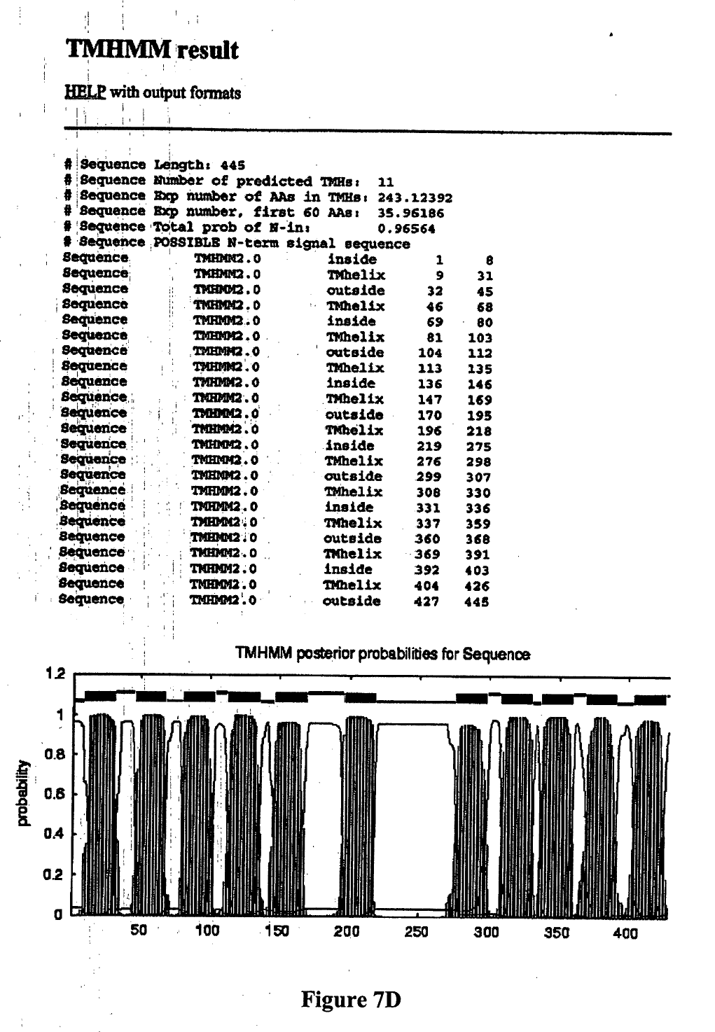

Among the CRTP genes disclosed in the present invention, FLJ11856 encodes a putative GPCR. The function of the protein encoded by FLJ11856 has not yet been determined. The expression of FLJ11856 in tumor and control tissue samples is shown in FIGS. 7A-7C. The TMHMM profile of a FLJ11856 protein is illustrated in FIG. 7D. The median expression levels of FLJ11856 in breast, esophageal, colon, liver, and lung cancer samples is at least 2.7-, 4.5-, 2.7-, 3.4-, and 7.2-fold, respectively, higher than those in the corresponding normal tissue samples. Thus, FLJ11856 can acts as a diagnostic marker and drug target for breast, esophageal, colon, liver, and lung cancers. FLJ11856 may also be a diagnostic marker and drug target for stomach cancer.

GPR54, cloned from a human brain cDNA library, encodes a human GPCR that exhibits 81% homology to the rat orphan receptor GPR54. Heterologous expression of human GPR54 in mammalian cells permitted the identification of three surrogate agonist peptides, all with a common C-terminal amidated motif. High potency agonism, indicative of a cognate ligand, was evident from peptides derived from the gene KiSS-1, the expression of which prevents metastasis in melanoma cells. The highest levels of expression of human GPR54 mRNA were observed in brain, pituitary gland, and placenta. The highest levels of KiSS-1 gene expression were observed in placenta and brain. A polyclonal antibody raised to the C terminus of the human GPR54 showed binding to neurons in the cerebellum, cerebral cortex, and brainstem.

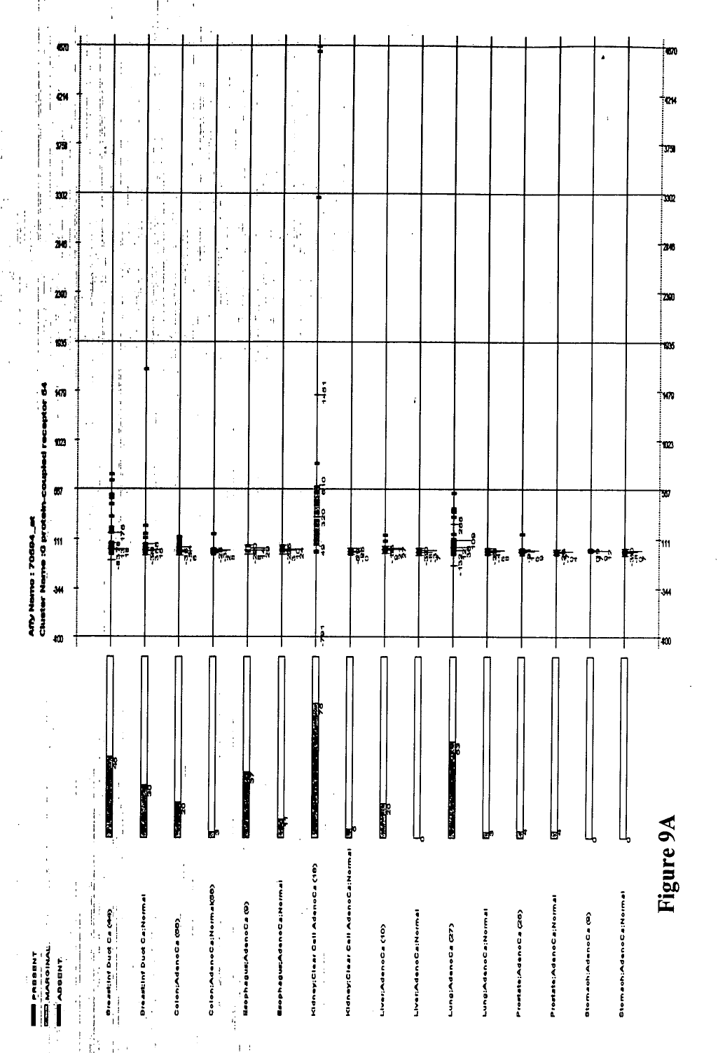

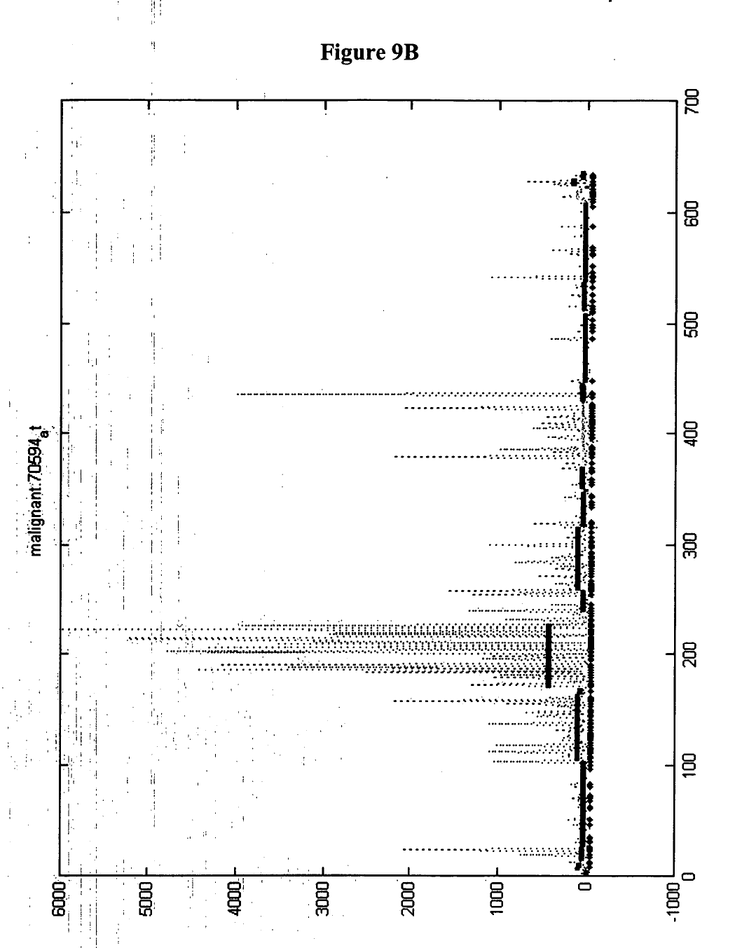

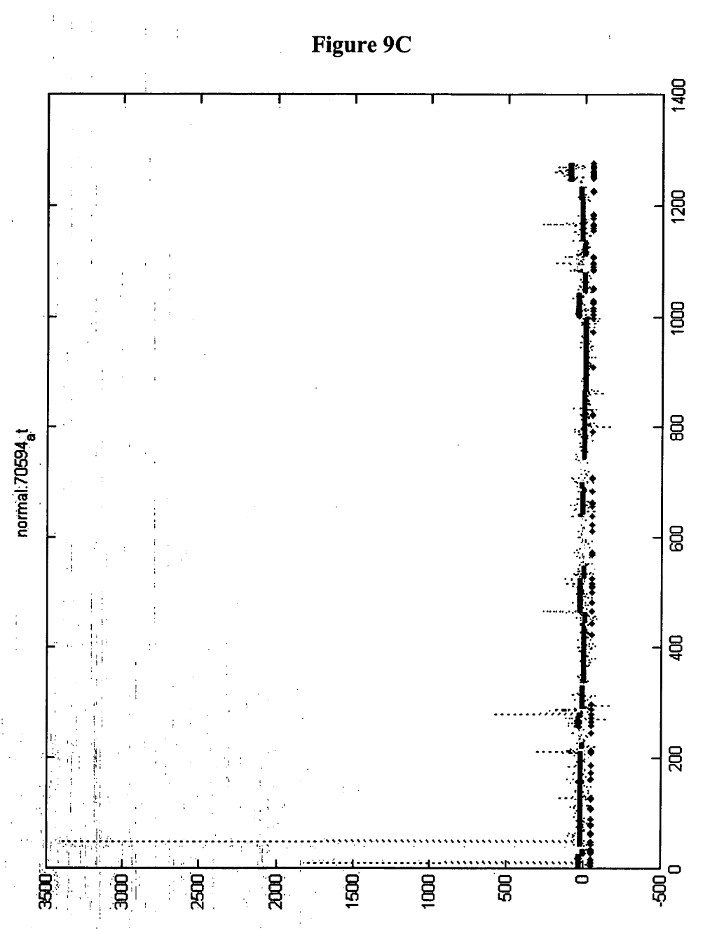

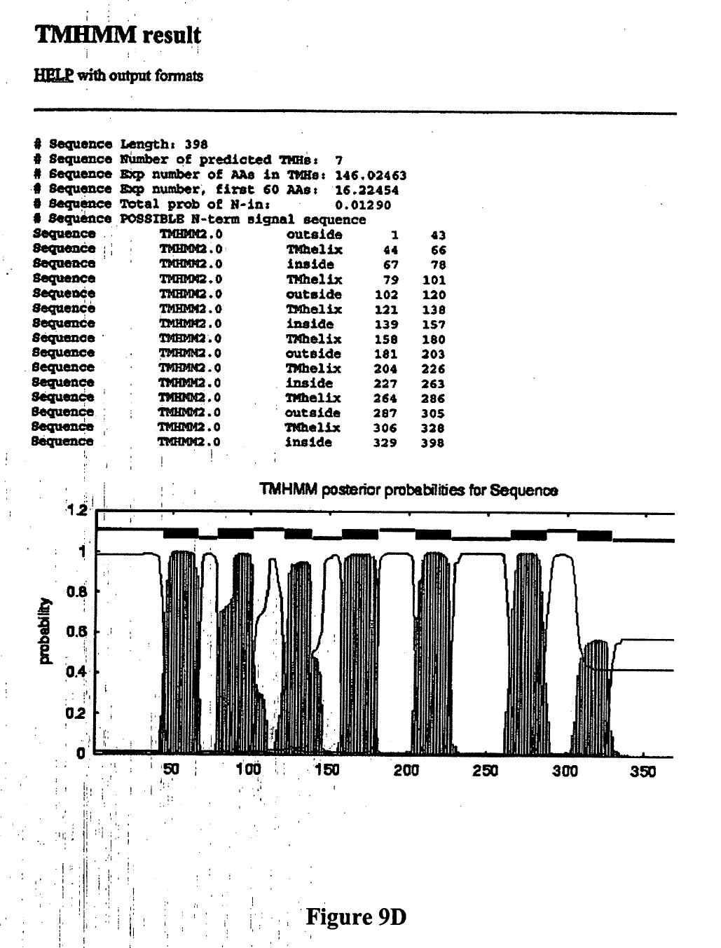

The expression of GPR54 in tumor and control tissue samples is shown in FIGS. 9A-9C. The TMHMM profile of a GPR54 protein is illustrated in FIG. 9D. The median expression levels of GPR54 in kidney and lung cancer samples is at least 12- and 9-fold, respectively, higher than those in the corresponding normal tissue samples. Thus, GPR54 can be a diagnostic marker and drug target for kidney and lung cancers. GPR54 can also be a diagnostic marker and drug target for breast cancer.

OR51E2 encodes a human prostate specific G-protein coupled receptor with properties characteristic of an olfactory receptor. A partial cDNA sequence of this gene, called PSGR, was recently cloned. The gene contains two exons and one intron of 14.9 kb in its 5′ untranslated region, and was mapped to human chromosome 11p15.2. A cluster of transcription initiation sites for the 2.8 kb PSGR mRNA was identified. Cloning of the homologous gene from the mouse revealed 93% amino acid homology between the human and mouse or rat (previously cloned as RA1c) proteins, and 99% identity between the rat and mouse homologs. Although northern analysis indicated expression of the human PSGR homolog was prostate specific, its mRNA could also be detected in the olfactory zone and the medulla oblongata of the human brain. In the mouse, the PSGR gene is predominantly expressed in the brain and colon. In the rat, the PSGR homolog is expressed in the liver in addition to the brain. These data add to the growing body of evidence suggesting that olfactory receptors may have functional roles in tissues other than the olfactory organ, a further, suggest that these functions may vary across species.

A study using multiple tissue Northern blots from over 50 different tissues revealed that OR51E2 expression was restricted to human prostate tissues. The differential expression of OR51E2 between normal and tumor tissues was highly significant (P<0.001), and 32 of 52 (62%) matched prostate specimens exhibited tumor-associated overexpression of OR51E2. There was very little or no expression of OR51E2 in many normal specimens in comparison with the generally high expression of OR51E2 seen in matched tumor specimens. In situ hybridization assays showed restricted OR51E2 expression in the epithelial cells of the normal and tumor tissue sections. See Xu et al., Cancer Res., 60:6568-72, 2000.

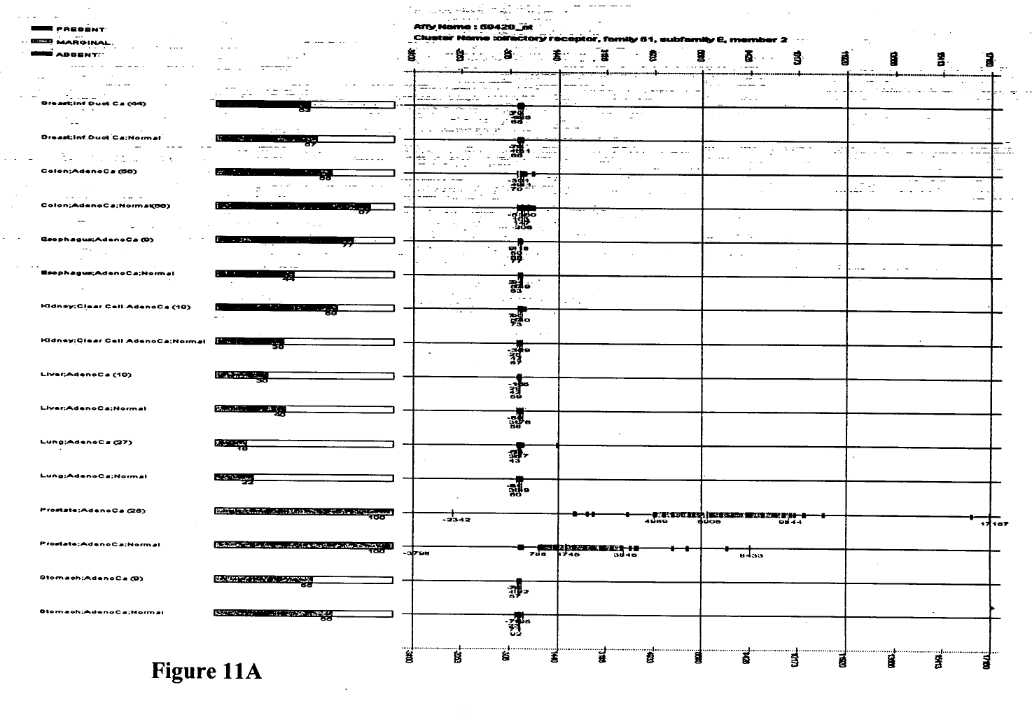

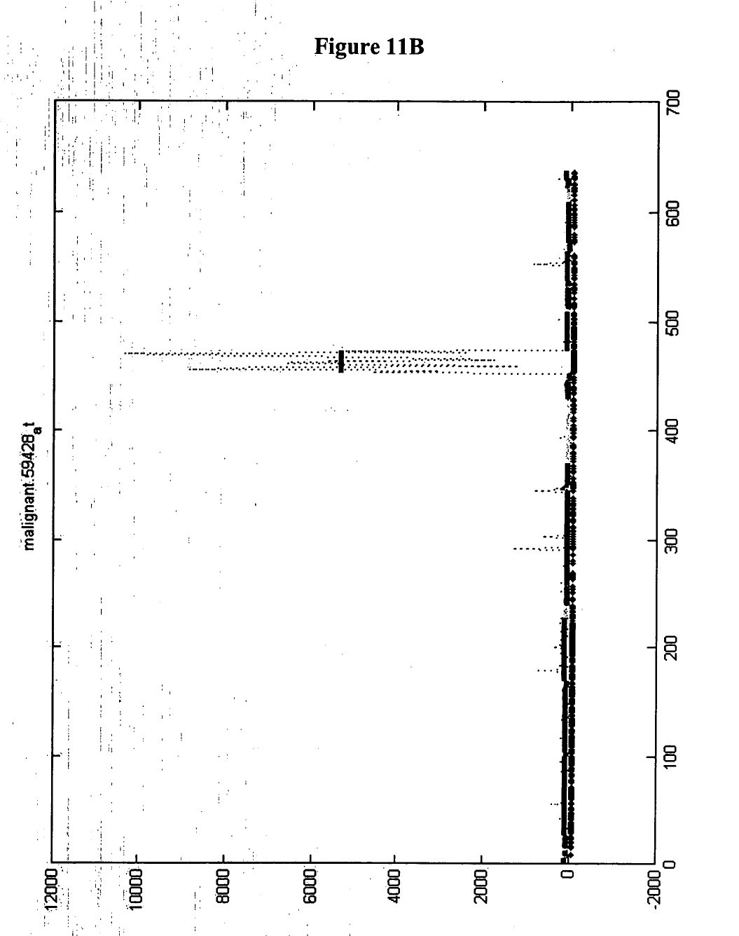

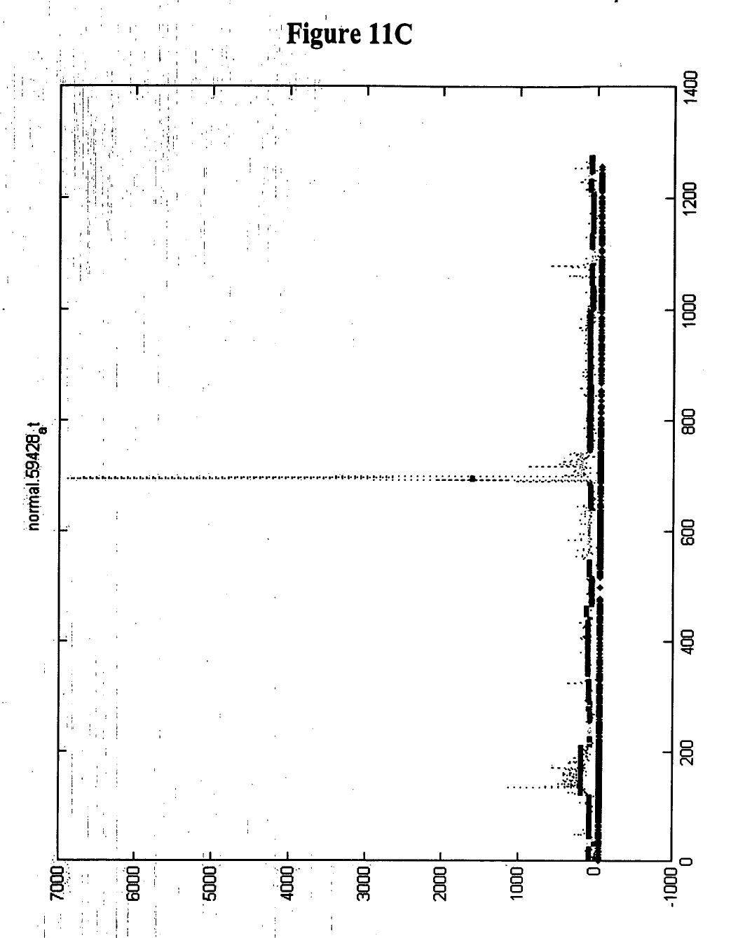

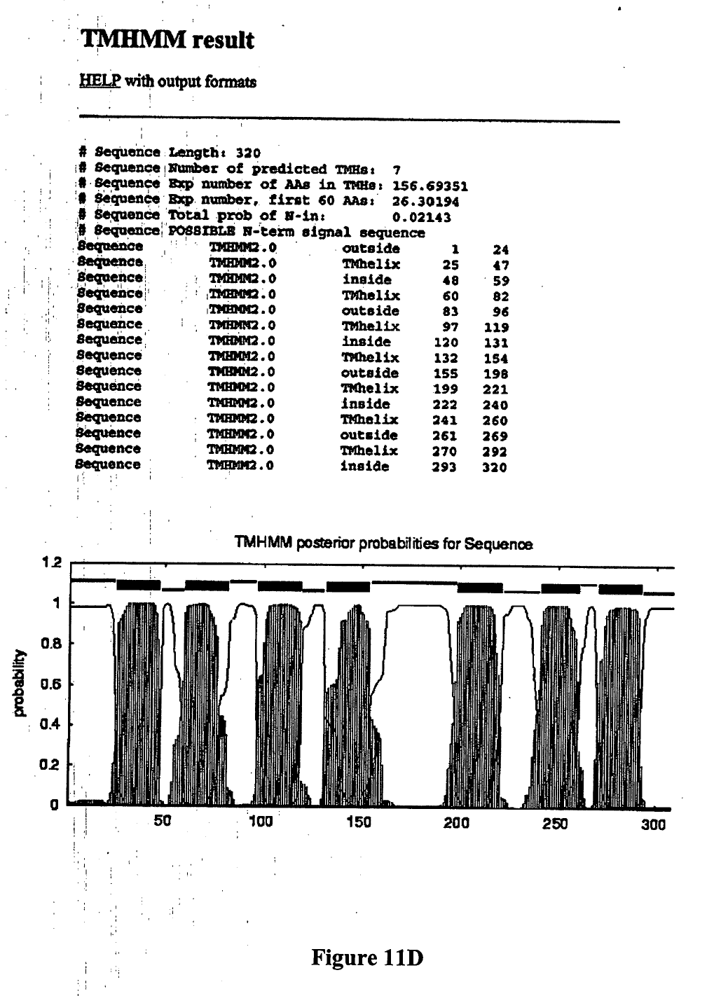

The expression of OR51E2 in tumor and control tissue samples is shown in FIGS. 11A-11C. The TMHMM profile of an OR51E2 protein is illustrated in FIG. 1D. The median expression level of OR15E2 in prostate cancer samples is at least 3.9-fold higher than that in the corresponding normal tissue samples. Thus, OR15E2 may acts as a diagnostic marker and drug target for prostate cancer.

FOLH1 (folate hydrolase 1), also known as prostate-specific membrane antigen 1, encodes an N-acetylated, α-linked acidic dipeptidase which also hydrolyzes folate. FOLH1 is a 750-amino acid type II membrane glycoprotein, which is primarily expressed in normal human prostate epithelium. Expression of FOLH1 is upregulated as prostate tumor grade increases and is found in the vasculature of many tumors, with no or less presence in benign tissues.

cDNA microarray screening of gene expression patterns in benign prostatic hyperplasia BPH and tumor samples indicated that FOLH1 was significantly over-expressed in prostate cancer compared to BPH. Prostate epithelial cells stained positively for FOLH1 in prostate cancer tissues, whereas the majority of BPH tissues showed negative staining. (Burger et al., Int. J. Cancer, 100:228-37, 2002).

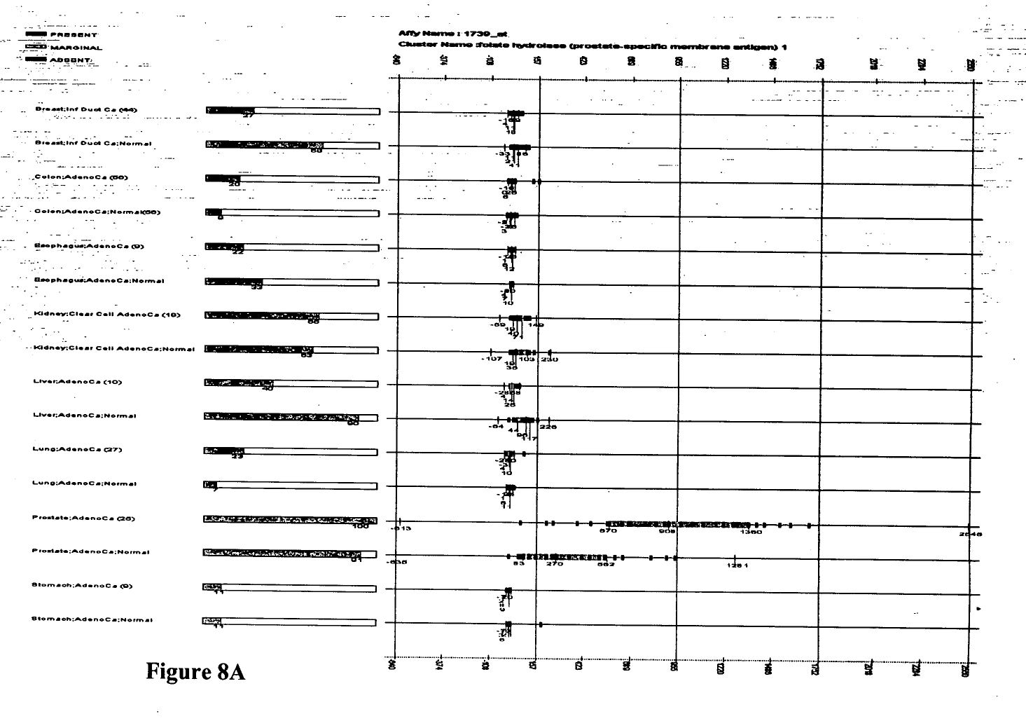

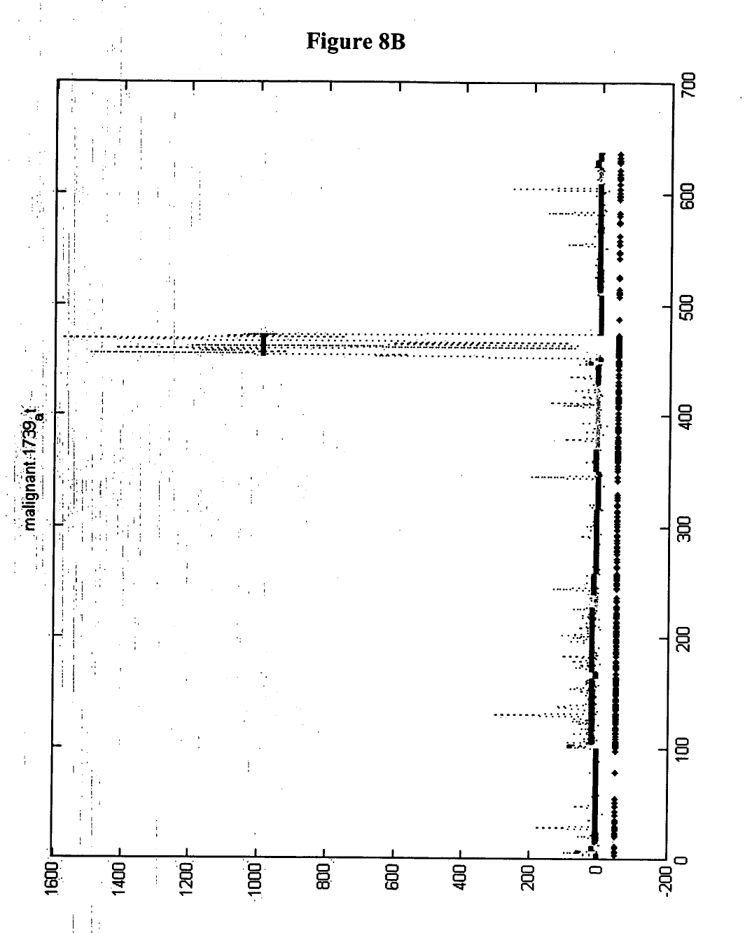

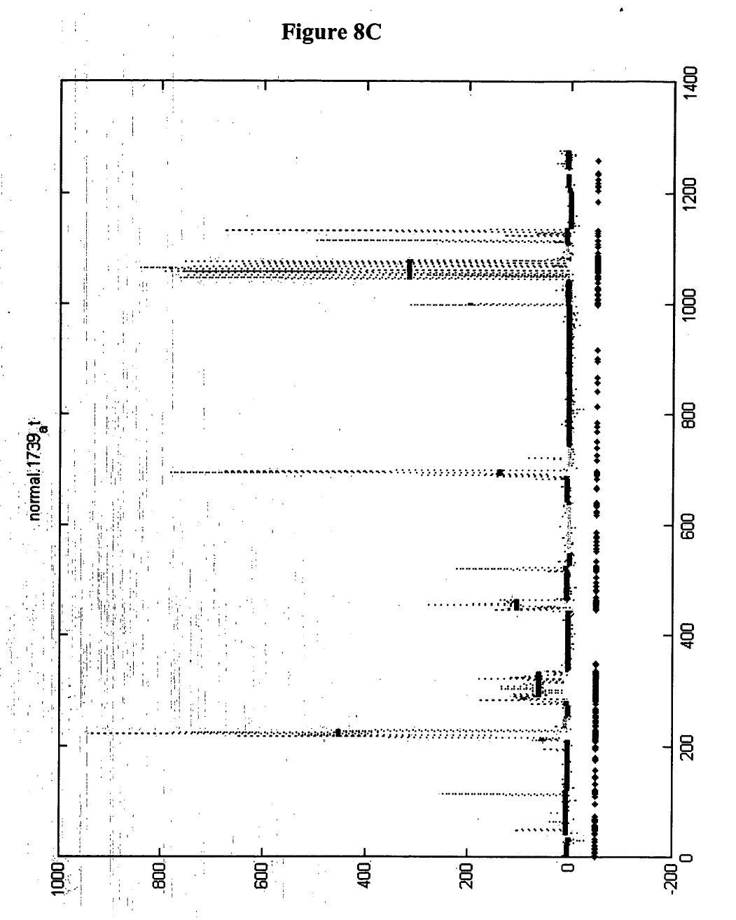

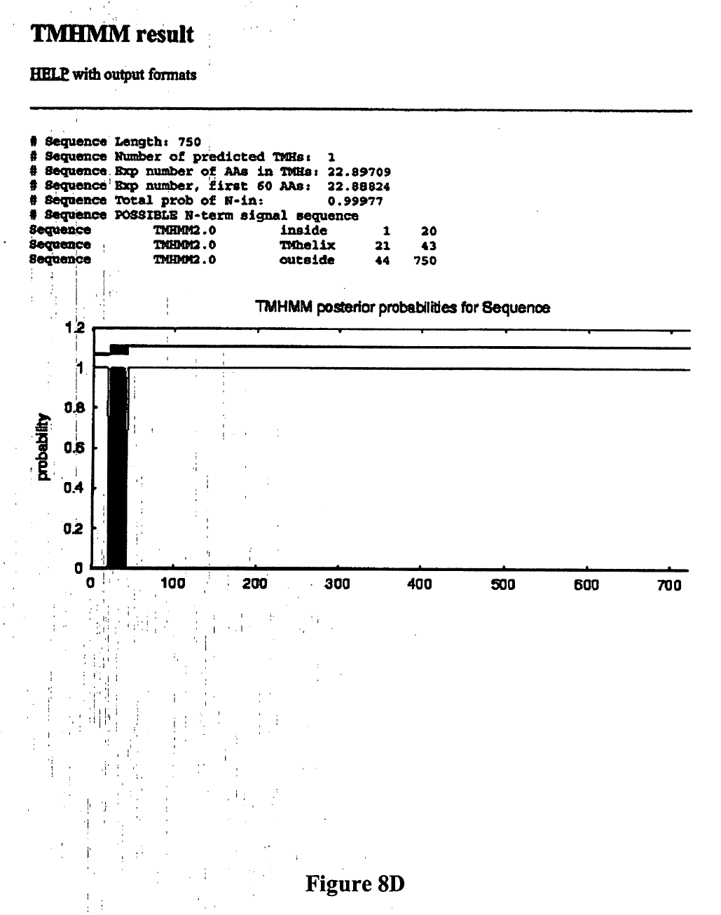

The expression of FOLH1 in tumor and control tissue samples is shown in FIGS. 8A-8C. The TMHMM profile of a FOLH1 protein is illustrated in FIG. 8D. The median expression level of FOLH1 in prostate cancer samples is about 3.4-fold higher than that in the corresponding normal prostate tissue samples. Thus, FOLH1 can be a diagnostic marker and drug target for prostate cancer.

HAVCR1 encodes hepatitis A virus cellular receptor 1. The protein is approximately 79% identical to mouse HAVCR1, which was identified as a receptor for the hepatitis A virus. The six Cys residues of the extracellular domain and the first N-glycosylation site of mouse HAVCR1 are conserved in human HAVCR1. However, the number of hexameric repeats of the mucin-like region is reduced from 27 in mouse HAVCR1 to 13 in human HAVCR1. HAVCR1 is expressed in liver, small intestine, colon, and spleen, and is expressed at higher levels in the kidney and testis. Antibody protection experiment suggests that HAVCR1 is a functional receptor for hepatitis A virus in humans.

The HAVCR1 protein contains an immunoglobulin (Ig) domain and is moderately similar to rat kidney injury molecule-1 (KIM-1). Structurally, KIM-1 is a member of the immunoglobulin gene superfamily most reminiscent of mucosal addressin cell adhesion molecule 1 (MAdCAM-1). It was reported that HAVCR1 mRNA and protein are expressed at a low level in normal kidney but are increased dramatically in post-ischemic kidney. In situ hybridization and immunohistochemistry revealed that HAVCR1 is expressed in proliferating bromodeoxyuridine-positive and dedifferentiated vimentin-positive epithelial cells in regenerating proximal tubules. HAVCR1 may play an important role in the restoration of the morphological integrity and function to post-ischemic kidney.

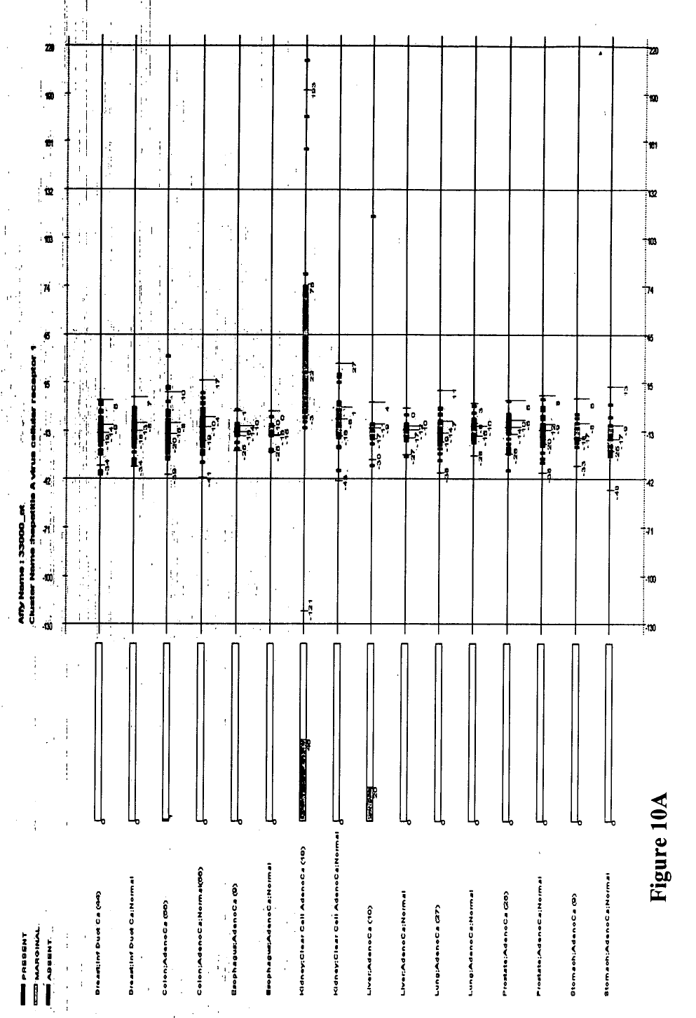

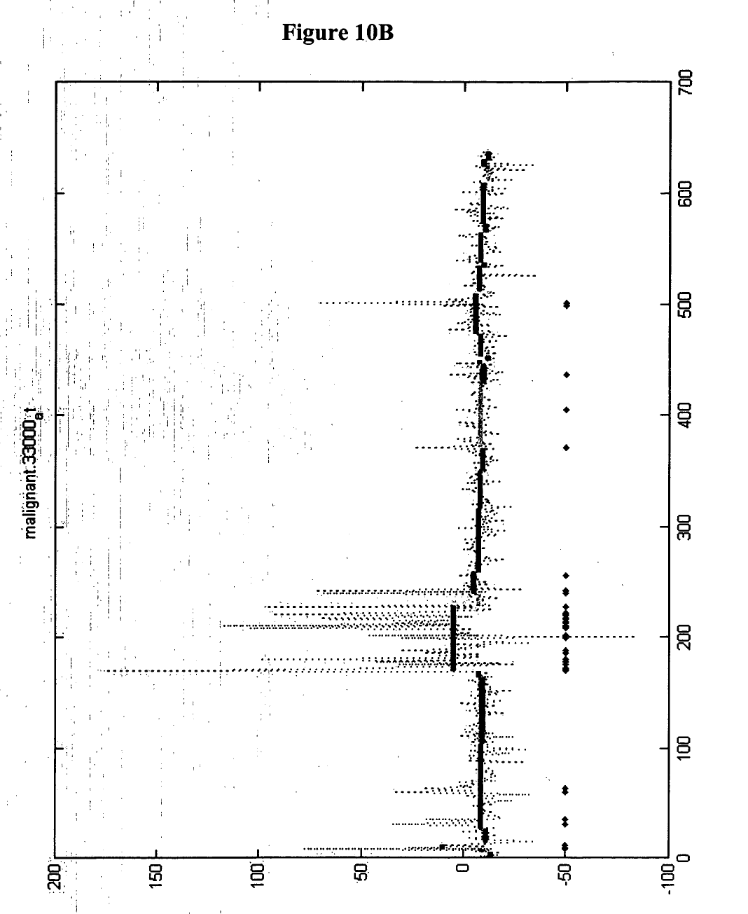

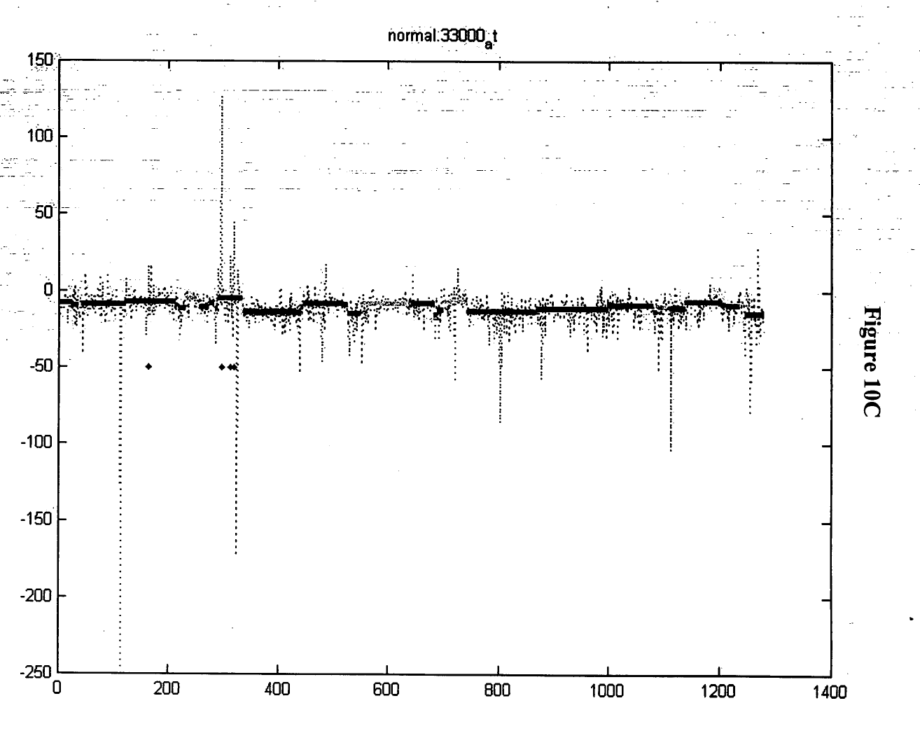

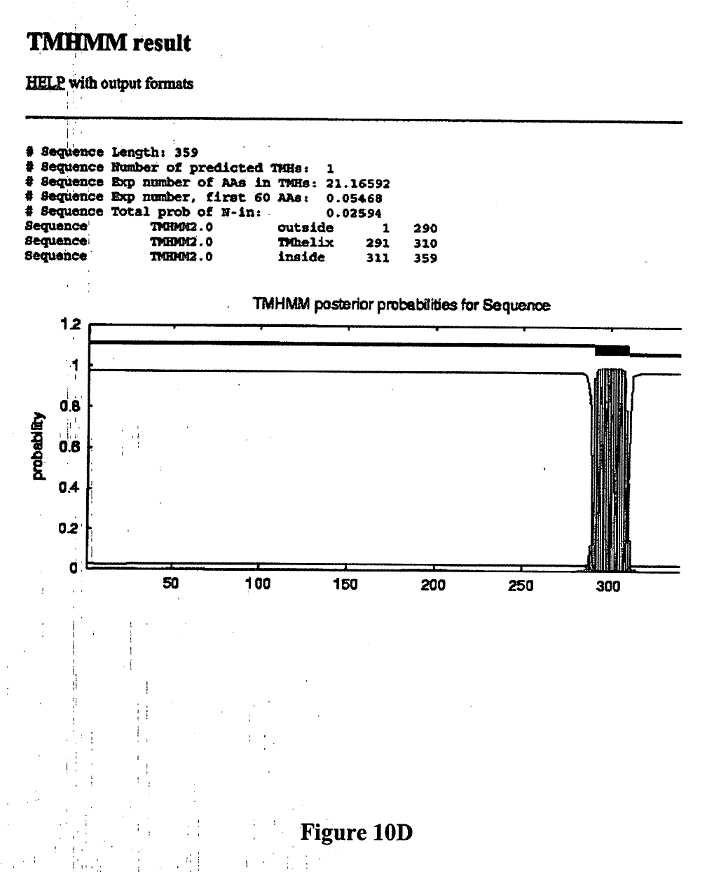

The expression of HAVCR1 in tumor and control tissue samples is shown in FIGS. 10A-10C. The TMHMM profile of a HAVCR1 protein is illustrated in FIG. 10D. The median expression level of HAVCR1 in kidney cancer samples is about 3.7-fold higher than that in the corresponding normal kidney tissue samples. Therefore, HAVCR1 can be a diagnostic marker and drug target for kidney cancer.

SLC6A3 (solute carrier family 6 (neurotransmitter transporter, dopamine), member 3) encodes a dopamine transporter 3 which is strongly similar to murine Dat1. Dopamine transporter is responsible for presynaptic reuptake of dopamine and is a major site of action of psychostimulant drugs, including cocaine. The protein's actions and its specific localization to dopaminergic neurons make it a candidate gene for several psychiatric illnesses. SLC6A3 has been mapped to distal chromosome 5p. Alleles at this locus have been reported to be associated with cocaine-induced paranoia and attention deficit disorder.

The SLC6A3 gene has also been linked to smoking initiation and nicotine dependence, to obesity in African-American smokers, and to the occurrence of posttraumatic stress disorder (PTSD) among trauma survivors. The SLC6A3 gene, however, has not yet been linked to cancer.

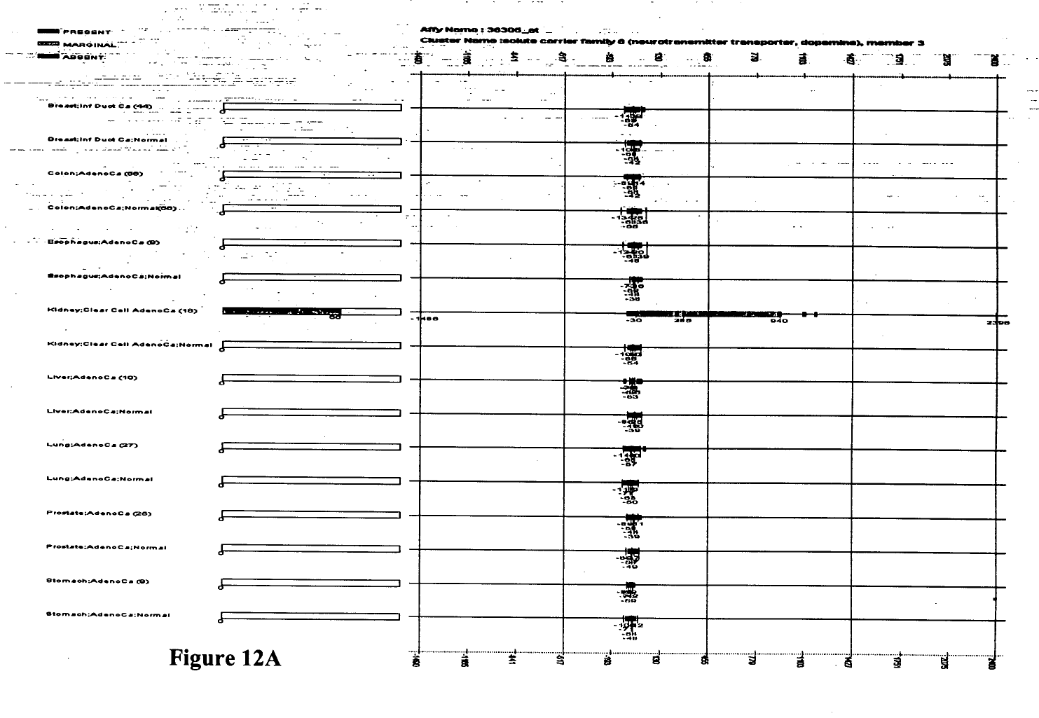

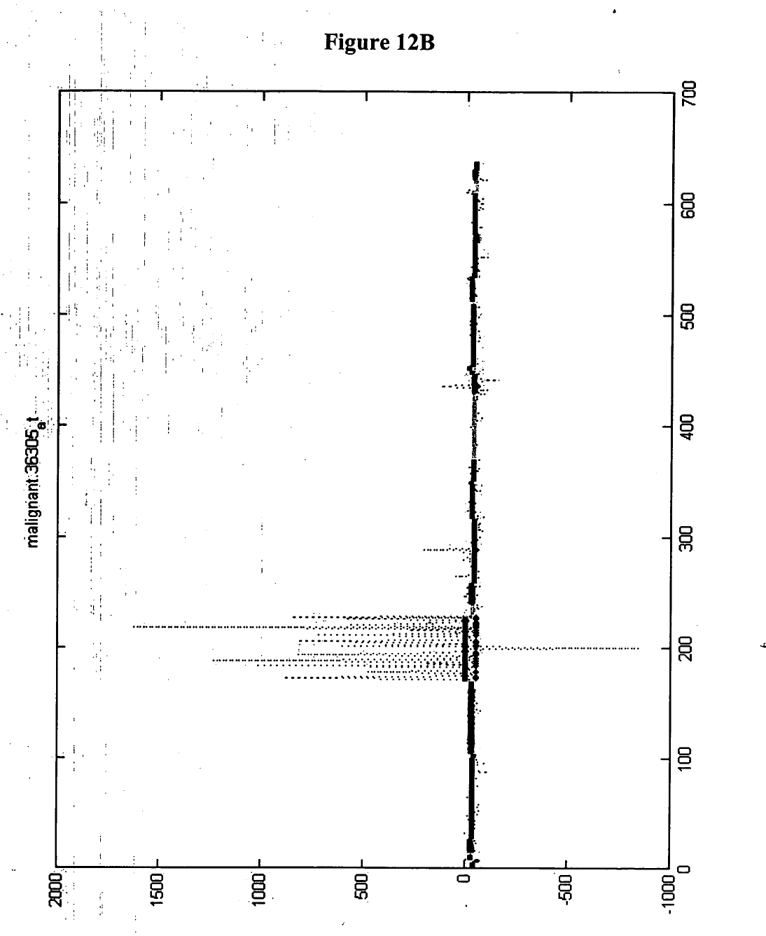

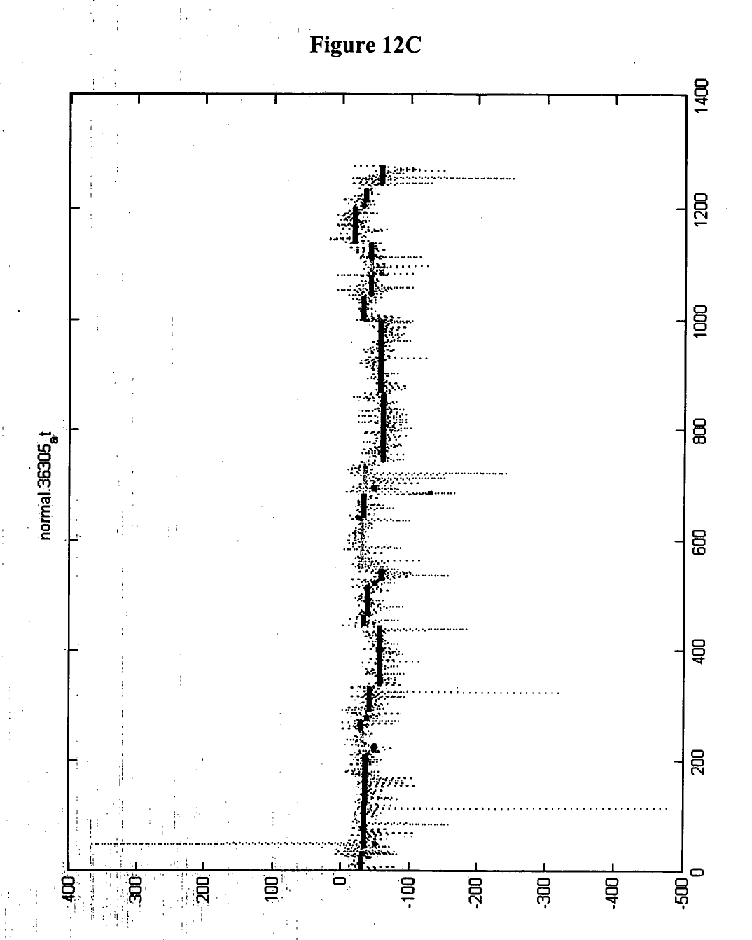

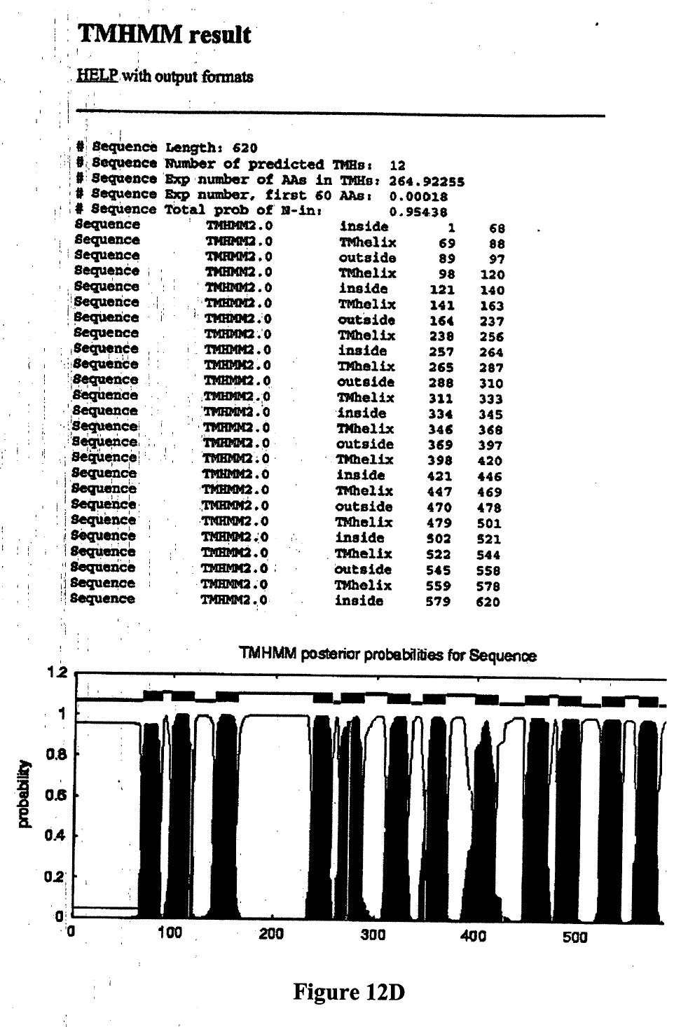

The expression of SLC6A3 in tumor and control tissue samples is shown in FIGS. 12A-12C. The TMHMM profile of a SLC6A3 protein is illustrated in FIG. 12D. The median expression level of SLC6A3 in kidney cancer samples is about 4.8-fold higher than that in the corresponding normal kidney tissue samples. Thus, SLC6A3 can be a diagnostic marker and drug target for kidney cancer.

SLC30A4 (solute carrier family 30 (zinc transporter), member 4) encodes the fourth and last identified member of a mammalian proteins family presumably involved in the cellular transport of zinc. Zinc is an essential trace element required by many living organisms. An adequate supply of zinc is particularly important in the neonatal period. Zinc is a significant component of breast milk, which is transported across the maternal epithelia during lactation. The mechanisms by which zinc becomes a constituent of breast milk have not been elucidated. The function of the zinc transporter ZnT4 in the transport of zinc into milk during lactation was previously demonstrated by studies of a mouse mutant, the ‘lethal milk’ mouse, where a mutation in the ZnT4 gene decreased the transport of zinc into milk.

The SLC30A4 gene is the human homologue of murine ZnT4 gene. There is strong homology between the mouse ZnT4 and human SLC30A4 coding sequences. The SLC30A4 gene is constitutively expressed in the human breast and may be one of the several members of the ZnT family involved in the transport of zinc into milk.

Kidneys also play a key role in zinc balance. The portion of Zn²⁺ that enters the glomerular filtrate is efficiently reabsorbed along the nephron through a mechanism yet to be identified. SLC30A4 gene expression was detected in adult rat kidney. The gene has not yet been associated with cancer.

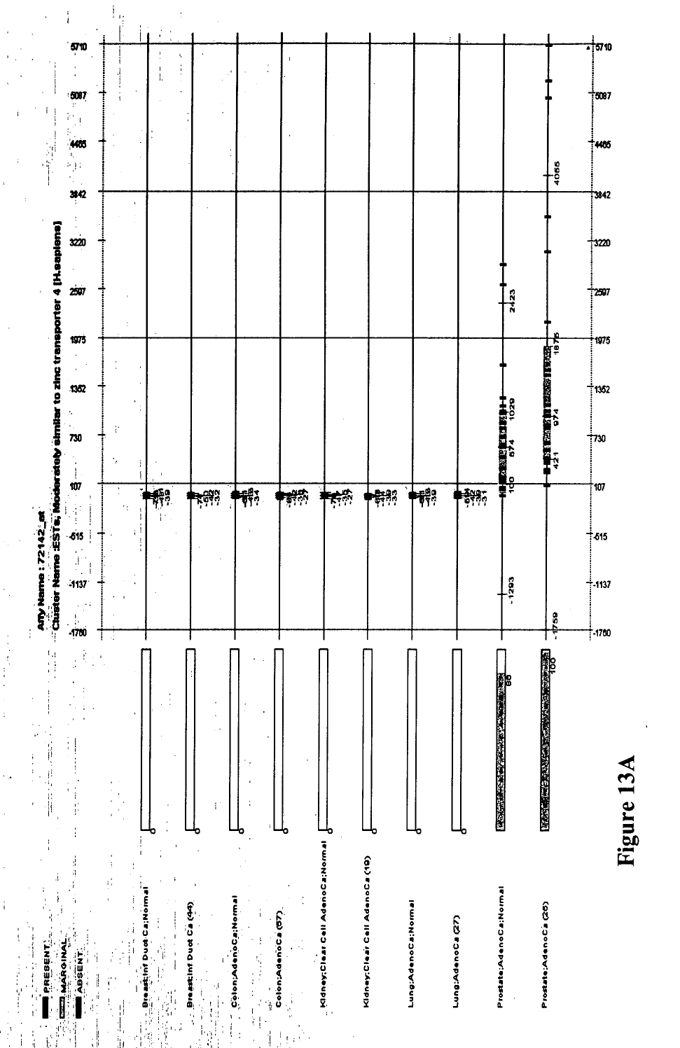

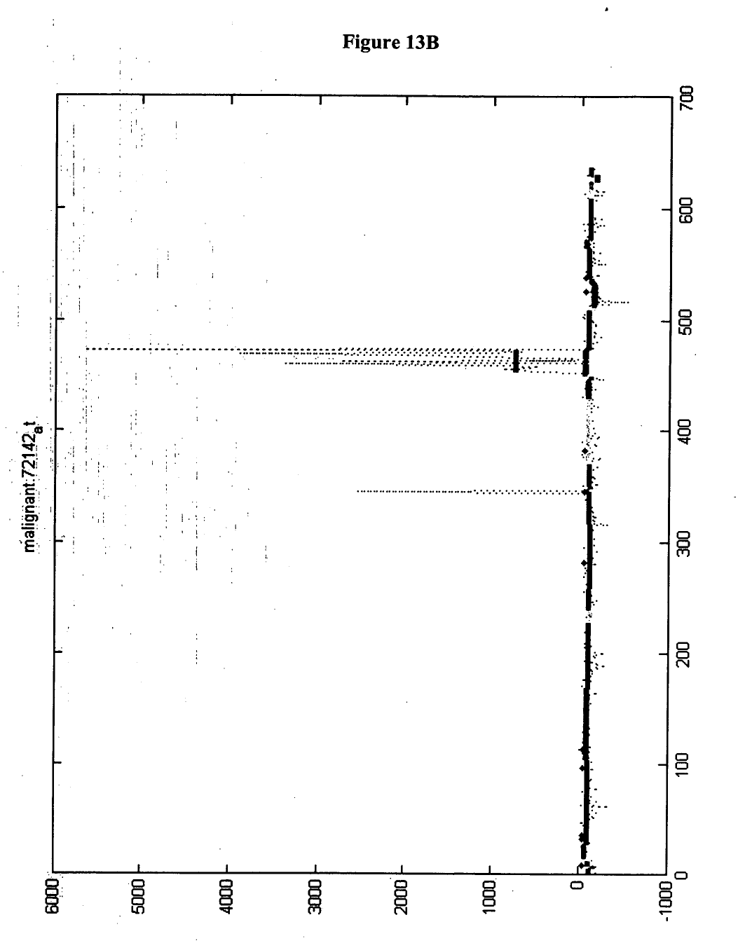

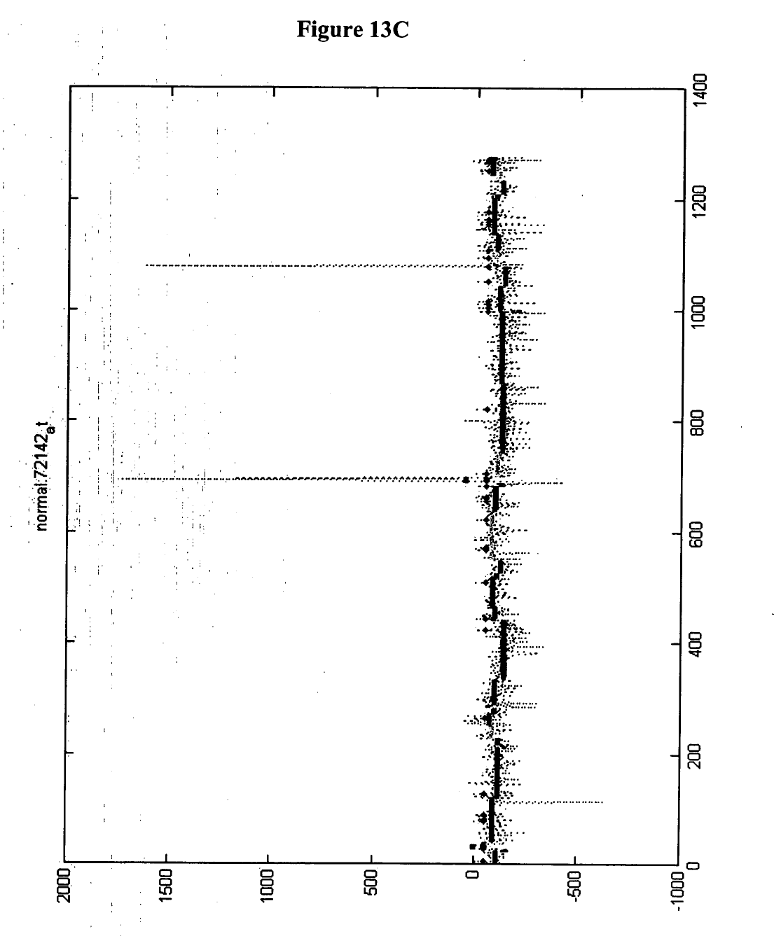

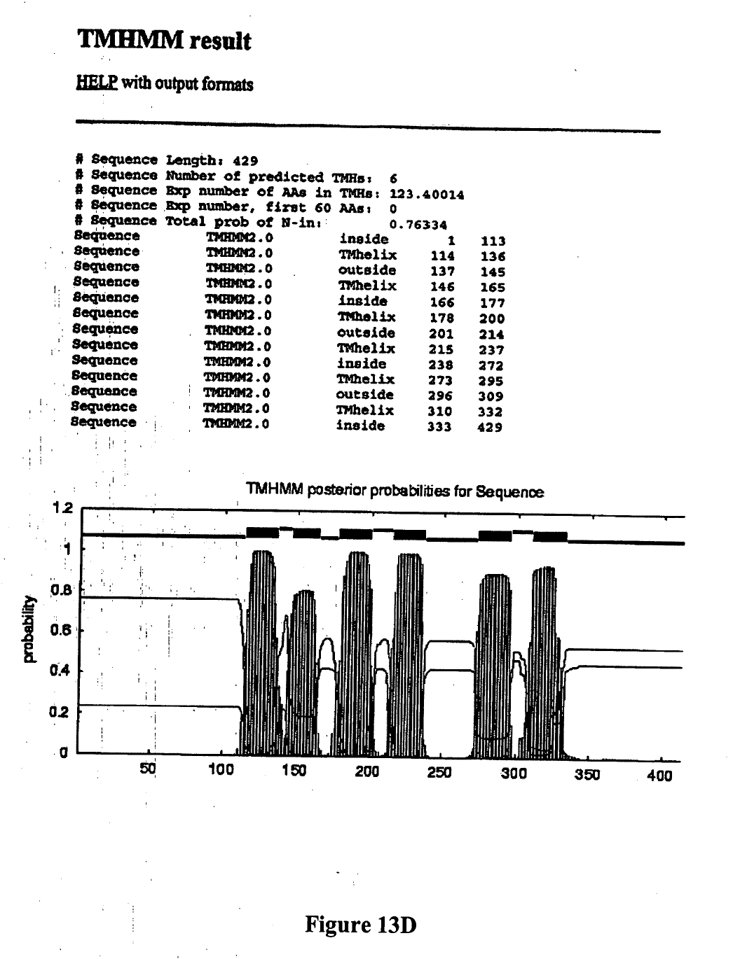

The expression of SLC30A4 in tumor and control tissue samples is shown in FIGS. 13A-13C. The TMHMM profile of a SLC30A4 protein is illustrated in FIG. 13D. SLC30A4 can be a diagnostic marker and drug target for prostate cancer.

TRG@ (T cell receptor gamma locus) encodes the gamma chain of a T cell receptor. T cell receptors recognize foreign antigens which have been processed as small peptides and bound to major histocompatibility complex (MHC) molecules at the surface of antigen presenting cells (APC). Each T cell receptor is a dimer consisting of one alpha and one beta chain or one delta and one gamma chain. In a single cell, the T cell receptor loci are rearranged and expressed in the order delta, gamma, beta, and alpha. If both delta and gamma rearrangements produce functional chains, the cell expresses delta and gamma. If not, the cell proceeds to rearrange the beta and alpha loci. The TRG@ region represents the germline organization of the T cell receptor gamma locus. The gamma locus includes V (variable), J (oining), and C (constant) segments. During T cell development, the gamma chain is synthesized by a recombination event at the DNA level joining a V segment with a J segment; the C segment is later joined by splicing at the RNA level. Recombination of many different V segments with several J segments provides a wide range of antigen recognition. Additional diversity is attained by junctional diversity, resulting from the random additional of nucleotides by terminal deoxynucleotidyltransferase. Several V segments of the gamma locus are known to be incapable of encoding a protein and are considered pseudogenes. Somatic rearrangement of the gamma locus has been observed in T cells derived from patients with T cell leukemia and ataxia telangiectasia. The polymorphism of the TCR-gamma complex may also be involved in the pathogenesis of colorectal cancer (Uthoff et al., Ann. Surg. Oncol., 9:88-93, 2002).

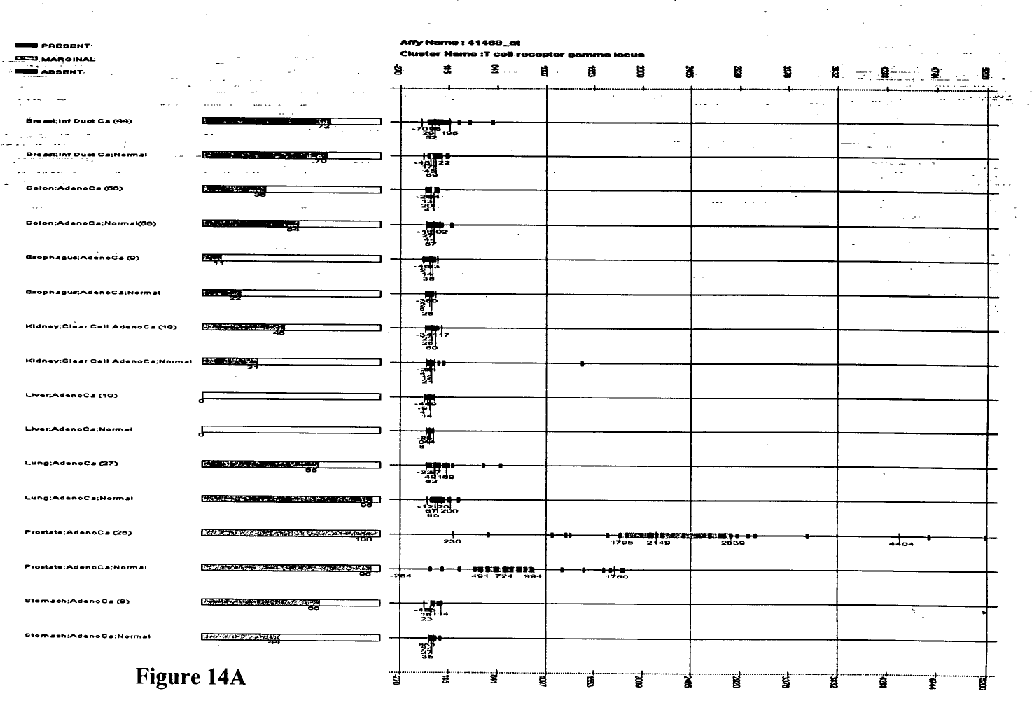

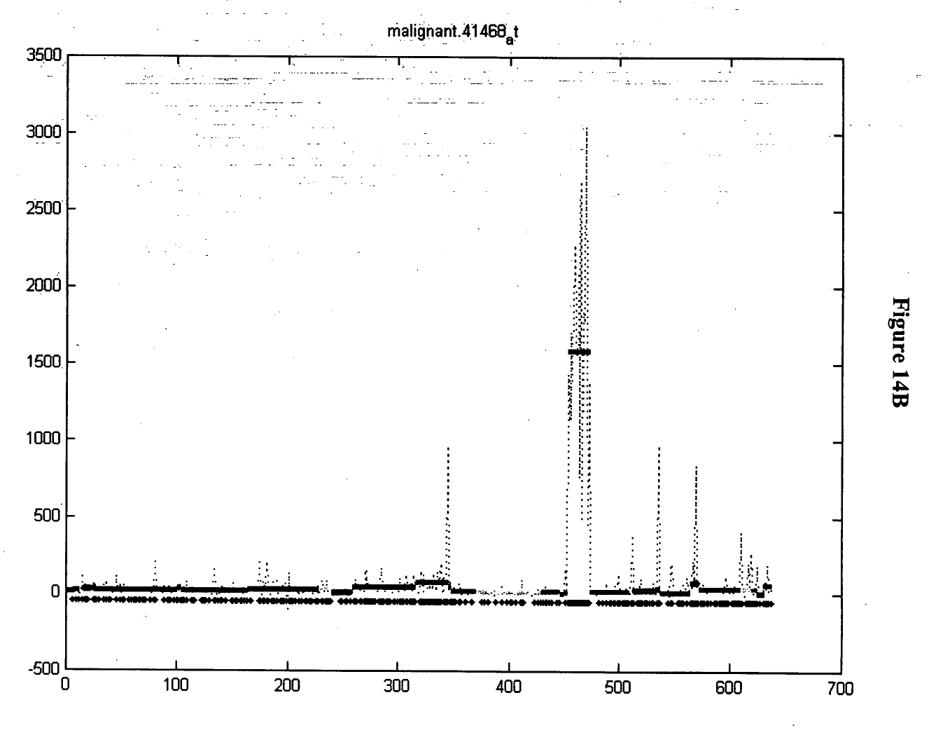

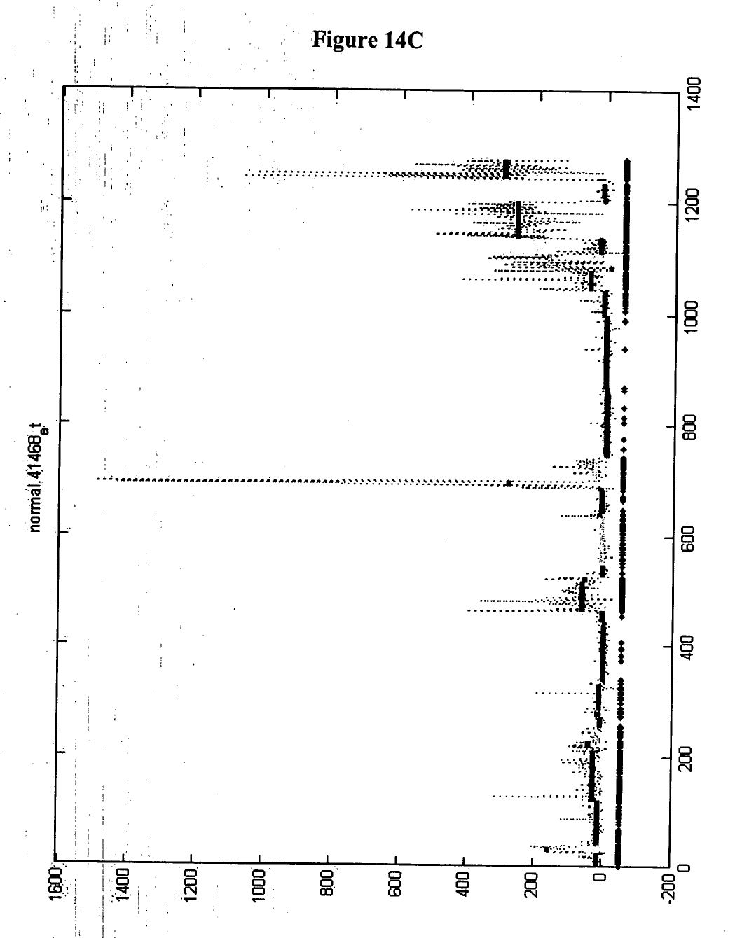

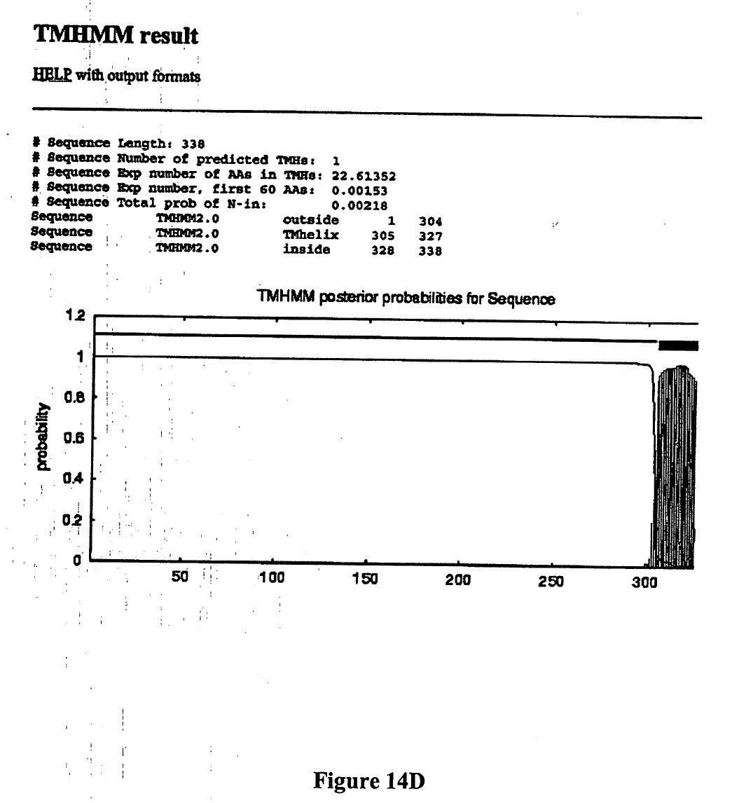

The expression of TRG@ in tumor and control tissue samples is shown in FIGS. 14A-14C. The TMHMM profile of a TRG@ protein is illustrated in FIG. 14D. The median expression level of TRG@ in prostate cancer samples is about 3-fold higher than that in the corresponding normal prostate tissue samples. Hence, TRG@ can be a diagnostic marker and drug target for prostate cancer.

TRPM4 (transient receptor potential cation channel, subfamily M, member 4) encodes a protein which belongs to the transient receptor potential (TRP) superfamily. The superfamily includes a group of subfamilies of channel-like proteins mediating a multitude of physiological signaling processes. The TRP-melastatin (TRPM) subfamily includes the putative tumor suppressor melastatin (MLSN) and is a poorly characterized group of TRP-related proteins. TRPM4 and MLSN each mediate Ca²⁺ entry when expressed in HEK293 cells.

Recently, a second form of TRPM4, TRPM4b, was cloned and characterized. TRPM4b encodes a cation channel of 25 pS unitary conductance that is directly activated by [Ca²⁺] with an apparent K(D) of approximately 400 nM. TRPM4b is activated following receptor-mediated Ca²⁺ mobilization, representing a regulatory mechanism that controls the magnitude of Ca²⁺ influx by modulating the membrane potential and, with it, the driving force for Ca²⁺ entry through other Ca²⁺-permeable pathways.

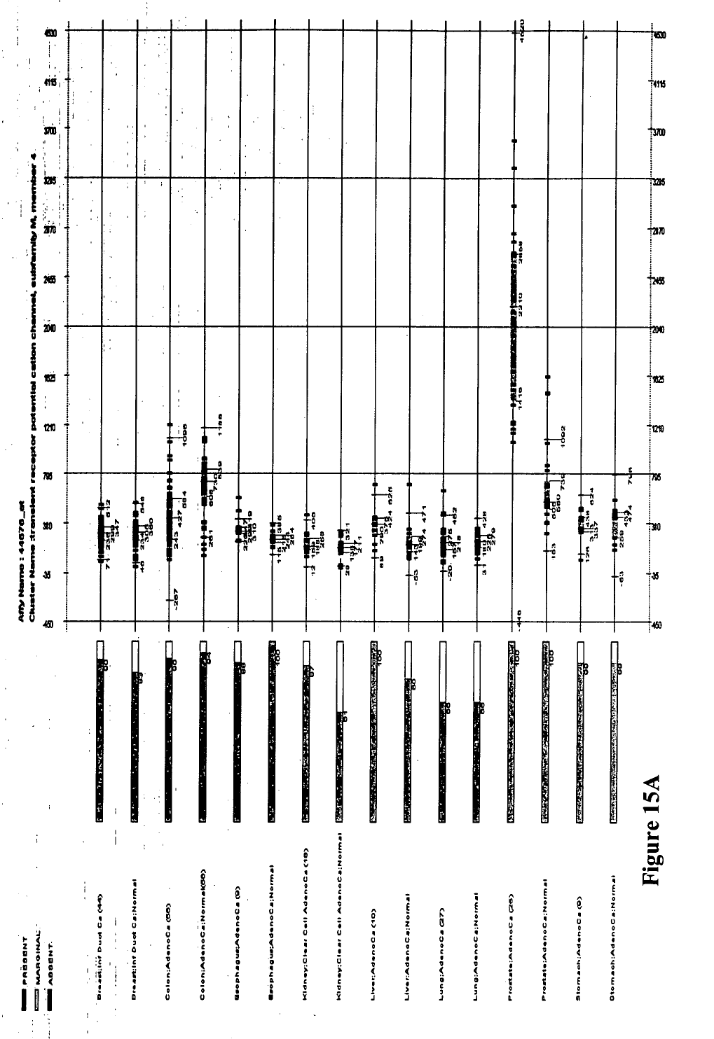

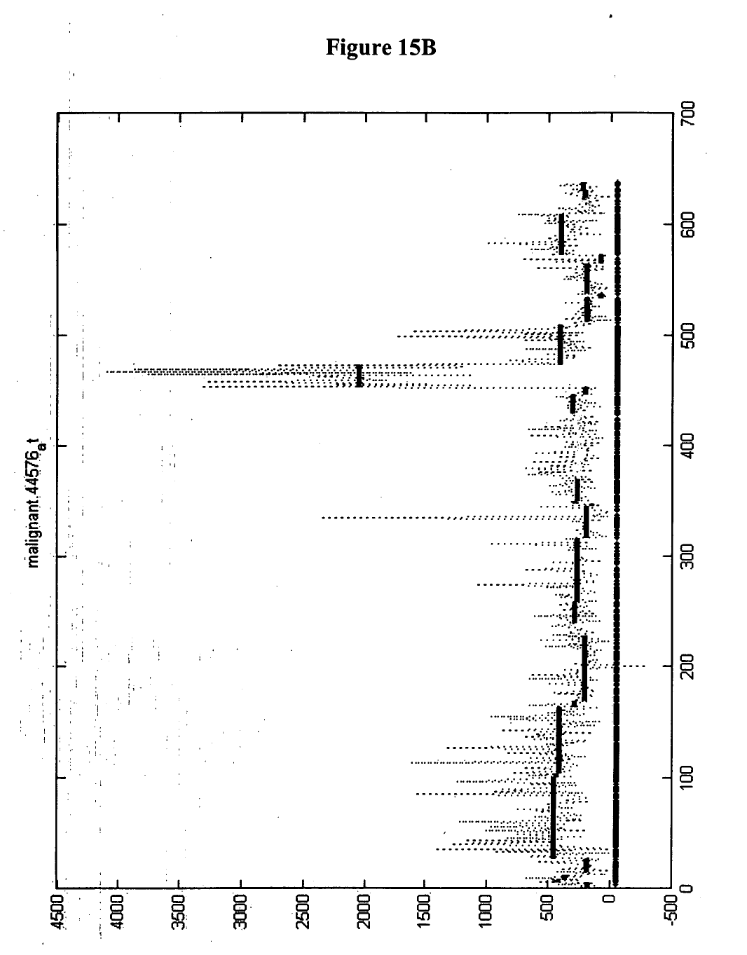

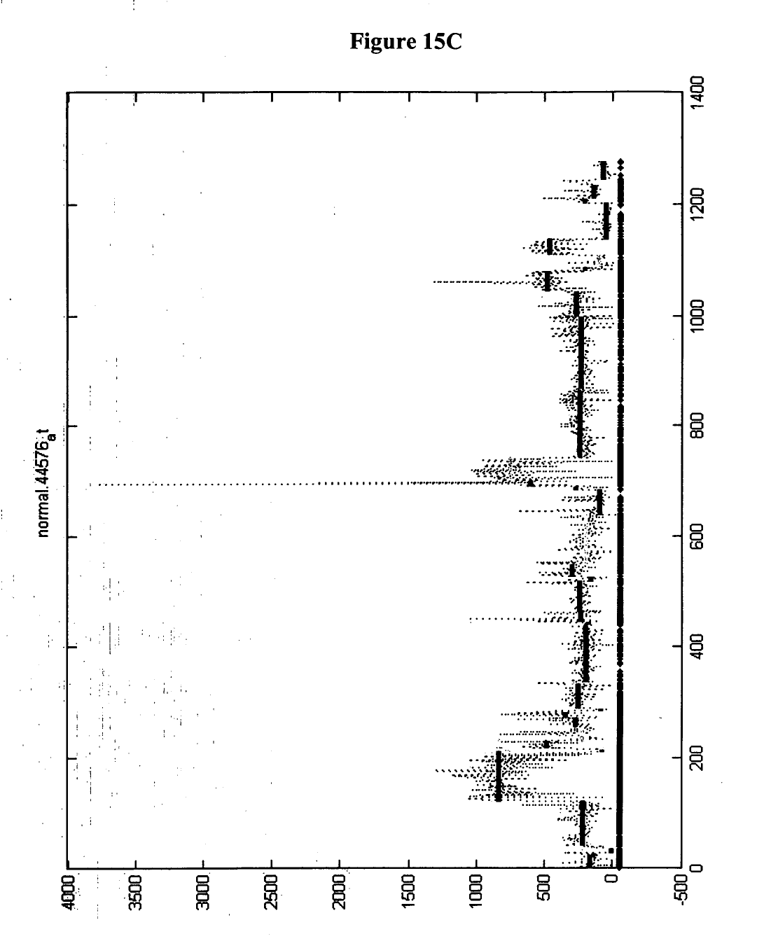

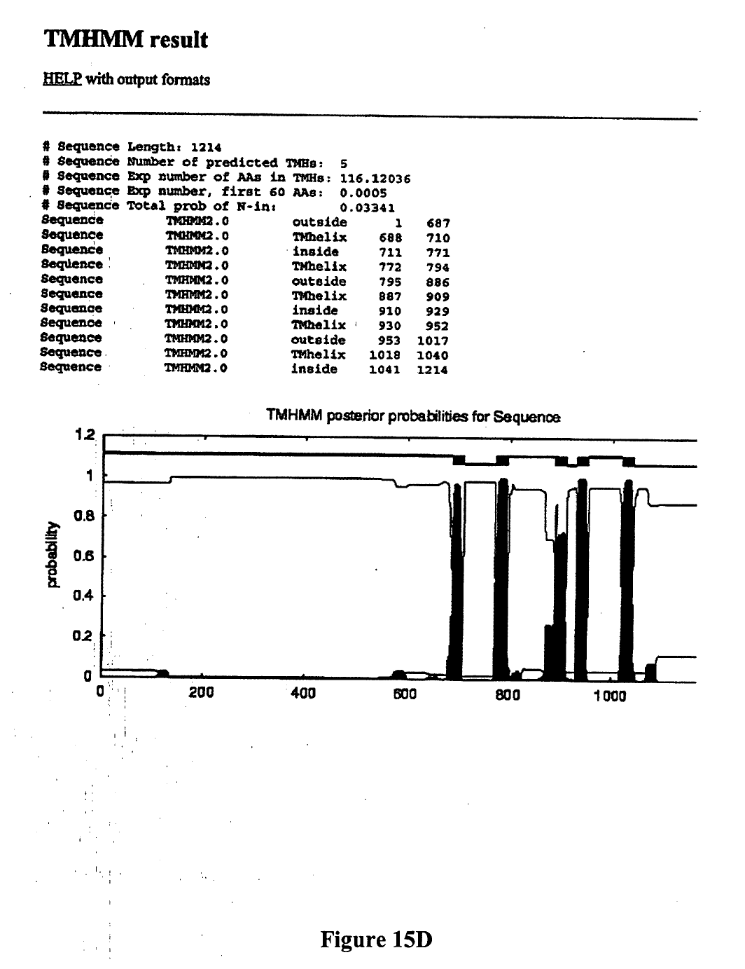

The expression of TRPM4 in tumor and control tissue samples is shown in FIGS. 15A-15C. The TMHMM profile of a TRPM4 protein is illustrated in FIG. 15D. The median expression level of TRPM4 in prostate cancer samples is about 4-fold higher than that in the corresponding normal prostate tissue samples. Therefore, TRPM4 can be a diagnostic marker and drug target for prostate cancer.

Table 1b summarizes each cancer and the CRTPGs that can be used for the diagnosis and treatment of that cancer.

TABLE 1b
CRTPGs for Diagnosis and Treatment of Cancers

| Cancer Type | Corresponding CRTPGs |
|---|---|
| prostate cancer | ABCC4, CACNA1D, OR51E2, TRG@, TRPM4, |
| | SLC30A4, and FOLH1 |
| kidney cancer | SLC30A4, CDH6, ENPP3, CST, GRP54, and HAVCR1 |
| breast cancer | FLJ11856, C20orf103, CACNA1D, and GPR54 |
| esophageal | FLJ11856, C20orf103, CACNA1D, and CST |
| cancer | |
| liver cancer | FLJ11856, CACNA1D, and CST |
| colon cancer | CACNA1D and FLJ11856 |
| lung cancer | FLJ11856, GPR54, C20orf103, CST, and ENPP3 |
| stomach cancer | CDH6 and FLJ11856 |

CRTPGs as Targets of Cancer Therapy and Markers for Cancers

The present invention provides methods of using CRTPGs or their expression products for detecting, monitoring, evaluating, or treating cancers. In one embodiment, cancer cells are targeted with anti-CRTP antibodies that are capable of inducing antibody-dependent cellular cytotoxicity (ADCC) or sensitizing the tumor cells to a cytotoxic factor such as TNFa. In one example, the anti-CRTP antibodies are conjugated to a toxic drug moiety, such as calicheamicin and esperamicin or a radiation source. In many embodiments, the anti-CRTP antibodies are monoclonal or humanized antibodies.

In another embodiment, a humoral immune response is induced against cancer cells in a patient of interest. A humoral immune response can be elicited by using a vaccine comprising a CRTP or a fragment thereof or an expression vector encoding the same.

In yet another embodiment, a modulator of the expression or protein activity of a CRTPG is administered to a patient in need thereof. Without being limited to any particular mechanism, the invention is based in part on the principle that modulation of the expression or protein activities of CRTPGs can ameliorate cancer when the expression levels or activities of these genes are returned to or substantially returned to normal (non-diseased) levels. The modulation of CRTPGs can be achieved at the protein level, for example, by inhibiting the activity of a CRTP with an antibody, an antagonist, or a dominant negative mutant. The modulation of CRTPGs can also be achieved at the transcriptional level, for example, by inhibiting the expression of a CRTPG with an antisense nucleic acid, an RNAi sequence, or an inhibitor to the CRTPG promoter or other regulatory sequences.

The present invention also features the use of CRTPGs or their expression products as cancer markers. In one embodiment, CRTPGs are used to detect, monitor or evaluate the presence, progression, prognosis, or clinical outcome of cancer. In another embodiment, CRTPGs are used to assess the efficacy of a treatment of cancer. In many embodiments, relative expression levels of CRTPGs are indicative of the severity of cancer.

The present invention further features methods of using CRTPGs to screen for anti-cancer compounds. In one embodiment, the compounds thus identified can convert an expression profile that indicates a poor prognosis to one that is predictive of a better prognosis.

The CRTPG products can be isolated from respective body fluids, tissues or cells. The CRTPG products can also be prepared by using recombinant DNA or chemical synthesis techniques, as appreciated by those skilled in the art.

Isolated Polynucleotides

Many polynucleotides employed in the present invention comprise sequences which are substantially identical or complementary to the CRTPNs listed in Table 1a. A sequence is substantially complementary to another sequence if the two sequences can hybridize to each other under highly stringent conditions.

Examples of conditions of different hybridization stringency are listed in Table 2. Highly stringent conditions are those that are at least as stringent as conditions A-F; stringent conditions are at least as stringent as conditions G-L; and reduced stringency conditions are at least as stringent as conditions M-R. As used in Table 2, hybridization is carried out under a given hybridization condition for about 2 hours, followed by two 15-minute washes under the corresponding washing condition(s).

TABLE 2
Stringency Conditions

| Stringency | Polynucleotide | Hybrid | Hybridization | Wash Temp. |
|---|---|---|---|---|
| Condition | Hybrid | Length (bp)¹ | Temperature and Buffer^{H} | and Buffer^{H} |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | TB*; 1xSSC | TB*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | TD*; 1xSSC | TD*; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | TF*; 1xSSC | TF*; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | TH*; 4xSSC | TH*; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | TJ*; 4xSSC | TJ*; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | <50 | TL*; 2xSSC | TL*; 2xSSC |
| M | DNA:DNA | >50 | 50° C.; 4xSSC -or- | 50° C.; 2xSSC |
| | | | 40° C.; 6xSSC, 50% formamide | |
| N | DNA:DNA | <50 | TN*; 6xSSC | TN*; 6xSSC |
| O | DNA:RNA | >50 | 55° C.; 4xSSC -or- | 55° C.; 2xSSC |
| | | | 42° C.; 6xSSC, 50% formamide | |
| P | DNA:RNA | <50 | TP*; 6xSSC | TP*; 6xSSC |
| Q | RNA:RNA | >50 | 60° C.; 4xSSC -or- | 60° C.; 2xSSC |
| | | | 45° C.; 6xSSC, 50% formamide | |
| R | RNA:RNA | <50 | TR*; 4xSSC | TR*; 4xSSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides | | | | |
| # and identifying the region or regions of optimal sequence complementarity. | | | | |
| ^{H}SSPE (1xSSPE is 0.15M NaCl, 10 mM NaH2PO4, and 1.25 mM EDTA, pH 7.4) can be substituted for SSC (1xSSC is 0.15M NaCl and 15 mM sodium citrate) in the hybridization and wash buffers. | | | | |
| TB* - TR*: The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.) = 2(# of A + T bases) + 4(# of G + C bases). For hybrids between 18 and 49 base pairs in | | | | |
| # length, Tm(° C.) = 81.5 + 16.6(log₁₀Na⁺) + 0.41(% G + C) − (600/N), where N is the number of bases in the hybrid, and Na⁺ is the molar concentration of sodium ions in the hybridization buffer (Na⁺ for 1xSSC = 0.165M). | | | | |

Many polynucleotides of the inventon can be used as probes or primers. A probe/primer typically comprises a substantially purified polynucleotide. In many embodiments, a probe/primer includes at least about 15, 25, 50, 75, 100, 125, 150, 175, 200, 225, 250, 275, 300, 325, 350, 400 or more consecutive nucleotides, and can hybridize under stringent conditions to of a CRTPG or CRTPN, or the complement thereof. As used herein, a polynucleotide can gybridize to a gene if the polynucleotide can hybridize ti an RNA transcript, or the complement thereof, of the gene. In many embodimetns, probes for a given gene do not hybridize to other genes under stringent conditions.

In one embodiment, the probes employed in the present invention comprise label groups. Suitable label groups include, but are not limited to, radioisotopes, fluorescent compounds, enzymes, or enzyme co-factors. The probes can also be labeled indirectly. The probes of the present invention can be used as part of a diagnostic kit for identifying cells or tissues in which a CRTPG is over- or under-expressed. The probes can also be used to detect the number of genomic copies of a CRTPG in a patient of interest.

The invention further features polynucleotide variants. The polynucleotide variants of the present invention include polynucleotide molecules differing from the polynucleotide sequences of the CRTPGs listed in Table 1a due to degeneracy of the genetic code but encoding the same proteins encoded by CRTPGs shown in Table 1a. The polynucleotide variants of the present invention also include CRTPG homologs of other species. In addition, the polynucleotide variants of the present invention include polynucleotide molecules which are structurally different from the CRTPNs (e.g., having a slight altered sequence), but which have substantially the same properties as the CRTPNs (e.g., encoding similar amino acid sequences that are changed only at non-essential amino acid residues). Such polynucleotides may differ from the original polynucleotide by one or more substitutions, additions, and/or deletions. For instance, a variant of a polynucleotide can have 1, 2, 5, 10, 15, 20, 25 or more nucleotide substitutions, additions or deletions, but still encodes a polypeptide having substantially the same function as the polypeptide encoded by the original polynucleotide. In many cases, the modification(s) is in-frame, i.e., the modified polynucleotide can be transcribed and translated to the original or intended stop codon. If the original polynucleotide encodes a polypeptide with a biological activity, the polypeptide encoded by a variant of the original polynucleotide substantially retains such activity. In one embodiment, the biological activity of a variant is reduced or enhanced by no more than 50%, 40%, 30%, 20%, 10% or 5% relative to the original activity. Examples of polynucleotide variants include allelic variants.

Furthermore, the polynucleotide variants of the present invention include polynucleotide molecules that are capable of hybridizing under stringent or highly stringent conditions to a CRTPN or CRTPG, or the complement thereof. These polynucleotide molecules can include at least 15, 20, 25, 30, 50, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000 or more nucleotides.

In addition to the nucleotide sequences of the CRTPGs listed in Table 1a, it will be appreciated by those skilled in the art that DNA sequence polymorphisms leading to changes in the amino acid sequences of the proteins encoded by the CRTPGs listed in Table 1a may exist within a population (e.g., the human population). These polymorphic DNA sequences are contemplated by the present invention. Such genetic polymorphism in the CRTPGs listed in Table 1a may exist among individuals within a population due to natural allelic variation. In addition, it will be appreciated that DNA polymorphisms that affect RNA expression levels can also exist and may affect the overall expression level of that gene (e.g., by affecting regulation or degradation).

Polynucleotide molecules corresponding to natural allelic variants and homologs of the CRTPGs can be isolated based on their homology to the CRTPGs listed in Table 1a using standard recombinant DNA technology. Polynucleotide molecules corresponding to natural allelic variants and homologs of the CRTPGs of the invention can also be isolated by mapping to the same chromosome or locus as the CRTPGs of the invention.

A polynucleotide may be further modified to increase stability in vivo. Possible modifications include, but are not limited to, the addition of flanking sequences at the 5′ and/or 3′ ends; the use of phosphorothioate or 2-o-methyl rather than phosphodiester linkages in the backbone; and the inclusion of nontraditional bases such as inosine, queosine and wybutosine, as well as acetyl- methyl-, thio- and other modified forms of adenine, cytidine, guanine, thymine and uridine.

Another aspect of the invention pertains to isolated polynucleotide molecules that are antisense to the CRTPGs of the invention. An “antisense” polynucleotide comprises a nucleotide sequence which is complementary to a “sense” polynucleotide, e.g., complementary to the coding strand of a double-stranded cDNA molecule or to an mRNA sequence. An antisense polynucleotide can be complementary to the entire coding strand of a CRTPG, or a portion thereof. An antisense polynucleotide molecule can also be complementary to a noncoding region of the coding strand of a CRTPG or CRTPN.

In many embodiments, an antisense polynucleotide of the present invention includes at least about 10, 15, 20, 25, 30, 35, 40, 45, 50 or more nucleotides. An antisense polynucleotide can be designed according to the rules of Watson and Crick base pairing. In one embodiment, an antisense polynucleotide is chemically synthesized using naturally occurring nucleotides or variously modified nucleotides designed to increase the biological stability of the molecules or to increase the physical stability of the duplex formed between the antisense and sense polynucleotides. Examples of modified nucleotides which can be used to generate an antisense polynucleotide include 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladen4exine, unacil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, (acp3)w, and 2,6-diaminopurine. In another embodiment, an antisense polynucleotide of the present invention is produced biologically by using an expression vector into which the target sequence is subcloned in an antisense orientation.

In many cases, the antisense polynucleotide molecules of the invention are administered to a subject or generated in situ such that they hybridize or bind to cellular mRNA and/or genomic DNA encoding a CRTP, thereby inhibiting the expression of the corresponding CRTPG. An exemplary route of administration of antisense polynucleotide molecules is direct injection at a tissue site (e.g., intestine). In one embodiment, antisense polynucleotide molecules are modified to target selected cells and then administered systemically. For systemic administration, antisense molecules can be modified such that they specifically bind to receptors or antigens expressed on a selected cell surface, e.g., by linking the antisense polynucleotide molecules to peptides or antibodies which bind to cell surface receptors or antigens. The antisense polynucleotide molecules can also be delivered to cells using the vectors described herein. To achieve sufficient intracellular concentrations of the antisense molecules, vector constructs in which the antisense polynucleotide molecule is placed under the control of a strong promoter, such as pol II or pol III promoter, may be employed.

In yet another embodiment, an antisense polynucleotide molecule of the invention is an α-anomeric polynucleotide molecule. An α-anomeric polynucleotide molecule forms specific double-stranded hybrids with complementary RNA in which, contrary to the usual β-units, the strands run parallel to each other (Gaultier et al., Polynucleotides. Res., 15:6625-6641, 1987). The antisense polynucleotide molecule can also comprise a 2′-o-methylribonucleotide or a chimeric RNA-DNA analogue.

In still another embodiment, an antisense polynucleotide of the invention is a ribozyme. Ribozymes are catalytic RNA molecules with ribonuclease activity which are capable of cleaving a single-stranded polynucleotide, such as an mRNA, to which they have a complementary region. Thus, ribozymes (e.g., hammerhead ribozymes) can be used to catalytically cleave mRNA transcripts of the CRTPGs to thereby inhibit translation of said mRNA. A ribozyme having specificity for a CRTPN can be designed based upon the nucleotide sequence of a gene of the invention, disclosed herein. For example, a derivative of a Tetrahymena L-19 IVS RNA can be constructed in which the nucleotide sequence of the active site is complementary to the nucleotide sequence to be cleaved in a CRTPG protein-encoding mRNA. Alternatively, mRNA transcribed from a gene of the invention can be used to select a catalytic RNA having a specific ribonuclease activity from a pool of RNA molecules. Alternatively, expression of a CRTPG of the invention can be inhibited by targeting the regulatory region of these genes (e.g., the promoter and/or enhancers) with complementary nucleotide sequences that will form triple helical structures with the target sequence to prevent transcription of the gene in target cells.

Expression of the CRTPGs of the invention can also be inhibited by using RNA interference (“RNAᵢ”). Sequences capable of inhibiting gene expression by RNA interference can have any desired length. For instance, the sequence can have at least 15, 20, 25, or more consecutive nucleotides. The sequence can be dsRNA or any other type of polynucleotide, provided that the sequence can form a functional silencing complex to degrade the target mRNA transcript.

In one embodiment, the sequence comprises or consists of a short interfering RNA (siRNA). The siRNA can be, without limitation, dsRNA having 19-25 nucleotides. siRNAs can be produced endogenously by degradation of longer dsRNA molecules by an RNase III-related nuclease called Dicer. siRNAs can also be introduced into a cell exogenously or by transcription of an expression construct. Once formed, the siRNAs assemble with protein components into endoribonuclease-containing complexes known as RNA-induced silencing complexes (RISCs). An ATP-generated unwinding of the siRNA activates the RISCs, which in turn target the complementary mRNA transcript by Watson-Crick base-pairing, thereby cleaving and destroying the mRNA. Cleavage of the mRNA may take place near the middle of the region bound by the siRNA strand. This sequence-specific mRNA degradation results in gene silencing.

At least two ways can be employed to achieve siRNA-mediated gene silencing. First, siRNAs can be synthesized in vitro and introduced into cells to transiently suppress gene expression. Synthetic siRNA provides an easy and efficient way to achieve RNAi. siRNA are duplexes of short mixed oligonucleotides which can include, for example, 19 nucleotides with symmetric dinucleotide 3′ overhangs. Using synthetic 21 bp siRNA duplexes (e.g., 19 RNA bases followed by a UU or dTdT 3′ overhang), sequence-specific gene silencing can be achieved in mammalian cells. These siRNAs can specifically suppress targeted gene translation in mammalian cells without activation of DNA-dependent protein kinase (PKR) by longer dsRNA, which may result in non-specific repression of translation of many proteins.

Second, siRNAs can be expressed in vivo from vectors. This approach can be used to stably express siRNAs in cells or transgenic animals. In one embodiment, siRNA expression vectors are engineered to drive siRNA transcription from polymerase III (pol III) transcription units. Pol III transcription units are suitable for hairpin siRNA expression, since they deploy a short AT rich transcription termination site that leads to the addition of 2 bp overhangs (e.g., UU) to hairpin siRNAs—a feature that is helpful for siRNA function. The Pol III expression vectors can also be used to create transgenic mice that express siRNA.

In another embodiment, siRNAs can be expressed in a tissue-specific manner. Under this approach, long double-stranded RNAs (dsRNAs) are first expressed from a promoter (such as CMV (pol II)) in the nuclei of selected cell lines or transgenic mice. The long dsRNAs are processed into siRNAs in the nuclei (e.g., by Dicer). The siRNAs exit from the nuclei and mediate gene-specific silencing. A similar approach can be used in conjunction with tissue-specific promoters to create tissue-specific knockdown mice.

Any 3′ dinucleotide overhang, such as UU, can be used for siRNA design. In some cases, G residues in the overhang are avoided because of the potential for the siRNA to be cleaved by RNase at single-stranded G residues.

With regard to the siRNA sequence itself, it has been found that siRNAs with 30-50% GC content can be more active than those with a higher G/C content in certain cases. Moreover, since a 4-6 nucleotide poly(T) tract may act as a termination signal for RNA pol III, stretches of >4 Ts or As in the target sequence may be avoided in certain cases when designing sequences to be expressed from an RNA pol III promoter. In addition, some regions of mRNA may be either highly structured or bound by regulatory proteins. Thus, it may be helpful to select siRNA target sites at different positions along the length of the gene sequence. Finally, the potential target sites can be compared to the appropriate genome database (human, mouse, rat, etc.). Any target sequences with more than 16-17 contiguous base pairs of homology to other coding sequences may be eliminated from consideration in certain cases.

In one embodiment, siRNA is designed to have two inverted repeats separated by a short spacer sequence. In some examples, the siRNA end with a string of Ts that serve as a transcription termination site. This design produces an RNA transcript that is predicted to fold into a short hairpin siRNA. The selection of siRNA target sequence, the length of the inverted repeats that encode the stem of a putative hairpin, the order of the inverted repeats, the length and composition of the spacer sequence that encodes the loop of the hairpin, and the presence or absence of 5′-overhangs, can vary to achieve desirable results.

The siRNA targets can be selected by scanning an mRNA sequence for AA dinucleotides and recording the 19 nucleotides immediately downstream of the AA. Other methods can also been used to select the siRNA targets. In one example, the selection of the siRNA target sequence is purely empirically determined (see e.g., Sui et al, Proc. Natl. Acad. Sci. USA 99: 5515-5520, 2002), as long as the target sequence starts with GG and does not share significant sequence homology with other genes as analyzed by BLAST search. In another example, a more elaborate method is employed to select the siRNA target sequences. This procedure exploits an observation that any accessible site in endogenous mRNA can be targeted for degradation by synthetic oligodeoxyribonucleotide/RNase H method (Lee et al, Nature Biotechnology 20:500-505, 2002).

In another embodiment, the hairpin siRNA expression cassette is constructed to contain the sense strand of the target, followed by a short spacer, the antisense strand of the target, and 5-6 Ts as transcription terminator. The order of the sense and antisense strands within the siRNA expression constructs can be altered without affecting the gene silencing activities of the hairpin siRNA. In certain instances, the reversal of the order may cause partial reduction in gene silencing activities.

The length of nucleotide sequence being used as the stem of siRNA expression cassette can range, for instance, from 19 to 29. The loop size can range from 3 to 23 nucleotides. Other lengths and/or loop sizes can also be used.

In yet another embodiment, a 5′ overhang in the hairpin siRNA construct can be used, provided that the hairpin siRNA is functional in gene silencing. In one example, the 5′ overhang includes about 6 nucleotide residues.

In still yet another embodiment, the target sequences for RNAi are 21-mer or 20-mer sequence fragments selected from CRTPG coding sequences, such as SEQ ID NOS:1-15. The target sequences can be selected from either ORF regions or non-ORF regions. The 5′ end of each target sequence has dinucleotide “NA,” where “N” can be any base and “A” represents adenine. The remaining 19-mer or 18-mer sequence has a GC content of between 30% and 65%. In many examples, the remaining 19-mer or 18-mer sequence does not include any four consecutive A or T (i.e., AAAA or TTTT), three consecutive G or C (i.e., GGG or CCC), or seven “GC” in a row. Examples of the target sequences prepared using the above-described criteria (“Relaxed Criteria”) are illustrated in Table 3. Each target sequence in Table 3 has SEQ ID NO:3n+1, and the corresponding siRNA sense and antisense strands have SEQ ID NO:3n+2 and SEQ ID NO:3n+3, respectively, where n is a positive integer. For each CRTPG coding sequence (SEQ ID NOS:1-15), multiple target sequences can be selected.

Additional criteria can be used for RNAi target sequence design. In one example, the GC content of the remaining 19-mer or 18-mer sequence is limited to between 35% and 55%, and any 19-mer or 18-mer sequence having three consecutive A or T (i.e., AAA or TTT) or a palindrome sequence with 5 or more bases is excluded. In addition, the 19-mer or 18-mer sequence can be selected to have low sequence homology to other human genes. In one embodiment, potential target sequences are searched by BLASTN against NCBI's human UniGene cluster sequence database. The human UniGene database contains non-redundant sets of gene-oriented clusters. Each UniGene cluster includes sequences that represent a unique gene. 19-mer/18-mer sequences producing no hit to other human genes under the BLASTN search can be selected. During the search, the e-value may be set at a stringent value (such as “1”). Furthermore, the target sequence can be selected from the ORF region, and is at least 75-bp from the start and stop codons. Examples of the target sequences (SEQ ID NO:3n+1) prepared using these criteria (“Stringent Criteria”) are demonstrated in Table 3. siRNA sense and antisense sequences (SEQ ID NO:3n+2 and SEQ ID NO:3n+3, respectively) are also provided.

TABLE 3
RNAi Target Sequences and siRNA Sequences for CRTPGs

| | | Relaxed Criteria: | Stringent Criteria: |
|---|---|---|---|
| | | Target Seq.: SEQ ID NO: 3n + 1 | Target Seq.: SEQ ID NO: 3n + 1 |
| | cDNA | siRNA Sense Seq.: SEQ ID NO: 3n + 2 | siRNA Sense Seq.: SEQ ID NO: 3n + 2 |
| Gene Name | (SEQ ID) | siRNA Antisense Seq.: SEQ ID NO: 3n + 3 | siRNA Antisense Seq.: SEQ ID NO: 3n + 3 |
| ABCC4 | 1 | SEQ ID NOs: 31-1,566 | SEQ ID NOs: 1,567-1,935 |
| C20orf103 | 2 | SEQ ID NOs: 1,936-2,481 | SEQ ID NOs: 2,482-2,535 |
| CACNA1D | 3 | SEQ ID NOs: 2,536-4,974 | SEQ ID NOs: 4,974-5,436 |
| CDH6 | 4 | SEQ ID NOs: 5,437-7,128 | SEQ ID NOs: 7,129-7,356 |
| CST | 5 | SEQ ID NOs: 7,357-7,668 | SEQ ID NOs: 7,669-7,677 |
| ENPP3 | 6 | SEQ ID NOs: 7,678-8,799 | SEQ ID NOs: 8,800-9,039 |
| FLJ11856 | 7 | SEQ ID NOs: 9,040-9,255 | SEQ ID NOs: 9,256-9,264 |
| FOLH1 | 8 | SEQ ID NOs: 9,265-10,218 | SEQ ID NOs: 10,219-10,254 |
| GPR54 | 9 | SEQ ID NOs: 10,255-10,353 | SEQ ID NOs: 10,354-10,365 |
| HAVCR1 | 10 | SEQ ID NOs: 10,366-11,046 | SEQ ID NOs: 11,047-11,145 |
| OR51E2 | 11 | SEQ ID NOs: 11,146-11,955 | |
| SLC6A3 | 12 | SEQ ID NOs: 11,956-12,807 | SEQ ID NOs: 12,808-12,831 |
| SLC30A4 | 13 | SEQ ID NOs: 12,832-13,476 | SEQ ID NOs: 13,477-13,575 |
| TRG@ | 14 | SEQ ID NOs: 13,576-14,199 | SEQ ID NOs: 14,200-14,217 |
| TRPM4 | 15 | SEQ ID NOs: 14,218-14,853 | SEQ ID NOs: 14,854-14,937 |

The effectiveness of the siRNA sequences can be evaluated using various methods known in the art. For instance, a siRNA sequence of the present invention can be introduced into a cell that expresses a CRTPG. The polypeptide or mRNA level of the CRTPG in the cell can be detected. A substantial change in the expression level of the CRTPG before and after the introduction of the siRNA sequence is indicative of the effectiveness of the siRNA sequence in suppressing the expression of the CRTPG. In one example, the expression levels of other genes are also monitored before and after the introduction of the siRNA sequence. A siRNA sequence which has inhibitory effect on the CRTPG expression but does not significantly affect the expression of other genes can be selected. In another example, multiple siRNA or other RNAi sequences can be introduced into the same target cell. These siRNA or RNAi sequences specifically inhibit the CRTPG gene expression but not the expression of other genes. In yet another example, siRNA or other RNAi sequences that inhibit the expression of both the CRTPG gene and other gene or genes can be used.

In yet another embodiment, the polynucleotide molecules of the present invention can be modified at the base moiety, sugar moiety or phosphate backbone to improve, e.g., the stability, hybridization, or solubility of the molecule. For example, the deoxyribose phosphate backbone of the polynucleotide molecules can be modified to generate peptide polynucleotides. As used herein, the terms “peptide polynucleotides” or “PNAs” refer to polynucleotide mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols.

PNAs can be used in therapeutic and diagnostic applications. For example, PNAs can be used as antisense or anti-gene agents for sequence-specific modulation of CRTPG expression by inducing transcription or translation arrest or inhibiting replication. PNAs of the polynucleotide molecules of the invention (e.g., set forth in Table 1a or homologs thereof) can also be used in the analysis of single base pair mutations in a gene, (e.g., by PNA-directed PCR clamping), as artificial restriction enzymes when used in combination with other enzymes (e.g., S1 nucleases) or as probes or primers for DNA sequencing or hybridization.

In another embodiment, PNAs can be modified to enhance their stability or cellular uptake by attaching lipophilic or other helper groups to PNA, by the formation of PNA-DNA chimeras, or by the use of liposomes or other techniques of drug delivery known in the art. For example, PNA-DNA chimeras of the polynucleotide molecules of the invention can be generated. Such chimeras allow DNA recognition enzymes, (e.g., RNase H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation. PNA-DNA chimeras can be synthesized by a variety of means. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a spacer between the PNA and the 5′ end of DNA. PNA monomers are then coupled in a stepwise manner to produce a chimeric molecule with a 5′ PNA segment and a 3′ DNA segment. Alternatively, chimeric molecules can be synthesized with a 5′ DNA segment and a 3′ PNA segment.

In other embodiments, a polynucleotide of the present invention can include other appended groups such as peptides (e.g., for targeting host cell receptors in vivo), or agents facilitating transport across the cell membrane or the blood-kidney barrier (see, e.g., PCT Publication No. W089/10134). In addition, a polynucleotide of the present invention can be modified with hybridization-triggered cleavage agents or intercalating agents. To this end, the polynucleotide may be conjugated to another molecule (e.g., a peptide, hybridization triggered cross-linking agent, transport agent, or hybridization-triggered cleavage agent). Finally, a polynucleotide of the present invention can be detectably labeled such that the label can be either directly detected (e.g., a radioactive or fluorescent label) or by the addition of another reagent (e.g., a substrate for an enzymatic label).

Isolated Polypeptides

Several aspects of the invention pertain to isolated CRTPs and biologically active portions thereof, as well as polypeptide fragments suitable for use as immunogens to raise anti-CRTP antibodies. A “biologically active portion” of a CRTP includes a fragment of a CRTP and exhibits at least one activity of the CRTP. The CRTPs or fragments thereof can be used as targets for developing agents that modulate CRTP-mediated activities.

In one embodiment, native CRTPs are isolated from cells or tissues by an appropriate purification scheme using standard protein purification techniques. In many embodiments, however, no purification will be necessary.

The invention provides CRTPs encoded by the CRTPGs set forth in Table 1a, or homologs thereof. In one embodiment, a CRTP homolog is substantially homologous to a CRTP encoded by a CRTPG selected from Table 1a, and retains a functional activity of the CRTP, yet differs in amino acid sequence due to natural allelic variation or mutagenesis. The CRTP homolog may include an amino acid sequence which has at least about 60%, 65%, 70%, 75%, 80%, 85%, 90%, 95%, 98% or more sequence identity or similarity to an amino acid sequence encoded by a CRTPG selected from Table 1a.

Sequence homology between two amino acid sequences or nucleotide sequences can be determined using a mathematical algorithm. In one embodiment, the percent identity between two amino acid sequences is determined by using the Needleman and Wunsch (J. Mol. Biol., 48:444-453, 1970) algorithm with either a Blossom 62 or PAM250 matrix, a gap weight of 16, 14, 12, 10, 8, 6 or 4, and a length weight of 1, 2, 3, 4, 5 or 6. In another embodiment, the percent identity between two nucleotide sequences is determined by using the GAP program in the GCG software package with an NWSgapdna.CMP matrix, a gap weight of 40, 50, 60, 70 or 80, and a length weight of 1, 2, 3, 4, 5 or 6.

The invention also provides variants of CRTPs that differ from the original polypeptide by one or more substitutions, deletions, and/or insertions but substantially retain the biological function of the original CRTPs. For instance, a CRTP variant can reduce, enhance or maintain the biological activity of the original CRTP. In one embodiment, the biological activity of a CRTP variant is reduced or enhanced by no more than 50%, 40%, 30%, 20%, 10% or 5% relative to the original CRTP.

Similarly, the ability of a CRTP variant to react with antigen-specific antisera can be enhanced or reduced by no more than 50%, 40%, 30%, 20%, 10% or 5% relative to the original CRTP. These variants can be prepared and evaluated by modifying the original polypeptide sequence and then determining the reactivity of the modified polypeptide with the antigen-specific antibodies or antisera.

In making such changes, the hydropathic index of amino acids can be considered. The importance of the hydropathic amino acid index in conferring interactive biologic function on a polypeptide is generally understood in the art. It is believed that the relative hydropathic character of the amino acid residue determines the secondary and tertiary structure of the resultant polypeptide, which in turn defines the interaction of the polypeptide with other molecules, such as enzymes, substrates, receptors, antibodies, antigens, and the like. It is known in the art that an amino acid may be substituted by another amino acid having a similar hydropathic index and still obtain a functionally equivalent polypeptide.

Substitution of like amino acids can also be made on the basis of hydrophilicity, particularly where the biological functional equivalent polypeptide or polypeptide fragment is intended for use in immunological embodiments. U.S. Pat. No. 4,554,101, which is incorporated hereinafter by reference, states that the greatest local average hydrophilicity of a polypeptide, as governed by the hydrophilicity of its adjacent amino acids, correlates with its immunogenicity and antigenicity, i.e., with a biological property of the polypeptide.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0) praline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). It is understood that an amino acid may be substituted for another having a similar hydrophilicity value and still obtain a biologically equivalent, such as an immunologically equivalent polypeptide. In such changes, the substitution of amino acids whose hydrophilicity values are within ±2, ±1 or ±0.5 may be used.

As outlined above, amino acid substitutions may be based on the relative similarity of the amino acid side-chain substituents, for example, their hydrophobicity, hydrophilicity, charge, size, and the like. Examples of suitable substitutions are listed in Table 4.

TABLE 4
Amino Acid Substitutions

| Original | Exemplary Residue |
|---|---|
| Residue | Substitution |
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Leu; Tyr |
| Ser | Thr |
| Thr | Ser |
| Trp | Tyr |
| Tyr | Trp; Phe |
| Val | Ile; Leu |

Polypeptide variants can be prepared by substitution, deletion and/or addition of amino acids that have minimal influence on the biological activity, immunogenicity, secondary structure, tertiary structure and/or hydropathic nature of the polypeptide. For instance, variants can be prepared by substituting, deleting or adding 1, 2, 5, or 10 amino acids residues in the original sequence. In one embodiment, polypeptide variants exhibit at least about 70%, 90%, 95% or more sequence homology to the original polypeptide.

In another embodiment, CRTPs or mutated CRTPs capable of inhibiting normal CRTP activity (dominant-negative mutants) are produced by recombinant DNA techniques. Alternative to recombinant expression, a CRTP or mutated CRTP can be synthesized chemically using standard peptide synthesis techniques.

The polynucleotide and protein sequences of the present invention can further be used as “query sequences” to perform a search against a public database to identify other family members or related sequences. Such a search can be performed using a BLAST program available at the BLAST website maintained by the National Center of Biotechnology Information (NCBI), National Library of Medicine, Washington, DC.

The invention also provides chimeric or fusion CRTPs. A fusion CRTP may contain all or a portion of a CRTP and a fusion partner (non-CRTP-related polypeptide). In one embodiment, a fusion CRTP comprises at least one biologically active portion of a CRTP. The non-CRTP-related polypeptide can be fused to the N-terminus or C-terminus of the CRTP-related polypeptide.

A peptide linker may be employed to separate the CRTP-related polypeptide from the non-CRTP-related polypeptide by a distance sufficient to ensure that each polypeptide folds into its secondary and tertiary structures. Such a peptide linker sequence is incorporated into the fusion protein using standard techniques well-known in the art. Suitable peptide linker sequences may be chosen based on the following factors: (1) their ability to adopt a flexible extended conformation; (2) their inability to adopt a secondary structure that could interact with functional epitopes on the CRTP-related polypeptide and non-CRTP-related polypeptide; and (3) the lack of hydrophobic or charged residues that might react with the polypeptide functional epitopes. Exemplary peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene, 40:39-46, 1985; Murphy et al., Proc. Natl. Acad. Sci., USA, 83:8258-8262, 1986; U.S. Pat. No. 4,935,233 and U.S. Pat. No. 4,751,180. The linker sequence may generally be from 1 to about 50 amino acids in length. Linker sequences are not required when the CRTP-related polypeptide and non-CRTP-related polypeptide have non-essential N-terminal amino acid regions that can be used to separate the functional domains and prevent steric interference.

In one embodiment, a fusion protein of the present invention is a glutathione s-transferase (GST)-CRTP fusion protein in which the CRTP sequence is fused to the C-terminus of the GST sequences. Such a fusion protein can facilitate the purification of recombinant CRTPs.

In another embodiment, the CRTP fusion proteins of the present invention are used as immunogens to produce anti-CRTP antibodies in a subject, to purify CRTP ligands and in screening assays to identify molecules which inhibit the interaction of a CRTP with a CRTP substrate.

CRTP fusion proteins used as immunogens may comprise a non-CRTP immunogenic polypeptide. In one embodiment, the immunogenic protein is capable of eliciting a recall response. In one example, a CRTP chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation.

In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence. Moreover, many expression vectors are commercially available that already encode a fusion moiety (e.g., a GST polypeptide). A CRTP-encoding polynucleotide can be cloned into such an expression vector such that the fusion moiety is linked in-frame to the CRTP.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a polynucleotide sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods.

Alternatively, the signal sequence can be linked to the protein of interest using a sequence which facilitates purification, such as with a GST domain.

The present invention also pertains to mutants of the CRTPs of the invention which function as either agonists or antagonists to the CRTPs. In one embodiment, antagonists or agonists of CRTPs are used as therapeutic agents. For example, antagonists of an up-regulated CRTPG that can decrease the activity or expression of such a gene may ameliorate cancer in a subject wherein the CRTPG is abnormally increased in level or activity. In this embodiment, treatment of such a subject may comprise administering the antagonists to a patient in need thereof so as to decrease activity or expression of the targeted CRTPG. Mutants of the CRTPs can be generated by mutagenesis, e.g., discrete point mutation or truncation of a CRTPN.

In certain embodiments, an agonist of the CRTPs retains substantially the same, or a subset, of the biological activity of the naturally occurring form of a CRTP or may enhance an activity of a CRTP. In certain embodiments, an antagonist of a CRTP can inhibit one or more of the activities of the naturally occurring form of the CRTP by, for example, competitively modulating an activity of a CRTP. Thus, specific biological effects can be elicited by treatment with a mutant CRTP of limited function. In one embodiment, treatment of a subject with a mutant CRTP having a subset of the biological activities of the naturally occurring form of the protein has fewer side effects in a subject relative to treatment with the naturally occurring form of the CRTP.

Mutants of a CRTP which function as either CRTP agonists or antagonists can be identified by screening combinatorial libraries of mutants, e.g., truncation mutants, of a CRTP for CRTP agonist or antagonist activity. A variegated library of CRTP mutants can be produced by, for example, enzymatically ligating a mixture of synthetic oligonucleotides into gene sequences such that a degenerate set of potential CRTP sequences is expressible as individual polypeptides, or alternatively, as a set of larger fusion proteins (e.g., for phage display) containing the set of CRTP sequences therein. There are a variety of methods which can be used to produce libraries of potential CRTP variants from a degenerate oligonucleotide sequence. Chemical synthesis of a degenerate gene sequence can be performed in an automatic DNA synthesizer, and the synthetic gene is then ligated into an appropriate expression vector. Use of a degenerate set of genes allows for the provision, in one mixture, of all of the sequences encoding the desired set of potential CRTP sequences. Methods for synthesizing degenerate oligonucleotides are known in the art.

In addition, libraries of fragments of a protein coding sequence corresponding to a CRTP of the invention can be used to generate a variegated population of CRTP fragments for screening and subsequent selection of mutants of a CRTP. In one embodiment, a library of coding sequence fragments can be generated by treating a double-stranded PCR fragment of a CRTP coding sequence with a nuclease under conditions wherein nicking occurs only about once per molecule, denaturing the double-stranded DNA, renaturing the DNA to form double-stranded DNA which can include sense/antisense pairs from different nicked products, removing single-stranded portions from reformed duplexes by treatment with SI nuclease, and ligating the resulting fragment library into an expression vector. By this method, an expression library, which encodes N-terminal, C-terminal and internal fragments of various sizes of the CRTPs, can be derived.

Several techniques are known in the art for screening gene products of combinatorial libraries made by point mutations or truncation, and for screening cDNA libraries for gene products having a selected property. The most widely used techniques for screening large gene libraries, which are amenable to high-throughput analysis, typically include cloning a gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify CRTP mutants (Delgrave et al. Protein Engineering, 6:327-331, 1993).

CRTPs or portions thereof may also be generated by synthetic means. For example, such polypeptides may be synthesized using any of the commercially available solid-phase techniques, such as the Merrifield solid-phase synthesis method, where amino acids are sequentially added to a growing amino acid chain. Equipment for automated synthesis of polypeptides is commercially available from suppliers such as Perkin Elmer/Applied BioSystems Division (Foster City, Calif.), and may be operated according to the manufacturer's instructions.

Antibodies

In another aspect, the invention provides antibodies that bind specifically to a CRTP of the present invention. In many embodiments, the antibodies are monoclonal or humanized antibodies.

The invention provides methods of making hybridomas which produce antibodies useful for diagnosing a patient or animal with cancer. In these methods, a CRTPP or its variant is isolated (e.g., by purification from a cell in which it is expressed or by transcription and translation of a polynucleotide encoding the protein in vivo or in vitro using known methods). A vertebrate, such as a mammal (e.g., a mouse, rabbit or sheep), is immunized using the isolated polypeptide or polypeptide fragment. The vertebrate may be immunized at least one additional time with the isolated polypeptide or polypeptide fragment, so that the vertebrate exhibits a robust immune response to the polypeptide or polypeptide fragment. Splenocytes are isolated from the immunized vertebrate and fused with an immortalized cell line to form hybridomas, using any of a variety of methods well-known in the art. Hybridomas formed in this manner are then screened using standard methods to identify one or more hybridomas which produce an antibody which specifically binds with the polypeptide or polypeptide fragment. The invention also includes hybridomas made by this method and antibodies made using such hybridomas.

An isolated CRTP, or a portion or fragment thereof, can be used as an immunogen to generate antibodies that bind the CRTPP using standard techniques for polyclonal and monoclonal antibody preparation. A full-length CRTP can be used or, alternatively, the invention provides antigenic peptide fragments of the CRTP for use as immunogens. The antigenic peptide of a CRTP comprises at least 8 amino acid residues of an amino acid sequence encoded by a CRTPG set forth in Table 1a, and encompasses an epitope of a CRTP such that an antibody raised against the peptide forms a specific immune complex with the CRTP. In many instances, the antigenic peptide comprises at least 8, 12, 16, 20 or more amino acid residues.

Immunogenic portions (epitopes) may generally be identified using well-known techniques. Such techniques include screening polypeptides for the ability to react with antigen-specific antibodies, antisera and/or T cell lines or clones. As used herein, antisera and antibodies are “antigen-specific” if they bind to an antigen with a binding affinity equal to or greater than 10⁵ M⁻¹. Such antisera and antibodies may be prepared using any method known in the art.

Exemplary epitopes are regions of a CRTP that are located on the surface of the protein, e.g., hydrophilic regions, as well as regions with high antigenicity.

Another aspect of the invention pertains to the immunologically active portions of the anti-CRTP antibodies. Examples of immunologically active portions of antibodies include F(ab) and F(ab′)2 fragments which can be generated by treating the antibody with an enzyme such as pepsin.

The anti-CRTP antibodies also include “Single-chain Fv” or “scFv” antibody fragments. The scFv fragments comprise the VH and VL domains of an antibody, wherein these domains are present in a single polypeptide chain. Generally, the Fv polypeptide further comprises a polypeptide linker between the VH and VL domains which enables the scFv to form the desired structure for antigen binding.

Additionally, recombinant anti-CRTP antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In many embodiments, humanized antibodies are desirable for therapeutic treatment of human subjects. Humanized forms of non-human (e.g., murine) antibodies are chimeric molecules of immunoglobulins, immunoglobulin chains or fragments thereof (such as Fv, Fab, Fab′, F(ab′)2 or other antigen-binding subsequences of antibodies), which contain minimal sequence derived from non-human immunoglobulin. Humanized antibodies include human immunoglobulins (recipient antibody) in which residues forming a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the constant regions being those of a human immunoglobulin consensus sequence. The humanized antibody can also comprise at least a portion of an immunoglobulin constant region (Fc), typically that of a human immunoglobulin.

Such humanized antibodies can be produced using transgenic mice which are incapable of expressing endogenous immunoglobulin heavy and light chain genes, but which can express human heavy and light chain genes. The transgenic mice are immunized in the normal fashion with a selected antigen, e.g., all or a portion of a polypeptide corresponding to a CRTP of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies.

Humanized antibodies which recognize a selected epitope can be generated using a technique referred to as “guided selection.” In this approach a selected non-human monoclonal antibody, e.g., a murine antibody, is used to guide the selection of a humanized antibody recognizing the same epitope.

In one embodiment, the antibodies to CRTP are capable of reducing or eliminating the biological function of CRTP, as is described below. That is, the addition of anti-CRTP antibodies (either polyclonal or monoclonal) to CRTP (or cells containing CRTP) may reduce or eliminate the CRTP activity. In one embodiment, at least 25%, 50%, 95, or 100% decrease in activity is achieved.

An anti-CRTP antibody can be used to isolate a CRTP of the invention by standard techniques, such as affinity chromatography or immunoprecipitation. An anti-CRTP antibody can facilitate the purification of natural CRTPs from cells and of recombinantly produced CRTPs expressed in host cells. Moreover, an anti-CRTP antibody can be used to detect a CRTP (e.g., in a cellular lysate or cell supernatant on the cell surface) in order to evaluate the abundance and pattern of expression of the CRTP. Anti-CRTP antibodies can be used diagnostically to monitor protein levels in tissue as part of a clinical testing procedure, for example, to determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or 3H.

Anti-CRTP antibodies of the invention are also useful for targeting a therapeutic agent to a cell or tissue comprising the antigen of the anti-CRTP antibody. The therapeutic agent may be a cytotoxic drug such as clicheamicin and esteramicin, an enzymatically active toxin of bacterial or plant origin, or an enzymatically active fragment (“A chain”) of such a toxin. Enzymatically active toxins and fragments thereof used are diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolacca americana proteins (PAPI, PAPII, and PAP-S), momordica charantia inhibitor, curcin, crotin, sapaonaria officinalis inhibitor, gelonin, mitogellin, restrictocin, phenomycin, and enomycin. In another embodiment, the antibodies are conjugated to small molecule anticancer drugs or cytotoxic radioactive isotopes.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfrydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional, may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, to Rodwell et al.

Where a therapeutic agent is more potent when free from the antibody portion of the immunoconjugates of the present invention, it may be desirable to use a linker group which is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler et al.).

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers that provide multiple sites for attachment can be used.

In one embodiment, antibodies to a CRTP may be used to eliminate the CRTP-containing cell population in vivo by activating the complement system, by mediating antibody-dependent cellular cytotoxicity (ADCC), or by causing uptake of the antibody coated cells by the receptor-mediated endocytosis (RE) system.

CRTP-Specific Cytotoxic Lymphocytes (T Cells)

Another aspect of the invention pertains to immunotherapeutic compositions comprising T cells specific for a CRTP. Such cells may generally be prepared in vitro or ex vivo using standard procedures. T cells may be isolated from bone marrow, peripheral blood, or a fraction of bone marrow or peripheral blood of a patient, using a commercially available cell separation system, such as the IsolexTM System, available from Nexell Therapeutics, Inc. (Irvine, Calif.). Alternatively, T cells may be derived from related or unrelated humans, non-human mammals, cell lines or cultures.

T cells may be stimulated with a CRTP or polynucleotide encoding a CRTP and/or an antigen presenting cell (APC) that expresses a CRTP. Such stimulation is performed under conditions and for a time sufficient to permit the generation of T cells that are specific for the polypeptide. In one embodiment, a CRTP or polynucleotide encoding a CRTP is present within a delivery vehicle, such as a microsphere, to facilitate the generation of specific T cells.

T cells are considered to be specific for a CRTP if the T cells specifically proliferate, secrete cytokines or kill target cells coated with the polypeptide or expressing a gene encoding the polypeptide. T cell specificity may be evaluated using any of a variety of standard techniques, such as chromium release assay or proliferation assay. In these assays, a stimulation index of more than two-fold increase in lysis and/or proliferation, compared to negative controls, indicates T cell specificity. Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with a tumor polypeptide (e.g., 100 ng/ml-100 μg/ml, or 200 ng/ml-25 μg/ml) for 3-7 days may result in at least a two-fold increase in proliferation of the T cells. Contact as described above for 2-3 hours may result in activation of the T cells, as measured using standard cytokine assays in which a two-fold increase in the level of cytokine release (e.g., TNF or IFNγ) is indicative of T cell activation. T cells that have been activated in response to a CRTP, polynucleotide encoding a CRTP, or CRTP-expressing APC may be CD4+and/or CD8+. Tumor protein-specific T cells may be expanded using standard techniques. Within many embodiments, the T cells are derived from a patient, a related donor or an unrelated donor, and are administered to the patient following stimulation and expansion.

For therapeutic purposes, CD4+ or CD8+ T cells that proliferate in response to a CRTP, polynucleotide encoding a CRTP, or APC can be expanded in number either in vitro or in vivo. Proliferation of such T cells in vitro may be accomplished in a variety of ways. For example, the T cells can be re-exposed to a CRTP, or a short peptide corresponding to an immunogenic portion of such a polypeptide, with or without the addition of T cell growth factors, such as interleukin-2, and/or stimulator cells that synthesize a CRTP. Alternatively, one or more T cells that proliferate in the presence of a CRTP can be expanded in number by cloning. Methods for cloning cells are well-known in the art, and include limiting dilution.

Vaccines

Within certain aspects, CRTP, CRTP-fusion proteins, CRTPN, CRTP-specific T cell, CRTP-presenting APC, and CRTPG-containing vectors including, but not limited to, expression vectors or gene delivery vectors, may be utilized as vaccines for cancer. Vaccines may comprise one or more such compounds/cells and an immunostimulant. An immunostimulant may be any substance that enhances or potentiates an immune response (antibody and/or cell-mediated) to an exogenous antigen. Examples of immunostimulants include adjuvants, biodegradable microspheres (e.g., polylactic galactide) and liposomes (into which the compound is incorporated). Vaccines within the scope of the present invention may also contain other compounds, which may be biologically active or inactive. For example, one or more immunogenic portions of other tumor antigens may be present, either incorporated into a fusion polypeptide or as a separate compound, within the composition of vaccine.

A vaccine may contain DNA encoding one or more CRTP or portion of CRTP, such that the polypeptide is generated in situ. As noted above, the DNA may be present within any of a variety of delivery systems known to those of ordinary skill in the art, including nucleic acid expression vectors, gene delivery vectors, and bacteria expression systems. Numerous gene delivery techniques are well-known in the art. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In one embodiment, the DNA may be introduced using a viral expression system (e.g., vaccinia or other pox virus, retrovirus, or adenovirus), which may involve the use of a non-pathogenic (defective), replication competent virus. Techniques for incorporating DNA into such expression systems are well-known to those of ordinary skill in the art. The DNA may also be “naked,” as described, for example, in Ulmer et al., (Science, 259:1745-1749, 1993). A vaccine may comprise both a polynucleotide and a polypeptide component. Such vaccines may provide for an enhanced immune response.

A vaccine may contain pharmaceutically acceptable salts of the polynucleotides and polypeptides provided herein. Such salts may be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

Any of a variety of immunostimulants may be employed in the vaccines of this invention. For example, an adjuvant may be included. Most adjuvants contain a substance designed to protect the antigen from rapid catabolism, such as aluminum hydroxide or mineral oil, and a stimulator of immune responses, such as lipid A, Bortadellci pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable micro spheres; monophosphoryl lipid A and quil A. Cytokines, such as GM-CSF or IL-2, IL-7, or IL-12, may also be used as adjuvants.

Within the vaccines provided herein, the adjuvant composition may be designed to induce an immune response predominantly of the Th1 type. High levels of Th1-type cytokines (e.g., IFNyγTNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. In one embodiment, in which a response is predominantly Th1-type, the level of Th1-type cytokines will increase to a greater extent than the level of Th2-type cytokines. The levels of these cytokines may be readily assessed using standard assays.

Any of a variety of delivery vehicles may be employed within vaccines to facilitate production of an antigen-specific immune response that targets cancer cells. Delivery vehicles include antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogenic cells.

APCs may generally be transfected with a polynucleotide encoding a CRTP (or portion or other variant thereof) such that the CRTP, or an immunogenic portion thereof, is expressed on the cell surface. Such transfection may take place ex vivo, and a composition or vaccine comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO097/24447, or the gene gun approach described by Mahvi et al., Immunology and Cell Biology, 75:456-460, 1997. Antigen loading of dendritic cells may be achieved by incubating dendritic cells or progenitor cells with the CRTPs, DNA or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

Vectors

Another aspect of the invention pertains to vectors containing a polynucleotide encoding a CRTP or a portion thereof. One type of vector is a “plasmid,” which includes a circular double-stranded DNA loop into which additional DNA segments can be ligated. Vectors include expression vectors and gene delivery vectors. The latter may be non-plasmid expression vectors, such as viral vectors.

The expression vectors of the invention comprise a polynucleotide encoding a CRTP or a portion thereof in a form suitable for expression of the polynucleotide in a host cell, which means that the expression vectors include one or more regulatory sequences, selected on the basis of the host cells to be used for expression, which are operatively linked to the polynucleotide sequence to be expressed. It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by polynucleotides as described herein (e.g., CRTPs, mutant forms of CRTPs, fusion proteins, and the like).

The expression vectors of the invention can be designed for the expression of CRTPs in prokaryotic or eukaryotic cells. For example, CRTPs can be expressed in bacterial cells such as E. coli, insect cells (using baculovirus expression vectors), yeast cells, or mammalian cells. In certain embodiments, the proteins thus prepared may be used, for example, as therapeutic proteins. Alternatively, the expression vector can be transcribed and translated in vitro, for example, by using T7 promoter regulatory sequences and T7 polymerase.

Expression of proteins in prokaryotes is often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of the recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

One strategy to maximize recombinant protein expression in E. coli is to express the protein in a host bacterium with an impaired capacity to proteolytically cleave the recombinant protein. Another strategy is to alter the polynucleotide sequence of the polynucleotide to be inserted into an expression vector so that the individual codons for each amino acid are those preferentially utilized in E. coli. Such alteration of polynucleotide sequences of the invention can be carried out by standard DNA synthesis techniques.

In another embodiment, the CRTP expression vector is a yeast expression vector. Examples of vectors for expression in yeast S. cerevisiae include pYepSec1, pMFa, pJRY88, pYES2 and picZ (Invitrogen Corp, San Diego, Calif.).

Alternatively, CRTPs of the invention can be expressed in insect cells using baculovirus expression vectors. Baculovirus vectors available for expression of proteins in cultured insect cells (e.g., Sf9 cells) include the pAc series and the pVL series.

In yet another embodiment, a polynucleotide of the invention is expressed in mammalian cells using a mammalian expression vector.

In one embodiment, the mammalian expression vector is capable of directing expression of the polynucleotide in a particular cell type (e.g., tissue-specific regulatory elements are used to express the polynucleotide). Tissue-specific regulatory elements are known in the art and may include epithelial cell-specific promoters. Other non-limiting examples of suitable tissue-specific promoters include the liver-specific albumin promoter, lymphoid-specific promoters, promoters of T cell receptors and immunoglobulins, neuron-specific promoters (e.g., the neurofilament promoter), pancreas-specific promoters, and mammary gland-specific promoters (e.g., milk whey promoter). Developmentally-regulated promoters are also encompassed, for example the α-fetoprotein promoter.

The invention provides a recombinant expression vector comprising a polynucleotide encoding a CRTP cloned into the expression vector in an antisense orientation. That is, the DNA molecule is operatively linked to a regulatory sequence in a manner which allows for expression (by transcription of the DNA molecule) of an RNA molecule which is antisense to the mRNA of the corresponding CRTPG. Regulatory sequences operatively linked to a polynucleotide cloned in the antisense orientation can be chosen to direct the continuous expression of the antisense RNA molecule in a variety of cell types. For instance viral promoters and/or enhancers, or regulatory sequences can be chosen to direct constitutive, tissue specific or cell type specific expression of antisense RNA. The antisense expression vector can be in the form of a recombinant plasmid, phagemid or attenuated virus in which antisense polynucleotides are produced under the control of a high efficiency regulatory region. The activity of the promoter/enhancer can be determined by the cell type into which the vector is introduced.

The invention further provides gene delivery vehicles for delivery of polynucleotides to cells, tissues, or a mammal for expression. For example, a polynucleotide sequence of the invention can be administered either locally or systemically in a gene delivery vehicle. Expression of the protein coding sequence can be induced using endogenous mammalian or heterologous promoters. Expression of the coding sequence in vivo can be either constituted or regulated. The invention includes gene delivery vehicles capable of expressing the contemplated polynucleotides. The gene delivery vehicle can be, without limitation, a viral vector, such as a retroviral, lentiviral, adenoviral, adeno-associated viral (AAV), herpes viral, or alphavirus vector. The viral vector can also be an astrovirus, coronavirus, orthomyxovirus, papovavirus, paramyxovirus, parvovirus, picornavirus, poxvirus, or togavirus viral vector.

Delivery of the gene therapy constructs of this invention into cells is not limited to the above mentioned viral vectors. Other delivery methods and media may be employed such as, for example, nucleic acid expression vectors, polycationic condensed DNA linked or unlinked to killed adenovirus alone, ligand linked DNA, liposome-DNA complex, eukaryotic cell delivery vehicles cells, deposition of photopolymerized hydrogel materials, handheld gene transfer particle gun, ionizing radiation, nucleic charge neutralization or fusion with cell membranes. Particle mediated gene transfer may be employed. Briefly, the sequence can be inserted into conventional vectors that contain conventional control sequences for high level expression, and then be incubated with synthetic gene transfer molecules such as polymeric DNA-binding cations like polylysine, protamine, and albumin, linked to cell targeting ligands such as asialoorosomucoid, insulin, galactose, lactose or transferrin. Naked DNA may also be employed. Uptake efficiency of the naked DNA may be improved using biodegradable latex beads.

Another aspect of the invention pertains to the expression of CRTPGs using a regulatable expression system. Systems to regulate expression of therapeutic genes have been developed and incorporated into the current viral and nonviral gene delivery vectors. These systems include Tet-on/off system, Ecdysone system, progesterone-system, and rapamycin system.

Detection Methods

As discussed earlier, CRTPGs may be used as markers for detecting or diagnosing cancers. The relative amount of a CRTPG product (polynucleotide or polypeptide) can be detected or measured by any method known in the art.

Typical methodologies for detection of a transcribed polynucleotide include, but are not limited to, Northern blot, RT-PCR, microarray, and nested-PCR. Typical methodologies for peptide detection include, but are not limited to, immunoassays (such as ELISA or Western blot), gel electrophoresis, and column chromatography.

In many embodiments, the CRTPGs themselves serve as markers for cancer. For example, an increase in genomic copies of a CRTPG, such as by duplication of the gene, may be correlated with cancer. Detection of the presence or number of copies of all or part of a CRTPG of the invention may be performed using Southern analysis or any other method known in the art.

The expression level of each CRTPG can be considered individually. Alternatively, two or more CRTPGs can be employed to increase the confidence of the analysis.

Screening Methods

The invention also provides methods or screening assays for identifying CRTPG modulators. These modulators include compounds or agents (e.g., peptides, peptidomimetics, peptoids, polynucleotides, small molecules, or other drugs) which (a) bind to a CRTP; (b) have a modulatory (e.g., up-regulatory, inductive; potentiating, stimulatory, down-regulatory, suppressive or inhibitory) effect on the activity of a CRTP; (c) have a modulatory effect on the interactions of a CRTP with one or more of its substrates; or (d) have a modulatory effect on the expression of a CRTP. An exemplary assay comprises contacting a compound of interest with cells and comparing the expression of a CRTPG in the cells before and after said contact.

In many embodiments, the compounds of interest are small molecules or biomolecules. Small molecules include, but are not limited to, inorganic molecules and small organic molecules. Biomolecules include, but are not limited to, naturally-occurring and synthetic compounds that have a bioactivity in mammals, such as polypeptides, polysaccharides, and polynucleotides. One of ordinary skill in the art will appreciate that the nature of a compound of interest may vary depending on the nature of the protein encoded by the CRTPG being investigated.

The compounds to be tested can be obtained from numerous sources, including systematic libraries of natural and/or synthetic compounds. Test compounds can also be obtained from combinatorial libraries that are constructed according to any method known in the art. Suitable libraries include biological libraries; peptoid libraries (libraries of molecules having the functionalities of peptides, but with a novel, non-peptide backbone which are resistant to enzymatic degradation but which nevertheless remain bioactive; see, e.g., Zuckermann et al., J. Med. Chem., 37:2678-85, 1994); spatially addressable parallel solid phase or solution phase libraries; libraries made by synthetic library methods requiring deconvolution; libraries made by the “one-bead one-compound” library method; and libraries made by synthetic library methods using affinity chromatography selection. See, e.g., Lam, Anticancer Drug Des., 12:145, 1997.

Screening for Inhibitors of CRTP

The invention provides methods for screening for inhibitors of CRTPs. An exemplary screening method includes obtaining samples from subjects diagnosed with or suspected of having cancer, contacting each aliquot of the samples with one of a plurality of test compounds, and comparing expression of one or more CRTPGs in each of the aliquots to determine whether any of the test compounds substantially decreases the level of expression or activity of a CRTPG relative to samples contacted with other test compounds or relative to an untreated sample or control sample. In addition, the screening methods can include directly contacting a test compound with a protein and then determining the effect of the compound on the activity of the protein.

Moreover, the invention provides methods for screening compounds capable of modulating the binding between a CRTP and a binding partner. The compounds may be either small molecules or biomolecules. The compounds can be obtained from a variety of libraries well-known in the art.

Modulators of the expression or protein activity of a CRTPG are useful as therapeutic agents for treating cancers. Such modulators (e.g., antagonists or agonists) may be formulated as pharmaceutical compositions, as described herein below. Such modulators may be used for the diagnosis, treatment, or prognosis of respective cancers.

Other methods for screening for protein inhibitors are described in U.S. Pat. Nos. 4,980,281, 5,266,464, 5,688,635, and 5,877,007, all of which are incorporated herein by reference.

High-Throughput Screening Assays

The invention provides methods of conducting high-throughput screening for compounds or agents capable of inhibiting activity or expression of a CRTP. In one embodiment, a method of high-throughput screening involves contacting the test compounds with the CRTP and then detecting the effect of the test compounds on the CRTP.

A variety of high-throughput functional assays may be used in combination to screen and/or study the reactivity of different types of activating test compounds. Since the coupling system is often difficult to predict, a number of assays may need to be configured to detect a wide range of coupling mechanisms. Numerous techniques that are capable of high-throughput and ultra high throughput screening for activities include, but are not limited to, BRET® or FRET® (both by Packard Instrument Co., Meriden, Conn.), FLIPR® (Molecular Devices Corp, Sunnyvale, Calif.), and BIACORE® systems (Biacore International AB, Uppsala, Sweden).

In one embodiment, a CRTPG encodes an orphan receptor with an unidentified ligand, and high-throughput assays are utilized to identify the ligand, and then to further identify compounds which prevent the receptor from binding to the ligand.

Detection of Genetic Alterations

The present invention provides methods for detecting genetic alterations in a CRTPG. These alterations may be associated with aberrant CRTPG expression or GRTP activity. Examples of genetic alterations include (1) deletion, addition, or substitution of one or more nucleotides; (2) a chromosomal rearrangement; (3) alteration in the non-coding regulatory sequences; (4) alteration in the mRNA level; (5) aberrant modification of the genomic sequence, such as changes in the methylation pattern; (6) abnormal RNA splicing; (7) allelic loss of a CRTPG; and (8) inappropriate post-translational modification of CRTPG products. Numerous assays are available for detecting alterations in a CRTPG. These assays include, but are not limited to, selective oligonucleotide hybridization, selective amplification, and selective primer extension, PCR (such as anchor PCR or RACE PCR), ligation chain reaction (LCR), restriction enzyme cleavage reaction, mismatch cleavage reaction, electrophoretic mobility assay, microarray, and sequencing reaction. In one embodiment, the sequence of a CRTPG of a cancer patient is determined and compared to the sequence of a CRTPG of a cancer-free patient.

Diagnostic and Prognostic Assays

The CRTPGs can be used for the diagnosis of cancer. An exemplary diagnosis method includes the steps of obtaining a biological sample from a subject of interest, determining the expression level of a CRTPG in the biological sample, and comparing the expression level to a reference expression level of the CRTPG. The biological sample can be prepared from any cell or tissue (including fluid) of interest. The reference expression level can be an average expression level of the CRTPG in normal samples. The reference expression level can also be an average expression level of the CRTPG in cancer samples.

In one embodiment, the expression levels of one or more CRTPGs in a test sample is compared to the expression levels of the same gene(s) in normal samples, and an increase in CRTPG expression in the test sample is indicative of cancer.

The expression level of a CRTPG can be determined by an immunoassay using an antibody specific for a protein encoded by the CRTPG. Suitable assays for this purpose include, but are not limited to, enzyme linked immunosorbent assays (ELISAs), Western blots, immunoprecipitation, immunofluorescence, and antibody arrays. In many embodiments, the antibody employed in the present invention has a detectable label. In many other embodiments, the antibody is indirectly labeled through a secondary antibody. The antibody can be, without limitation, a polyclonal antibody, a monoclonal antibody, a single chain antibody, or a Fab or F(ab′)₂ fragment.

The expression level of a CRTPG can also be determined by measuring the level of an RNA transcript encoded by the CRTPG. Suitable assays for this purpose include, but are not limited to, Northern blots, in situ hybridization, RT-PCR, Taqman analysis, and microarrays.

Furthermore, in vivo techniques for detection of CRTPG expression can be used. These techniques usually include introducing into a subject a labeled anti-CRTP antibody.

Prognostic assays can be devised to determine whether a subject undergoing treatment for cancer has a poor outlook for long term survival or disease progression. In one embodiment, prognosis can be determined shortly after diagnosis, for example, within a few days. By establishing CRTPG expression profiles of different stages of cancer, from onset to later stages, an expression pattern may emerge to correlate a particular expression profile to increased likelihood of a poor prognosis. The prognosis may then be used to devise a more aggressive treatment program and therefore enhance the likelihood of long-term survival and well-being.

The diagnostic assays may be used to determine the progression or severity of cancer before and after treatment. The diagnostic assays may also be used to monitor the efficacy of a therapy during clinical trials. For example, the effectiveness of a therapeutic agent can be assessed by monitoring the expression or protein activity of CRTPGs. The expression or protein activity of CRTPGs is therefore a “read-out”, which is indicative of the physiological response of the cells to the agent.

In one embodiment, a method for monitoring the effectiveness of an agent incldues the steps of (i) obtaining a pre-administration sample from a subject prior to administration of the agent; (ii) detecting the expression level or protein activity of a CRTPG in the pre-administration sample; (iii) obtaining one or more post-administration samples from the subject; (iv) detecting the expression level or protein activity of the CRTPG in the post-administration samples; (v) comparing the expression level or protein activity of the CRTPG in the pre-administration sample with that in the post-administration sample or samples; and (vi) altering the administration of the agent to the subject accordingly. According to such an embodiment, CRTPG expression or activity may be used as an indicator of the effectiveness of an agent, even in the absence of an observable phenotypic response.

The methods described herein may be performed, for example, by utilizing prepackaged diagnostic kits comprising at least one polynucleotide probe or antibody of the present invention.

In one embodiment, the comparison between CRTPG expression profiles is performed electronically, such as using a computer system. The computer system comprises a processor coupled to a memory which stores data representing the expression profiles being compared. The memory can be readable as well as rewritable. The expression data stored in the memory can be changed, retrieved or otherwise manipulated. The memory also stores one or more programs capable of causing the processor to compare the stored expression profiles. For instance, the program can execute a pattern recognition algorithm. The processor can also be coupled to a polynucleotide array scanner to receive signals from the scanner.

Methods of Treatment

The present invention provides methods for treating a cancer patient or a human who is at risk of cancer. As used herein, a treatment can be either prophylactic, therapeutic, or both. A treatment may be specifically tailored or modified based on knowledge obtained from the field of pharmacogenomics. “Pharmacogenomics,” as used herein, includes the application of genomics technologies such as gene sequencing, statistical genetics, and gene expression analysis to drugs in clinical development and on the market. The term refers to the study of how a subject's genes determine his or her response to a drug (e.g., a subject's “drug response phenotype” or “drug response genotype”). Thus, another aspect of the invention provides methods for tailoring an individual's prophylactic or therapeutic treatment with either the CRTPs or CRTP modulators (e.g., agonists or antagonists) according to that individual's drug response. Pharmacogenomics allows a clinician or physician to target prophylactic or therapeutic treatments to subjects who will most benefit from the treatment and to avoid treatment of subjects who will experience toxic drug-related side effects.

In one aspect, the invention provides a method for preventing a subject from cancer that is associated with aberrant CRTPG expression or activity. The method includes administering to the subject an anti-CRTP antibody or an agent which modulates CRTPG expression or protein activity. Administration of a prophylactic agent can occur prior to the manifestation of symptoms characteristic of the differential CRTPG protein expression, such that cancer is prevented or delayed in its progression.

Another aspect of the invention pertains to methods of modulating CRTPG protein expression or activity for therapeutic purposes. In one embodiment, the methods include contacting a cell with an agent to modulate the expression or protein activity of a CRTPG of interest. The agent can be a polynucleotide (e.g., an antisense or RNAi molecule), a polypeptide (e.g., a dominant-negative mutant of a CRTP), a naturally-occurring target molecule of a CRTP (e.g., a CRTP substrate), an anti-CRTP antibody, a CRTP modulator (e.g., agonist or antagonist), a peptidomimetic of a CRTPG protein agonist or antagonist, a small molecule drug, or a combination thereof.

The invention further provides methods for cancer treatment by targeting tumor cells with anti-CRTP antibodies that are capable of inducing antibody-dependent cellular cytotoxicity, activating the complement system and causing the uptake by receptor-medicated endocytosis, or sensitizing the cells to cytotoxins. In some embodiments, the anti-CRTP antibodies are further conjugated to a toxic drug moiety or a radiation source.

Pharmaceutical Compositions

The invention is further directed to pharmaceutical compositions for treating or preventing cancers. In one embodiment, a pharmaceutical composition of the present invention includes a modulator of the expression or protein activity of a CRTPG. In another embodiment, a pharmaceutical composition of the present invention includes an antibody specific for a protein encoded by a CRTPG, or an antisense or RNAi sequence of the CRTPG. In yet another method, a pharmaceutical composition of the present invention includes a vaccine comprising a CRTP or a fragment thereof, or an expression vector encoding the same.

The CRTPG modulators, anti-CRTP antibodies, antisense or RNAi sequences, vaccines, or other biologically active agents of the present invention can be formulated into a pharmaceutical composition. A typical pharmaceutical composition includes a pharmaceutically acceptable carrier which may include any solvent, solubilizer, filler, stabilizer, binder, absorbent, base, buffering agent, lubricant, controlled release vehicle, diluent, emulsifying agent, humectant, lubricant, dispersion media, coating, antibacterial or antifungal agent, isotonic or absorption delaying agent that is compatible with pharmaceutical administration. The use of such media and agents for pharmaceutically active substances is well-known in the art. Methods for preparing a pharmaceutical composition of an active agent are well known in the art.

A pharmaceutical composition of the invention is formulated to be compatible with its intended route of administration. Examples of routes of administration include parenteral, intravenous, intradermal, subcutaneous, oral, inhalative, transdermal topical, transmucosal, or rectal administration. Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use may include sterile aqueous solutions (where water soluble) or dispersions and sterile powders for the extemporaneous preparation of sterile injectable solutions or dispersion. For intravenous administration, suitable carriers include, but are not limited to, physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. In many embodiments, the composition is stable under the conditions of manufacture and storage and can be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is desirable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a CRTP or an anti-CRTP antibody) in the required amount in an appropriate solvent with one or a combination of ingredients enumerated above, as required, followed by filtered sterilization. In many examples, dispersions are prepared by incorporating the active compound into a sterile vehicle which contains a basic dispersion medium and the required other ingredients from those enumerated above. In the case of sterile powders for the preparation of sterile injectable solutions, example methods of preparation include vacuum drying or freeze-drying which yields a powder of the active ingredient plus any additional desired ingredient from a previously sterile-filtered solution thereof.

Oral compositions may include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds may be delivered in the form of an aerosol spray from a pressured container or dispenser which contains a suitable propellant, e.g., a gas such as carbon dioxide, or a nebulizer.

Systemic administration may be by transmucosal or transdermal means. For transmucosal or transdermal administration, penetrants appropriate to the barrier to be permeated may be used in the formulation. Such penetrants are generally known in the art, and include, for example, for transmucosal administration, detergents, bile salts, and fusidic acid derivatives. Transmucosal administration may be accomplished through the use of nasal sprays or suppositories. For transdermal administration, a bioactive agent may be formulated into ointments, salves, gels, or creams as generally known in the art.

A bioactive agent may also be prepared in the form of suppositories (e.g., with conventional suppository bases such as cocoa butter and other glycerides) or retention enemas for rectal delivery.

In one embodiment, the therapeutic moieties, which may contain a bioactive compound, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from e.g. Alza Corporation and Nova Pharmaceuticals, Inc. Liposomal suspensions (including liposomes targeted to infected cells with monoclonal antibodies to viral antigens) can also be used as pharmaceutically acceptable carriers.

In one embodiment, oral or parenteral compositions are formulated in dosage unit form for ease of administration and uniformity of dosage. Dosage unit form as used herein includes physically discrete units suited as unitary dosages for the subject to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. In many embodiments, compounds which exhibit large therapeutic indices are selected. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. In many instances, the dosage of such compounds lies within a range of circulating concentrations that includes the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The CRTPGs of the invention can be inserted into gene delivery vectors and used as gene therapy vectors. Gene therapy vectors can be delivered to a subject by, for example, intravenous administration, intraportal administration, intrabiliary administration, intra-arterial administration, direct injection into the liver parenchyma, by intramusclular injection, by inhalation, by perfusion, or by stereotactic injection. The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Kits

The invention also encompasses kits for detecting the presence of a CRTPG product in a biological sample. In one embodiment, a kit of the present invention includes an antibody or a fragment thereof, which can specifically bind to a protein encoded by a CRTPG selected from Table 1a. In another embodiment, a kit of the present invention comprises a polynucleotide probe which can hybridize under stringent conditions to an RNA transcript, or the complement thereof, of a CRTPG selected from Table 1a. The antibodies or polynucleotide probes in a kit of the present invention can be stably attached to one or more substrates to form a protein or nucleic acid array.

The kits of the present invention may also contain control or standard reagents. In many embodiments, a kit of the present invention includes an instruction for using the kit to detect CRTPG protein or polynucleotide.

The invention further provides kits for assessing the suitability of each of a plurality of compounds for inhibiting cancer in a subject. Such kits include a plurality of compounds to be tested, and a reagent (e.g., an antibody specific to a CRTP, or a probe or primer specific to a CRTPN) for assessing the expression of a CRTPG selected from Table 1a.

It should be understood that the above-described embodiments and the following examples are given by way of illustration, not limitation. Various changes and modifications within the scope of the present invention will become apparent to those skilled in the art from the present description.

### EXAMPLES

### Example 1

### Microarray and Statistical Analysis of Gene Expression Data

A combination of research tools, including oligonucleotide microarrays available from Affymetrix Inc. (Santa Clara, Calif.) and databases of biological information available from Gene Logic Inc. (Gaithersburg, Md.), were used for the statistical analysis of gene expression data.

Affymetrix oligonucleotide microarrays (commercially labeled GeneChips®) are widely used to measure the abundance of mRNA molecules in biological samples. Microarrays provide a method for the simultaneous monitoring of the expression levels of many genes in parallel. Because the oligonucleotide probes for each gene are selected and synthesized at specific locations on the array, the hybridization patterns and intensities provide direct indications of the gene identity and relative amount without the need for additional experimentation.

Matrices and algorithms are used to generate meaningful information from the intensity data obtained from microarray hybridizations. In an absolute analysis, an experimenter can determine whether a transcript is “present,” “marginal” or “absent.” Alternatively, an experimenter can obtain the average difference value which is a quantitative measure of the relative change in abundance for each transcript between a baseline and an experimental sample. The average difference is directly related to the level of expression of the transcript. In many cases, the average difference is an average of the differences between each perfect match probe cell and its control mismatch probe cell in a probe set. A probe set is a set of probes that are designed to detect one transcript. While the present call/absent call analysis is derived from the numbers of positive and negative probe pairs, the average difference metric uses probe cell intensities directly. More information on microarray analysis can be found in the Affymetrix GeneChip® Expression Analysis Manual, the entire content of which is incorporated herein by reference.

The GeneChip® Human Genome U95 Set contains five arrays that represent more than 60,000 full-length genes and EST clusters. The first array in the set is the U95Av2 Array (HG-U95Av2) which contains probes for about 12,000 full-length human gene sequences. Arrays B, C, D and E (HG-U95B, HG-U95C, HG-U95D and HG-U95E) contain probes interrogating 50,000 clusters. Each probe set in the GeneChip® Human Genome U95 Set is denoted by a “qualifier” which ends with “₁₃ at”.

BioExpress™ from Gene Logic Inc. (Gaithersburg, Md.) was employed together with the GeneChip® Human Genome U95 Set. BioExpress™ is a database containing gene expression information in a broad range of normal and diseased human tissue samples.

The NCBI RefSeqs was used to obtain mRNA and protein sequences. Transmembrane-domain prediction was made using the TMHMM2 program provided by the Center of Biological Sequence Analysis, Technical University of Denmark. Affymetrix U95 chip qualifiers for genes that encode transmembrane proteins (TM-proteins) were determined by BLASTN of the mRNA sequences of these genes against the parent sequences of the probes on the U95 chips. A unique “geneset” of qualifiers for all TM-proteins was obtained. The TM-protein gene set includes 5867 transmembrane proteins.

A statistical analysis used sixteen sample sets, including eight malignant sample sets (i.e., breast, colon, esophagus, kidney, liver, lung, prostate, and stomach cancer sample sets, respectively) and their corresponding normal counterparts. The analysis included steps of contrast analysis, fold-change analysis, and e-northern analysis using the Gene Logic GX2000 analysis suite.

The contrast analysis step included eight individual contrast analyses. In each individual contrast analysis, a pattern was defined in which one of the 8 malignant samples had higher expression values than the eight normal samples. Genes were ranked according to their t-scores in a decreasing order. The P value of the contrast analysis was calculated. Only genes with a P value smaller than 0.01 were kept. Eight files were output from the contrast analysis.

The fold change analysis step included eight fold change analyses. Each of the eight malignant samples was compared to its normal counterpart. Only genes whose expression values were more than two folds higher in malignant tissues than in normal tissues were kept. Eight files were output from the fold change analysis.

For each malignant sample set, there was one contrast analysis output and one fold change analysis output. The Affymetrix qualifiers in the two files were compared and only the common ones were kept. The common Affymetrix qualifiers were pooled for all the eight malignant sample sets and were further analyzed by the e-northern tool in GX2000.

### Example 2

### E-Northern and Global Analysis

In the e-northern analysis step, the scatter box plot of each gene was displayed for the sixteen samples. Each of the scatter box plot was studied, and only genes whose median expression value satisfy the following criteria were kept. The gene should have more than 2-fold increased expression in at least one malignant sample as compared to its normal counterpart, and the expression in the malignant sample was greater than all of the eight normal samples.

A typical e-northern figure (e.g., FIG. 1A) is composed of three parts: left, middle, and right. The left part lists the sample sets used to obtain the plot. It includes the sample set name and number of samples (in parenthesis) in the sample set. The middle part uses bars to describe the “present” percentage of individual samples in the sample set. The present percentage is proportional to the filled length of the bar, which is also labeled by the number below the bar. The right part is the major component of the figure and is composed of plots. Each plot corresponds to one sample set. The vertical bar is positioned along the x-axis according to the expression value of each individual sample. The vertical bars are color coded as follows: red means absent, blue means present and yellow means marginal. There are five numbers below the color-coded bars. The middle one is the median of expression values, the number to the left and next to the median is the 25% quantile and the number to the right and next to the median is the 75% quantile. The leftmost number represents the position that is 1.5-fold interquantile distance away from the 25% quantile. The rightmost number represents the position that is 1.5-fold interquantile distance away from the 75% quantile.

In a typical global analysis plot (e.g., FIG. 1B), the x-axis describes the sample number and the y-axis describes the expression value. Each light black dot represents the expression value of a single sample. The mean expression value for an individual tissue type was represented by a horizontal colored bar. The position of each colored bar indicates the mean expression value. The existence of blue dots below the mean bars means that the expression of those samples is called “present” in the microarray analysis.

### Example 3

### Transmembrane Hidden Markov Model (TMHMM) Analysis

The TMHMM profiles of the polypeptides encoded by the CRTPGs were generated using the TMHMM algorithm described by Krogh et al., J Mol. Biol., 305:567-580 (2001).

The first part of a TMHMM profile gives statistics and locations of the predicted transmembrane helices and intervening loop regions. If the whole sequence is labeled as inside or outside, the prediction is that it contains no membrane helices. The prediction gives the most probable location and orientation of the transmembrane helice(s) in the sequence. An algorithm called N-best (or 1-best in this case) that sums over all paths through the model with the same location and direction of the helices.

The statistics include:
Length: the length of the protein sequence;
Number of predicted TMHs: The number of predicted transmembrane helices;
Exp number of AAs in TMHs: The expected number of amino acids in transmembrane helices. If this number is larger than 18, it is likely to be a transmembrane protein (or have a signal peptide);
Exp number, first 60 AAs: The expected number of amino acids in transmembrane helices in the first 60 amino acids of the protein. If this number is more than a few, it could be warned that a predicted transmembrane helix in the N-term could be a signal peptide;
Total prob of N-in: The total probability that the N-term is on the cytoplasmic side of the membrane; and
Possible N-term signal sequence: a warning that is produced when “Exp number, first 60 AAs” is larger than 10.

The second part of a TMHMM profile is a plot of probabilities. The plot shows the posterior probabilities of inside/outside/TM helix. At the top of the plot (between 1 and 1.2) the N-best prediction is shown. The plot is obtained by calculating the total probability that a residue sits in helix, inside, or outside summed over all possible paths through the model. Sometimes the plot and the prediction seem to be contradictory. This is because the plot shows probabilities for each residue, whereas the prediction is the over-all most probable structure. Therefore the plot may be seen as a complementary source of information.

The foregoing description of the present invention provides illustration and description, but is not intended to be exhaustive or to limit the invention to the precise one disclosed. Modifications and variations are possible consistent with the above teachings or may be acquired from practice of the invention. Thus, it is noted that the scope of the invention is defined by the claims and their equivalents.

1. A pharmaceutical composition comprising a pharmaceutically acceptable carrier and a pharmaceutically effective amount of a component selected from the group consisting of:
an agent capable of modulating an expression level or protein activity of a gene selected from the group consisting of ABCC4, C20orf103, CACNA1D, CDH6, CST, ENPP3, FLJ11856, GPR54, HAVCR1, SLC6A3, SLC30A4, TRG@, and TRPM4;
an antibody specific for a polypeptide encoded by said gene; and
a T cell activated by said polypeptide.

2. The pharmaceutical composition of claim 1, wherein said component is the agent which includes a polynucleotide comprising or encoding a sequence that is capable of inhibiting or decreasing the expression of said gene by RNA interference or an antisense mechanism.

3. The pharmaceutical composition of claim 2, wherein said polynucleotide comprises or encodes an siRNA sequence selected from Table 3.

4. The pharmaceutical composition of claim 1, wherein said component is the antibody which is conjugated with a toxic moiety.

5. The pharmaceutical composition of claim 1, wherein said component is the agent which is capable of inhibiting or decreasing a protein activity of said gene.

6. A method comprising administering a therapeutically or prophylactically effective amount of the pharmaceutical composition of claim 1 to a mammal in need thereof.

7. The method of claim 6, wherein said mammal is a human who has at least one cancer selected from the group consisting of colon cancer, lung cancer, breast cancer, prostate cancer, liver cancer, kidney cancer, stomach cancer, and esophageal cancer.

8. The method of claim 7, wherein said gene is selected from the CRTPGs in Table 1b that correspond to said one cancer.

9. A vaccine formulation comprising:
a polypeptide including an immunogenic fragment encoded by a gene selected from the group consisting of ABCC4, C20orf103, CACNA1D, CDH6, CST, ENPP3, FLJ11856, GPR54, HAVCR1, SLC6A3, SLC30A4, TRG@, and TRPM4; or
an expression vector encoding said polypeptide.

10. A method comprising administering an effective amount of the vaccine formulation of claim 9 to a mammal to elicit an immune response against cancer cells expressing said gene.

11. The method of claim 10, wherein said mammal is a human who has at least one cancer selected from the group consisting of colon cancer, lung cancer, breast cancer, prostate cancer, liver cancer, kidney cancer, stomach cancer, and esophageal cancer.

12. The method of claim 11, wherein said gene is selected from the CRTPGs in Table 1b that correspond to said one cancer.

13. A method for detecting or identifying modulators of the expression or protein activity of a gene, comprising:
contacting an agent of interest with cells expressing said gene; and
comparing the expression or protein activity of said gene before and after said contacting to determine if said agent is a modulator of said gene,
wherein said gene is selected from the group consisting of ABCC4, C20orf103, CACNA1D, CDH6, CST, ENPP3, FLJ11856, GPR54, HAVCR1, SLC6A3, SLC30A4, TRG@, and TRPM4.

14. The method of claim 13, wherein said cells are infected by at least one cancer which is selected from the group consisting of colon cancer, lung cancer, breast cancer, prostate cancer, liver cancer, kidney cancer, stomach cancer, and esophageal cancer, and said gene is selected from the CRTPGs in Table 1b that correspond to said one cancer.

15. A method comprising:
detecting an expression profile of at least one gene in a biological sample of a mammal of interest, and
comparing said expression profile to a reference expression profile of said at least one gene, wherein said at least one gene is selected from the group consisting of ABCC4, C20orf103, CACNA1D, CDH6, CST, ENPP3, FLJ11856, GPR54, HAVCR1, SLC6A3, SLC30A4, TRG@, and TRPM4.

16. The method of claim 15, wherein said mammal of interest is a human who has at least one cancer selected from the group consisting of colon cancer, lung cancer, breast cancer, prostate cancer, liver cancer, kidney cancer, stomach cancer, and esophageal cancer, and said biological sample is a cancer tissue sample.

17. The method of claim 16, wherein said gene is selected from the CRTPGs in Table 1b that correspond to said one cancer.

18. The method of claim 17, wherein said reference expression profile is an average expression profile of said at least one gene in biological samples of cancer-free humans.

19. The method of claim 18, wherein said expression profile and said reference expression profile are determined by RT-PCR, nucleic acid arrays, or immunoassays.

20. A cancer diagnostic kit or device, comprising at least one of:
a polynucleotide probe capable of hybridizing under stringent conditions to a gene selected from the group consisting of ABCC4, C20orf103, CACNA1D, CDH6, CST, ENPP3, FLJ11856, GPR54, HAVCR1, SLC6A3, SLC30A4, TRG@, and TRPM4; and
an antibody specific for a polypeptide encoded by said gene.
